(19) United States
(12) Patent Application Publication
(10) Pub. No.: US 2024/0201201 A1
(43) Pub. Date: Jun. 20, 2024

(54) Biomarker Database Generation and Use

(71) Applicant: VIOME LIFE SCIENCES, INC., BELLEVUE, WA (US)

(72) Inventors: John Blume, Bellingham, WA (US); Ryan Benz, Huntington Beach, CA (US); Jeffrey Jones, Glendale, CA (US); William Smith, San Marcos, CA (US); Phong Cun, Poway, CA (US); Bruce Wilcox, Harrisonburg, VA (US)

(21) Appl. No.: 18/415,763

(22) Filed: Jan. 18, 2024

Related U.S. Application Data
(62) Division of application No. 16/089,019, filed on Sep. 27, 2018.
(60) Provisional application No. 62/315,862, filed on Mar. 31, 2016.
(60) Provisional application No. 62/444,221, filed on Jan. 9, 2017.
(60) Provisional application No. 62/454,597, filed on Feb. 3, 2017.
(60) Provisional application No. 62/475,548, filed on Mar. 23, 2017.

(51) Int. Cl. G01N 33/68; B01L 3/00; G01N 1/28; G01N 33/49; G16H 10/40; G16H 10/60; G16H 30/20; G16H 50/20; G16H 50/30; G16H 70/60

(52) U.S. Cl. CPC G01N 33/6848; B01L 3/5023; G01N 1/28; G01N 33/49; G16H 10/60; G16H 30/20; G16H 50/20; G16H 50/30; G16H 70/60; B01L 2300/0681; B01L 2300/0887; G16H 10/40

(57) ABSTRACT

Databases methods and reagents are disclosed for the generation of large amounts of biomarker data from readily obtained sample such as dried plasma spots, and for uses of such databases in the development of patient categorization or detection of changes in a patient's health status over time.

### CROSS-REFERENCE

This application claims the benefit of U.S. Prov. App. Ser. No. 62/315,862, filed Mar. 31, 2016, which is hereby explicitly incorporated herein by reference in its entirety; this application claims the benefit of U.S. Prov. App. Ser. No. 62/444,221, filed Jan. 9, 2017, which is hereby explicitly incorporated herein by reference in its entirety; this application claims the benefit of U.S. Prov. App. Ser. No. 62/454,597, filed Feb. 3, 2017, which is hereby explicitly incorporated herein by reference in its entirety; and this application claims the benefit of U.S. Prov. App. Ser. No. 62/475,548, filed Mar. 23, 2017, PCT application PCT/US2017/025580, filed Oct. 5, 2017 and U.S. patent application Ser. No. 16/089,019, filed Mar. 31, 2017 which is hereby explicitly incorporated herein by reference in its entirety.

### SUMMARY OF THE INVENTION

Provided herein are embodiments related to biomarker database generation and use in patient health classification.

Disclosed herein are methods of assessing the health status of an individual, the methods variously comprising obtaining a first measurement of a set of at least 10 fluid-borne markers from the individual at a first time point; obtaining a second measurement of the set of at least 10 markers at a second time point; and comparing the first measurement of the set of at least 10 markers to the second measurement of the set of at least 10 markers; and identifying said health status in said individual as changed when said comparing indicates a significant change between said first measurement and said second measurement. Various aspects incorporate at least one of the following elements. Some aspects comprise performing at least one immune assay on a fluid sample from the individual to obtain the first element, such as a blood sample or a plasma sample. Alternately or in combination, some aspects comprise performing at least one mass spectrometric assay on a fluid sample from the individual, such as a blood sample or a plasma sample. Mass spectrometric assays often comprise volatilizing the sample such as a dried fluid sample (for example blood or plasma). The second measurement is optionally also taken from a sample obtained from the individual. The second measurement is in some cases obtained from a set of reference circulating markers, such as a set of at least 10 markers comprising markers selected to provide information related to a particular disorder, or to a plurality of disorders. Some sets of at least 10 markers do not comprise markers selected to provide information related to a particular disorder. The size of the set varies in various embodiments, such that in some embodiments, the set of at least 10 markers comprises at least 20, 30, 40, 50, 75, 100, 200, 500, 1,000, or more than 1,000 markers. A number of comparison approaches are consistent with the disclosure herein. In some cases, a significant change between said first measurement and said second measurement comprises a change of at least 10% in a marker implicated in a disorder, or at least 5%, 15%, 20%, 25%, or greater than 25%. Alternately or in combination, a significant change between said first measurement and said second measurement comprises a change of at least 10% in a plurality of markers implicated in a common disorder, or at least 5%, 15%, 20%, 25%, or greater than 25%. In some embodiments, a significant change between said first measurement and said second measurement comprises a change of at least 20% in a marker implicated in a disorder, or at least 5%, 10%, 15%, 25%, or greater than 25%. In some aspects, a significant change between said first measurement and said second measurement comprises a change of at least 20% in a plurality of markers implicated in a common disorder, or at least 5%, 10%, 15%, 25%, or greater than 25%. In some embodiments, a significant change between said first measurement and said second measurement comprises a change of at least 10% in at least 5 markers or in at least 10, 20, 50, or more than 50 markers implicated in a common disorder. A significant change between said first measurement and said second measurement comprises a determination that said first measurement and said second measurement are statistically distinguishable in some cases. In some embodiments, the individual undergoes a treatment prior to the second time point.

Described herein are methods of constructing a biomarker panel indicative of health status of a condition in an individual, optionally comprising receiving biomarker information from a plurality of individuals, said biomarker information obtained from blood samples stored as spots on a plurality of dry solid matrixes, said biomarker information comprising at least 20 biomarkers per blood sample, and said plurality of individuals comprising individuals exhibiting the condition and individuals not exhibiting the condition; quantifying said biomarker information comprising at least 20 biomarkers per blood sample stored on a plurality of dry solid matrixes; identifying biomarkers having biomarker levels that correlate to health status of the condition; and assembling at least some of said biomarkers having biomarker levels that correlate to health status of the condition into a biomarker panel indicative of health status of a condition in an individual. The size of the biomarker information varies in terms of number of biomarkers. In some aspects, said biomarker information comprises at least 20 biomarkers, or at least 1, 2, 5, 50, 100, 200, 500, 1,000, 2,000, 5,000, 10,000, or more than 10,000 biomarkers.

Described herein are health status databases comprising biomarker accumulation levels for at least 20 biomarkers, said biomarker accumulation levels measured at a plurality of time points, such that a change in biomarker accumulation levels over time is detectable in the database. The specification is consistent with databases containing different numbers of biomarkers. In some aspects, said database comprises at least 20 biomarkers, or at least 1, 2, 5, 50, 100, 200, 500, 1,000, 2,000, 5,000, 10,000, or more than 10,000 biomarkers. The number of time points varies in many embodiments, with said plurality of time points comprises at least 3, 5, 10, 100 or more time points. Time points can be obtained over different periods of times; in some aspects, said plurality of time points comprises time points obtained over at least 1 week, 6 months, or more than 6 months. In some embodiments, said plurality of time points comprises a time point subsequent to a treatment administered to an individual. In various aspects, said database comprises biomarker accumulation levels from a single individual, or a plurality of individuals. In some embodiments, said database comprises biomarker accumulation levels obtained from a sample fluid. In some aspects, the fluid sample is whole blood, blood serum, plasma, urine, saliva, sweat, tears, cerebrospinal fluid, or any other biological fluid. Alternately or in combination, said biomarker accumulation levels are obtained from a sample fluid deposited on a dry solid matrix. In some cases, the sample is a patient tissue such as buccal cells (cheek swab), skin cells, a biopsy from an organ, or any other type of tissue containing biomarkers.

Described herein are methods of identifying a health status change in an individual comprising obtaining a dried fluid sample from the individual; measuring at least 15 circulating markers present in the dried fluid sample; comparing levels of the at least 15 circulating markers present in the dried fluid sample to levels of the at least 15 circulating markers stored in a database; and identifying a health status change in the individual when at least some of the at least 15 circulating markers present in the dried fluid sample differ significantly from levels of the at least 15 circulating markers stored in a database. Some aspects comprise identifying biomarkers present in the dried fluid sample that differ significantly from levels of the at least 15 circulating markers stored in the database, identifying a health condition related to at least some of the biomarkers present in the dried fluid sample that differ significantly from levels of the at least 15 circulating markers stored in the database; and identifying a health status change in the health condition in the individual. The dried fluid sample is whole blood, blood serum, plasma, urine, saliva, sweat, tears, cerebrospinal fluid, or any other biological fluid in some embodiments. In various cases, the health status change comprises different changes in disease states, such as colorectal health status, coronary artery status, inflammation response status, cancer status, or the status of any other disorders, conditions or other categorizations. In various aspects, the number of circulating markers for comparison to is at least 15 markers, or at least 5, 10, 20, 30, 50, 100, 200, 500, 1,000, 2,000, 5,000, or more than 5,000 markers. Alternately or in combination, the circulating markers stored in the database comprise markers obtained from at least one previous time point, optionally at least 2, 3, 5, 10, 50, 100, or more than 100 time points. In some embodiments, the circulating markers stored in the database comprise markers obtained from the individual over at least 6 months, or at least 1, 2, 5, 10, 20, 50, 100, or more than 100 months. In some cases, the circulating markers stored in a database comprise reference biomarkers that differ significantly from levels of the at least 1,000 circulating markers stored in the database, or at least 100, 200, 500, 1,000, 2,000, 5,000, 10,000, 20,000, 50,000, 100,000 or more than 100,000 circulating markers stored in the database. Alternately or in combination, the sample is collected subsequent to administering a treatment to the individual.

Described herein are methods of obtaining at least 20 features of biomarker information from a dried fluid sample comprising: obtaining a dried fluid spot on a solid matrix; subjecting the dried fluid spot to a digestion reaction to free dried fluid spot biomarkers from the solid matrix; resuspending the dried fluid spot biomarkers; subjecting the dried fluid spot biomarkers to mass spectrometric analysis comprising an LC gradient of no more than 15 minutes; and recovering at least 20 features from the mass spectrometric analysis. In various cases, the fluid sample is obtained from a variety of biological sources, such as whole blood, blood serum, plasma, urine, saliva, sweat, tears, cerebrospinal fluid, or any other biological fluid in some embodiments. In some embodiments, the solid matrix comprises backing, such as a paper backing. Alternately or in combination, digestion reactions comprise the use of enzymes (such as ArgC, AspN, chymotrypsin, GluC, LysC, LysN, trypsin, snake venom diesterase, pectinase, papain, alcanase, neutrase, snailase, cellulase, amylase, chitinase or combinations thereof) or chemical reagents (such as hydrochloric acid, formic acid, acetic acid, hydroxide bases, cyanogen bromide, 2-nitro-5-thiocyanobenzoate, hydroxylamine, or appropriate combinations thereof). In some cases, digestions are run in a solvent such as trifluoroethanol (TFE). The LC gradient involves no more than 15 minutes in some aspects, or no more than 5, 7, 10, 30, or 50 minutes. Various aspects comprise obtaining at least 20 features from the mass spectrometric analysis, or at least 2, 5, 10, 20, 30, 50, 100, 200, 500, 1,000, 2,000, 5,000, 10,000, 20,000, 50,000, 100,000, or more than 10,000 features. In some cases, the features of biomarker information are obtained such that there is a variability of no greater than 50% across duplicate spots on a solid matrix, or no greater than 3%, 5%, 7%, 10%, 20%, 25%, 50%, or 75%. Subjecting dried blood spot biomarkers to mass spectrometric analysis comprises introducing biomarker standards for at least some of the at least 20 features, in some embodiments. In various aspects, biomarker standards are introduced for at least some of the at least 5, 10, 20, 50, 100, 200, 500, 1,000 or more than 1,000 features.

Described herein are methods for using computer readable media to perform a health related assessment comprising: a) acquiring a sample from a user, wherein the sample comprises a dried biological fluid; b) receiving biometric data from said user, wherein the biometric data is provided by a tangible storage media comprising non-transitory instructions executable by a processor to cause operations to be performed; c) processing the sample and biometric data using a computer system configured for operating computer readable media, wherein processing the sample comprises extracting one or more features from the sample or biometric data and classifying the sample or biometric data using a trained classifier; and d) displaying the results of the classifier to said user on a user operated device comprising a screen through one or more graphical user interface(s). In various cases, the fluid sample is a dried blood spot, or obtained from a variety of biological sources, such as whole blood, blood serum, plasma, urine, saliva, sweat, tears, cerebrospinal fluid, or any other biological fluid in some embodiments. In various cases, the sample is acquired at a location separate from the point of collection for the sample. The tangible storage media is physically coupled to the body of the user in some cases. In some embodiments, the biometric data is transmitted by a tangible storage media housed in the user operated device. Alternately or in combination, the user operated device provides a recommendation customized to the user, based on the results of the trained classifier. Optionally, the biometric data pertains to a physical state experienced by a user, wherein the physical state is detected by a user operated device physically coupled to the user in some embodiments. In other cases, said one or more features are extracted from the sample as well as from the biometric data.

Described herein are systems for providing point-of-care detection of a health state in an individual, comprising: a) two or more user provided health data inputs, wherein at least one input comprises a biological sample, and at least one input comprises electronically provided feedback; b) a computer system configured for operating computer readable media; wherein processing the sample comprises extracting one or more features from the sample or biometric data, and classifying the sample or biometric data using a trained classifier; c) a user operated device configured to display the results of health data or biometric readings classified by the computer executable code; and d) an interface for displaying the data to the user. In some embodiments, the user operated device comprises the tangible storage media. The user operated device is coupled to the user in some aspects. In some cases, the system inputs comprise health information, such as at least one of the following: insulin levels, alertness, health, congestion, body condition, gastrointestinal health, exercise frequency, amount of sleep, diet, hunger level, time of collection, date of collection, weather at time of collection, pollen levels at time of collection, and infectious disease frequency at time of collection. Alternately or in combination, the outputs of the system comprise an assessment of health status, such as at least one of the following: expected athletic performance, expected mental capacity, oncoming illness, expected tolerance for stress, and expected response to environmental conditions. In some embodiments, the outputs of the system comprise a prediction of a health event. In other embodiments, the individual is asymptomatic for the health event. The system further comprises a tangible storage media comprising non-transitory instructions executable by a processor to cause operations to be performed including providing one or more electronically collected biometric readings in some aspects.

Described herein are methods for estimating a future health state of a user, using a non-transient computer readable media containing program instructions causing a computer to perform a method comprising the steps of: a) constructing a predictive model based on molecular, activity, or personal data received from a user, wherein the data is derived from biological samples, biometric readings, and user provided personal information; b) receiving user provided input or dataset; c) extracting multiple features from a data set, wherein the data set comprises data derived from a biological sample, biometric readings provided by a remote electronic device, and survey feedback provided by a user; d) processing input for one or more features to build a predictive model; e) providing tailored feedback to the user based on the resulting predictive model; f) revising the predictive model based on the accuracy of the feedback provided to the user; g) repeating steps (b) and (f) so as to iteratively improve the predictive accuracy of the predictive model. In some embodiments, step a) comprises a model that has been trained on data from other individuals, but not from the user. In some aspects, step (a) comprises a model that has been trained on data from the user as well as other individuals. Alternately or in combination, the tailored feedback comprises predictions of positive or negative health outcomes or precautions that the user can take. The tailored feedback is assessed with regards to key events disclosed by the user including performance related activities, in some cases.

Described herein are methods of biomarker database generation comprising identifying a biomarker set to include in a database; obtaining reference biomarker molecules that comprise biomarker components that differ in mass spectrometric migration from the protein biomarkers; obtaining at least one sample to assay for inclusion into the database; providing the reference protein biomarker molecules to the sample; subjecting the sample to mass spectrometric analysis to generate a mass spectrometric analysis output; identifying the reference biomarker molecules in the mass spectrometric analysis output; and scoring mass spectrometric spots predictably offset from the reference protein biomarker molecules as spots indicative of reference protein biomarker molecules. In various embodiments, the reference biomarker molecules comprise at least one of proteins, lipids, cholesterols, steroids, drugs, and metabolites. In some cases, the biomarker molecules comprise proteins. Alternately or in combination, the reference biomarker molecules comprise at least 10 different molecules, or at least 2, 5, 10, 20, 30, 40, 50, 100, 200, 300, 400, 500, 1,000, 2,000, 5,000, 10,000, 20,000, 50,000, 100,000 or more than 100,000 biomarker molecules. In some embodiments, the reference biomarker molecules are isotopically labeled. The reference biomarker molecules comprise molecules labeled using at least one of H2, H3, heavy nitrogen, heavy carbon, heavy oxygen, S35, P33, P32, and isotopic selenium, in some aspects. Alternately or combination, the reference biomarker molecules are chemically modified. In some cases, the reference biomarker molecules are at least one of oxidized, acetylated, methylated, and phosphorylated. In some embodiments, the reference biomarker molecules are nonhuman homologs of human proteins in the biomarker set. The sample comprises a dried sample, such as blood, blood serum, plasma, urine, saliva, sweat, tears, cerebrospinal fluid, or any other dried biological fluid in some embodiments. In some aspects, at least one sample is collected on a solid backing. In some embodiments, at least one sample comprises a collected breath sample. Consistent with the specification, the at least one sample comprises one or many samples. In some aspects, the at least one sample comprises 10 samples, or 5, 10, 50, 100, 200, 500, or 1,000 samples. In some cases, the at least one sample comprises at least 20 samples, or at least 5, 10, 50, 100, 200, 500, 1,000, or more than 1,000 samples. In some embodiments, the at least one sample comprises samples collected from an individual at different time points. In some cases, the at least one sample comprises samples collected from an individual before and after a treatment. The at least one sample comprises samples collected from a plurality of individuals in some aspects. Alternately or in combination, the at least one sample comprises samples collected from individuals differing in at least one health status. Subjecting the sample to mass spectrometric analysis comprises an LC gradient run for no more than 15 minutes or no more than 1, 3, 5, 7, 10, or 20 minutes. Alternately or in combination, digestion reactions comprise the use of enzymes (such as ArgC, AspN, chymotrypsin, GluC, LysC, LysN, trypsin, snake venom diesterase, pectinase, papain, alcanase, neutrase, snailase, cellulase, amylase, chitinase or combinations thereof) or chemical reagents (such as hydrochloric acid, formic acid, acetic acid, hydroxide bases, cyanogen bromide, 2-nitro-5-thiocyanobenzoate, hydroxylamine, or appropriate combinations thereof). In some cases, digestions are run in a solvent such as trifluoroethanol (TFE). In some aspects, identifying the reference protein biomarker molecules in the mass spectrometric analysis output is computer-automated. In some embodiments, identifying the reference protein biomarker molecules in the mass spectrometric analysis output does not comprise confirmation by a user in some embodiments. In some cases, scoring mass spectrometric spots predictably offset from the reference protein biomarker molecules as spots indicative of reference protein biomarker molecules is computer-automated. Scoring mass spectrometric spots predictably offset from the reference protein biomarker molecules as spots indicative of reference protein biomarker molecules is confirmed via ms2 mass spectrometric analysis in some aspects. Alternately or in combination, scoring mass spectrometric spots predictably offset from the reference protein biomarker molecules as spots indicative of reference protein biomarker molecules does not comprise confirmation by a user.

In some cases, native biomarker spot amounts are quantified. In some embodiments, native biomarker spot signal intensity relative to reference protein biomarker molecule spot intensity is determined. In some aspects, results of the scoring are entered into a database comprising at least 100 sample results or at least 10, 20, 50, 100, 200, 500, 1,000, 2,000, 5,000, 10,000, 100,000, 1,000,000, 1,000,000,000, or more than 1,000,000,000 sample results.

Described herein are compositions comprising a dried blood extract to which a plurality of populations of mass labeled proteins are added. In some embodiments, the reference biomarker molecules are isotopically labeled. The reference biomarker molecules comprise molecules labeled using at least one of H2, H3, heavy nitrogen, heavy carbon, heavy oxygen, S35, P33, P32, and isotopic selenium, in some aspects. Alternately or combination, the reference biomarker molecules are chemically modified. In some cases, the reference biomarker molecules are at least one of oxidized, acetylated, methylated, and phosphorylated. In some embodiments, the reference biomarker molecules are nonhuman homologs of human proteins in the biomarker set. In some cases, the plurality of populations of isotope labeled proteins comprises at least 3 populations, or at least 4, 5, 10, 15, 20, 50, 100, 1,000, 2,000, 5,000, 10,000, or more than 10,000 populations. The plurality of populations of isotope labeled proteins comprises proteins indicative of a health status when detected in circulating blood in some embodiments. Alternately or in combination, digestion reactions comprise the use of enzymes (such as ArgC, AspN, chymotrypsin, GluC, LysC, LysN, trypsin, snake venom diesterase, pectinase, papain, alcanase, neutrase, snailase, cellulase, amylase, chitinase or combinations thereof) or chemical reagents (such as hydrochloric acid, formic acid, acetic acid, hydroxide bases, cyanogen bromide, 2-nitro-5-thiocyanobenzoate, hydroxylamine, or appropriate combinations thereof). In some cases, digestions are run in a solvent such as trifluoroethanol (TFE). In some embodiments, the composition is configured on a mass spectrometric output display. In some aspects, the dried blood extract is volatilized.

Described herein are computer-implemented methods of generating a diagnostic tool for health categorization of a human subject by applying machine learning to a diagnostic instrument for health categorization, wherein the diagnostic instrument comprises inputs for a set of biomarkers, the computer implemented method comprising: on a computer system having at least one processor and a memory storing at least one computer program for execution by the at least one processor, the at least one program having instructions for: receiving as input biomarker information comprising mass spectrometric data measured from a dried liquid taken from at least one individual of known health status relevant to the health categorization; analyzing the diagnostic outcomes and the biomarker information comprising mass spectrometric data measured from the dried liquid sample taken from at least one individual of known health status relevant to the health categorization, to distinguish a panel of biomarkers informative as to health categorization; determining the accuracy of the panel of biomarkers by testing the biomarker panel against an independent source of biomarker data; generating the diagnostic tool for health categorization of a human subject, wherein the diagnostic tool comprises the panel of biomarkers; and configuring a computer device accessible by a user to receive levels of the panel of biomarkers, and to provide the user a health categorization of the human subject. The dried fluid sample is whole blood, blood serum, plasma, urine, saliva, sweat, tears, cerebrospinal fluid, or any other biological fluid in some embodiments. In some aspects, the dried liquid sample is a dried blood sample. In some embodiments, the dried liquid sample is a dried plasma sample. Alternately or in combination, the set of biomarkers comprises at least 20 mass spectrometric data points. In some aspects, the at least 20 mass spectrometric data points exhibit a median CV of no greater than 50% when mass spectrometric data is regathered from a common dried blood sample on a common collection device. In some cases, the at least 20 mass spectrometric data points exhibit a median CV of no greater than 1%, 2%, 3%, 5%, 7%, 9%, 15%, 20%, 30%, 40%, 50%, or 75% when mass spectrometric data is regathered from a common dried blood sample on a common collection device. The mass spectrometric data is regathered from a common dried blood sample on a common collection device in some embodiments. In some cases, the at least 20 mass spectrometric data points exhibit a median CV of no greater than 50% when mass spectrometric data is regathered from a plurality of dried blood samples taken from distinct collection devices or no greater than 1%, 2%, 5%, 10%, 20%, 26%, 37%, 50%, or 75% when mass spectrometric data is regathered from a plurality of dried blood samples taken from distinct collection devices. In some embodiments, the set of biomarkers comprises information about the patient, including at least one of age information, gender information, sleep information, geographical information relating to a point of sample collection, temporal information relating to a time of sample collection, and behavioral information relating to human subject alertness. Alternately or in combination, the set of biomarkers comprises at least 5 biomarkers, or at least 1, 2, 3, 4, 5, 6, 7, 8, 9, 10, 15, 20, 30, 50, 100, or more than 100 biomarkers. In some cases, the machine learning comprises a technique chosen from the group consisting of ADTree, BFTree, ConjunctiveRule, DecisionStump, Filtered Classifier, J48, J48Graft, JRip, LADTree, NNge, OneR, OrdinalClassClassifier, PART, Ridor, SimpleCart, Random Forest and SVM. The independent source comprises biomarker information obtained from a plurality of individuals of known health categorization in some embodiments. In some cases, the independent source comprises biomarker information obtained from the human subject at a plurality of time points. In some aspects, the independent source comprises biomarker information obtained from the human subject prior to administering a treatment to the subject. In some embodiments, the categorization is a disease, disorder, condition, or other categorization, such as a cancer categorization. In some cases, the categorization is at least one of a colorectal, skin, lung, throat, blood, brain, breast, and prostate cancer categorization. In some aspects, the categorization is an effective age, gender, or a demographic categorization. Consistent with the specification, the dried blood sample can be stored on a variety of surfaces. In some embodiments, the dried blood sample is stored on a planar collection matrix. In some cases, the dried blood sample is stored in a porous collection volume.

Described herein are processors comprising a memory unit configured to receive biomarker information comprising mass spectrometric data generated from at least one dried sample from a human subject; a reference unit comprising a reference dataset comprising biomarker information comprising mass spectrometric data generated from at least one dried sample of at least one individual; a processor unit configured to categorize the sample relative to the reference dataset; and an output unit configured to indicate the categorization of the sample relative to the reference dataset. The dried fluid sample is whole blood, blood serum, plasma, urine, saliva, sweat, tears, cerebrospinal fluid, or any other biological fluid in some embodiments. In some embodiments, the dried sample is at least one of a blood sample, a urine sample, a sweat sample, a tear sample and a saliva sample.

The memory unit is configured to receive dried blood sample biomarker information in some aspects. In some embodiments, the memory unit is configured to receive biomarker information comprising at least 20 mass spectrometric data points obtained per dried blood sample, or at least 10, 20, 30, 50, 100, 200, 500, 1,000, 5,000, or more than 5,000 mass spectrometric data points. In some aspects, the at least 20 mass spectrometric data points exhibit a median CV of no greater than 50% when mass spectrometric data is regathered from a common dried blood sample on a common collection device. In some cases, the at least 20 mass spectrometric data points exhibit a median CV of no greater than 1%, 2%, 3%, 5%, 7%, 9%, 15%, 20%, 30%, 40%, 50%, or 75% when mass spectrometric data is regathered from a common dried blood sample on a common collection device. The mass spectrometric data is regathered from a common dried blood sample on a common collection device in some embodiments. In some cases, the at least 20 mass spectrometric data points exhibit a median CV of no greater than 50% when mass spectrometric data is regathered from a plurality of dried blood samples taken from distinct collection devices or no greater than 1%, 2%, 5%, 10%, 20%, 26%, 37%, 50%, or 75% when mass spectrometric data is regathered from a plurality of dried blood samples taken from distinct collection devices. In some cases, the memory unit is configured to receive information about the patient, such as at least one of age information, gender information, sleep information, geographical information relating to a point of sample collection, temporal information relating to a time of sample collection, and behavioral information relating to human subject alertness. In some cases, the reference unit comprising a reference dataset comprising biomarker information is obtained from at least one individual of known health status such that the reference dataset is indicative of a health status categorization. In some aspects, the reference unit comprising a reference dataset comprising biomarker information is obtained from a population of individuals characterized as to health status. In some cases, reference unit comprising a reference dataset comprising biomarker information comprises expected biomarker levels informed by a health model. In some embodiments, the processor unit configured to categorize the sample relative to the reference dataset assigns the sample a percentile value relative to the reference dataset.

Described herein are biomarker analysis displays comprising at least 20 mass spectrometric data points obtained from a single dried sample; and at least 3 mass-shifted labeled peptide spots each indicative of an expected location of a nearby mass spectrometric data point. In some aspects, the at least 3 mass-shifted labeled peptide spots each correspond to at least one known or unknown biomarker. In some embodiments, the at least 3 mass-shifted labeled peptide spots each correspond to at least one FDA recognized quantitative biomarker(s). In some cases, the at least 3 mass-shifted labeled peptide spots each correspond to a constituent of a panel informative of a health categorization, or at least 4, 5, 6, 7, 8, 9, 10, 11, 12, 13, 14, 15, 20, 30, or more than 30 mass shifted peptide spots each correspond to a constituent of a panel informative of a health categorization. In some embodiments, the health categorization is at least one of a cancer categorization, an age categorization, a demographic categorization, a fitness categorization, a communicable disease categorization, and a non-communicable disease categorization. In some aspects, the at least 20 mass spectrometric data points exhibit a median CV of no greater than 50% when mass spectrometric data is regathered from a common dried blood sample on a common collection device. In some cases, the at least 20 mass spectrometric data points exhibit a median CV of no greater than 1%, 2%, 3%, 5%, 7%, 9%, 15%, 20%, 30%, 40%, 50%, or 75% when mass spectrometric data is regathered from a common dried blood sample on a common collection device. The mass spectrometric data is regathered from a common dried blood sample on a common collection device in some embodiments. In some cases, the at least 20 mass spectrometric data points exhibit a median CV of no greater than 50% when mass spectrometric data is regathered from a plurality of dried blood samples taken from distinct collection devices or no greater than 1%, 2%, 5%, 10%, 20%, 26%, 37%, 50%, or 75% when mass spectrometric data is regathered from a plurality of dried blood samples taken from distinct collection devices. In some aspects, one or more mass spectrometric data points are obtained from breath exudate.

Described herein are biomarker analysis displays comprising at least 100 mass spectrometric data points obtained from a single dried blood sample, wherein the at least 100 mass spectrometric data points exhibit a median CV of no greater than 7% when mass spectrometric data is regathered from a common dried blood sample. In some aspects, the at least 100 mass spectrometric data points exhibit a median CV of no greater than 26% when mass spectrometric data is regathered from a plurality of dried blood samples.

Described herein are processors comprising a memory unit configured to store data indicative of health status categorization in a comparison sample, said memory unit comprising: storage capacity configured to receive reference mass spectrometry data for at least 20 mass spectrometric values derived from each of a plurality of analyzed dried samples; storage capacity to correlate said at least 20 mass spectrometric signals derived from each of a plurality of analyzed dried blood samples to non-mass spectrometry data comprising at least one of age of sample source individual, time of collection, geographical region of collection, demographic information, blood glucose levels at time of collection, sleep history at time of collection, and mental alertness at time of collection; a comparison unit configured to receive at least one individual dataset comprising mass spectrometry data for at least 50 mass spectrometric signals derived from each of a plurality of analyzed dried blood samples and non-mass spectrometry data comprising at least one of age of sample source individual, time of collection, geographical region of collection, demographic information, blood glucose levels at time of collection, sleep history at time of collection, and mental alertness at time of collection; and compare said individual dataset to said reference mass spectrometry data, such that an assessment as to whether the individual data set differs significantly from the reference dataset is made. In some aspects the reference dataset comprises data from samples derived from at least one individual having a known health status categorization when a sample is obtained. In some cases, the reference dataset and the individual dataset are derived from a common individual or a plurality of individuals. In some cases, an individual dataset differing significantly from the reference dataset indicates that an individual source of the individual dataset does not share a health categorization of the reference dataset in some embodiments. In some embodiments, an individual dataset not differing significantly from the reference dataset indicates that an individual source of the individual dataset does share a health categorization of the reference dataset. Alternately or in combination, an individual dataset is assigned a percentile value relative to the reference dataset.

Described herein are devices for dried fluid sample collection comprising a region configured to receive a sample such that the sample dries on the region, and at least three standard markers deposited on the device such that processing of the sample introduces the marker into the sample. In some embodiments, the region configured to receive the sample comprises a surface having a planar face. In some cases, the region configured to receive the sample comprises a three-dimensional volume. In some aspects, the fluid sample is a bodily fluid such as whole blood, blood serum, plasma, urine, saliva, sweat, tears, cerebrospinal fluid, or any other biological fluid. Alternately or in combination, said biomarker accumulation levels are obtained from a sample fluid deposited on a dry solid matrix. In some cases, the sample is a patient tissue such as buccal cells (cheek swab), skin cells, a biopsy from an organ, or any other type of tissue containing biomarkers. In some embodiments, the standard marker comprises a constituent that is visualizable on a mass spectrometric output. Alternately or in combination, the standard marker comprises a constituent that has a mass that differs from a sample constituent mass by a known amount. In some aspects, the standard marker comprises a constituent that has a mass that differs from a sample constituent mass by an amount that is readily visualizable on a mass spectrometric output. The standard marker comprises a constituent that has a mass that differs from a sample constituent mass by an amount comparable to a difference between an atom and a heavy isotope of that atom in some aspects. In some embodiments, the standard marker comprises a biomolecule, such as at least a polypeptide, a lipid, a small molecule metabolite or other biomolecule. In some cases, the two samples extracted from a common collection device exhibit a CV of no greater than 6.5%, or no greater than 1%, 2%, 3%, 4%, 5%, 6%, 7%, 8%, 9%, 10%, 15%, 20%, 25%, or 50%. In some embodiments, the at least three reference biomarker molecules are isotopically labeled. The at least three reference biomarker molecules comprise molecules labeled using at least one of H2, H3, heavy nitrogen, heavy carbon, heavy oxygen, S35, P33, P32, and isotopic selenium, in some aspects. Alternately or combination, the at least three reference biomarker molecules are chemically modified or chemically labeled. In some cases, the reference biomarker molecules are at least one of oxidized, acetylated, methylated, and phosphorylated. In some embodiments, the at least three standard markers are nonhuman homologs of human proteins in the biomarker set.

Described herein are methods of identifying a biomarker signal in an existing biomarker panel selected to characterize a health condition, comprising obtaining biomarker panel level information for a plurality of samples having a known health condition status, entering the biomarker panel information to a computer configured to receive the biomarker panel information applying a machine learning algorithm to the biomarker panel information to identify a subset of markers in the biomarker panel that repeatably correlate with health condition status, thereby generating a smaller panel having a comparable health characteristic status predictive power. In some embodiments, obtaining biomarker panel level information comprises collecting dried samples from at least one source having a known health condition status. In some aspects, the dried samples are a dried bodily fluid such as whole blood, blood serum, plasma, urine, saliva, sweat, tears, cerebrospinal fluid, or any other dried biological fluid. In some aspects, obtaining biomarker panel level information comprises introducing standard markers such as heavy-labeled equivalents of panel constituents into the plurality of samples. In some aspects, assessing non-panel mass spectrometric information to identify at least one additional marker to use with the subset of markers. In some cases, the biomarker panel level information comprises mass spectrometric data. The biomarker panel level information comprises biomolecular data, such as proteomic, lipid, metabolite, or nucleic acid data. In some embodiments, accompanying data for the biomarker panel level information is obtained. In some aspects, the accompanying data comprises at least one of protein level information, RNA level information, glucose level information, sleep status information, individual alertness information, age information, gender information, demographic information, time of collection information, diet information, individual height, individual weight, individual blood pressure, environmental information at collection such as temperature, pollen status, and demographic infection status, and health condition-independent health status information such as pulmonary status information. In some embodiments, the panel comprises at least 6 markers, or at least 3, 4, 5, 6, 7, 8, 9, 10, 15, 20, 30, 50, 100, or more than 100 markers. In some aspects, the panel comprises no more than 5, 6, 7, 8, 9, 10, 15, 20, 30, 40, 50, or 100 markers

Described herein are methods of biomarker data accumulation comprising obtaining a dried fluid sample from at least one subject, volatilizing the dried fluid sample, subjecting the sample to mass spectrometric analysis, and identifying at least 20 biomarkers in the mass spectrometric analysis. In some aspects, the dried samples are a dried bodily fluid such as whole blood, blood serum, plasma, urine, saliva, sweat, tears, cerebrospinal fluid, or any other dried biological fluid. In some cases, the sample is contacted to at least one reference marker prior to mass spectrometric visualization. In some embodiments, contacting the sample to at least one reference marker comprises depositing the at least one reference marker on a solid surface prior to contacting the sample to the solid surface, adding the reference marker to the sample subsequent to resolubilizing the sample, or adding the reference marker to the sample subsequent to digesting the sample for mass spectrometric analysis. The at least one reference marker comprises a panel of reference markers that facilitates automated identification of corresponding panel constituents in the sample in some embodiments. Alternately or in combination, at least one reference marker-associated biomarker and at least one biomarker not associated with a reference marker are identified. In some embodiments, at least 50 biomarkers in the mass spectrometric analysis are identified, or at least 10, 20, 30, 40, 50, 75, 100, 200, 500, 1,000, 2,000, 5,000, 10,000, or more than 10,000 biomarkers in the mass spectrometric analysis are identified. In some embodiments, the method comprises obtaining a dried fluid sample from at least 10 subjects, or at least 5, 10, 20, 30, 50, 100, 200, 500, 1,000, 2,000, or more than 2,000 subjects. In some cases, a dried fluid sample is obtained from at least one subject at at least 2 time points. In some aspects, a treatment is administered between the at least two time points. A treatment is administered before at least one of the at least two time points in some embodiments. In some cases, the method comprising obtaining a dried fluid sample from at least one subject at at least 5 time points, or at least 2, 3, 4, 5, 7, 10, 20, 50, 100, or more than 100 time points. In some embodiments, the biomarker data comprises at least one category of information selected from a list comprising protein information, nucleic acid sequence information, nucleic acid level information, glucose information, subject body temperature, subject sleep status, subject alertness, subject diet, subject age, subject gender, subject weight, subject height, subject body mass index, subject blood pressure, subject pulse rate, time of day during collection, time of year during collection, environmental conditions during collection, pollen count during collection, environmental temperature or weather during collection, contagious disease demographic status during collection, and subject respiratory status during collection.

Described herein are computer systems comprising a memory unit configured to receive data generated through the methods consistent with the specification, and a processing unit having instructions to assess said data. In some aspects, said processing unit having instructions to assess said data comprises machine learning instructions, such as feature identification instructions classifier generation instructions. In some aspects, said feature identification instructions comprise at least one of at least one of elastic net, information gain, and random forest imputing. In some embodiments, the classifier instructions comprise at least one of logistic regression, SVM, random forest, and KNN classifier instructions. Alternately or in combination, the computer system comprises an output unit configured to display assessment results. In some embodiments, said assessment results comprise AUC information related to a panel identified through said processing unit. In some cases, said processing unit having instructions to assess said data comprises a comparison algorithm. In some cases, said comparison algorithm scores said data relative to a reference panel.

Disclosed herein are methods of predicting onset of a symptom of a genetically inherited disorder comprising identifying an individual having a genetically inherited disorder; obtaining at least one dried fluid sample from the individual; and assaying biomarker levels in the dried fluid sample via mass spectrometric analysis. In some aspects, the individual does not exhibit a symptom of the genetically inherited disorder. In some embodiments, the disorder is a cancer. In some aspects, the dried fluid samples are a dried bodily fluid such as whole blood, blood serum, plasma, urine, saliva, sweat, tears, cerebrospinal fluid, or any other dried biological fluid. In some cases, assaying biomarker levels in the dried fluid sample via mass spectrometric analysis comprises performing an untargeted mass spectrometric shotgun analysis, determining levels of particular biomarkers, or contacting the sample to at least one reference marker. In some aspects, the at least one reference marker is a heavy-labeled analogue of at least one said particular biomarker, or a nonhuman homologs of human proteins in the biomarker set. In some embodiments, the at least one reference marker is added to a collection device prior to contacting the collection device to a sample. In some cases, the at least one reference marker is added to a dried sample on a collection device or added to a resolubilized sample. The at least one reference marker is added to a sample prior to enzymatic digestion, or prior to mass spectrometric visualization in some embodiments. In some cases, assaying biomarker levels in the dried fluid sample via mass spectrometric analysis comprises determining levels of particular biomarkers and comprises performing an untargeted mass spectrometric shotgun analysis. In some embodiments, assaying biomarker levels in the dried fluid sample via mass spectrometric analysis comprises subjecting mass spectrometric data to a machine learning algorithm. In some aspects, predicting onset of a symptom of a genetically inherited disorder comprises obtaining at least two dried fluid samples, or at least 3, 4, 5, 6, 7, 8, 9, 10, or more than 10 dried fluid samples from the individual at two distinct time points. In some cases, the method comprises initiating a treatment regimen when said assaying indicates a biomarker profile indicative of onset of a symptom associated with the genetically inherited disorder. Biomarker levels comprise levels of proteins, nucleic acids, lipids, cholesterols, or other biomolecules implicated in a disease mechanism for the genetically inherited disease in some embodiments. In some aspects, said biomarker levels comprise levels of metabolites implicated in a disease mechanism for the genetically inherited disease. In some embodiments, the method comprises treating the individual to alleviate a symptom of the genetically inherited disorder. Alternately or in combination, the method comprises treating the individual prior to onset of the symptom. In some cases, the method comprises continuing sample collection and analysis to assess treatment efficacy.

Described herein are methods variously comprising obtaining a dried blood spot sample; volatilizing the dried blood spot sample; subjecting the volatilized sample to mass spectrometric analysis; and displaying at least 20 mass features from the solubilized sample. In some embodiments, at least 1 mass-shifted reference marker is added to the sample and displayed, wherein the mass-shifted reference marker maps a predictable distance from a corresponding native marker in a mass spectrometric display. In some embodiments, the at least 1 mass-shifted reference marker is isotopically labeled. The at least 1 mass-shifted reference marker is labeled using at least one of H2, H3, heavy nitrogen, heavy carbon, heavy oxygen, S35, P33, P32, and isotopic selenium, in some aspects. Alternately or combination, the at least 1 mass-shifted reference marker is chemically modified. In some cases, the at least 1 mass-shifted reference marker is at least one of oxidized, acetylated, methylated, and phosphorylated. In some embodiments, the at least 1 mass-shifted reference marker is a nonhuman homolog of a human protein mass feature. In some embodiments, at least one native marker in the mass spectrometric display is digitally quantified. In some cases, the method comprises displaying at least 5 mass-shifted reference markers added to the sample, or at least 1, 2, 5, 10, 12, 15, 17, 20, 25, 50, 75, 100, 200, or more than 200 mass-shifted reference markers added to the sample. In some embodiments, the at least 1 mass-shifted reference marker is present on a collection device prior to contacting the collection device to a sample. In some aspects, the at least 1 mass-shifted reference marker is added to the dried blood spot sample. Alternately or in combination, at least 1 mass-shifted reference marker is added to a resolubilized sample prior to mass spectrometric analysis. Consistent with the specification, the mass features comprise at least one biomolecule, such as at least one protein fragment, lipid, nucleic acid, hormone, or drug in some aspects. In some cases, the method comprises storing mass spectrometric analysis data on a computer. In some embodiments, the method comprises subjecting the mass spectrometric analysis data to a machine learning algorithm. The method comprises correlating the at least one sample to an individual of known health condition status for a health condition, and performing a machine learning analysis on the at least one native marker in some aspects. In some embodiments, the at least one sample comprises at least 10 samples, and the correlating comprises correlating each sample to an individual source of that sample. Alternatively or in combination, the method comprises displaying at least 25 mass features from the solubilized sample, or at least 5, 10, 15, 20, 25, 30, 35, 40, 45, 50, 55, 60, 100, 200, 500, 1,000, 2,000, 5,000, 10,000, or more than 10,000 mass features from the solubilized sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION

### BRIEF DESCRIPTION OF THE DRAWINGS

Some understanding of the features and advantages of the present invention will be obtained by reference to the following detailed description that sets forth illustrative embodiments, in which the principles of the invention are utilized, and the accompanying drawings.

The patent or application file contains at least one drawing executed in color. Copies of this patent or patent application publication with color drawing(s) will be provided by the Office upon request and payment of the necessary fee.

FIG. 1 depicts a Noviplex DBS Plasma Card.

FIG. 2 depicts mass spectrometric output data for 48 replicates.

FIG. 3 depicts within-card (left panel) and between card (right panel) coefficients of variation (CV) values.

FIG. 4 depicts within-card (left panel) and between card (right panel) CV values.

FIG. 5 depicts between card CV values.

FIG. 6 shows that instrument response approximating endogenous plasma concentrations.

FIG. 7 is a graph depicting protein concentration rank compared with normalized instrument response.

FIG. 8 shows detected plasma Gelsolin levels using peptide AGLNSNDAFVLK (left panel) and peptide EVQGFESATFLGYFK (right panel).

FIG. 9 shows correlation between average true positive and false positive rates for correct classes and randomized classes of sex.

FIG. 10 shows race classification of true positive and false positive rates for correct classes and randomized classes of gender.

FIG. 11 shows correlation between average true positive and false positive rates for correct classes and randomized classes of CRC status.

FIG. 12 shows correlation between average true positive and false positive rates for correct classes and randomized classes of CRC status.

FIG. 13 shows correlation between sensitivity and specificity for top model and randomized classes of CAD status.

FIG. 14 shows gradients for 30 minute (left panel) and 10 minute (right panel) gradients.

FIG. 15 shows data images for 30 minute (left) and 10 minute protocols.

FIG. 16 depicts sources of biomarkers.

FIG. 17 depicts an example of raw mass spectrometric data generated from captured exudates in breath.

FIG. 18 depicts integration of a multi-source biomarker regimen.

FIG. 19A shows mass spectrometric output for a sample.

FIG. 19B shows a mass spectrometric output for a sample overlaid with positions of exogenously added heavy labeled markers.

FIG. 20 shows marker spots subjected to automated identification and putative marker spot signals quantification for a representative list of markers.

### DETAILED DESCRIPTION OF THE INVENTION

Disclosed herein are methods, systems, databases and compositions related to targeted and untargeted health status assessment. Practice of the disclosure herein allows ongoing monitoring of a patient's health status, for example through the accurate, repeatable measurement of biomarkers such as proteins in an in vitro sample, such as an in vitro sample derived from a patient. Monitoring may be directed toward a particular health status or condition, a set of conditions, or may be untargeted such that biomarkers are monitored and a change in biomarker levels or other signal from the biomarkers signals that a health condition indicated by the biomarkers or related to the biomarkers has changed or warrants further investigation or intervention.

Disclosed herein is a demonstration of the utility of mass spectrometry for the identification and quantitation of endogenous proteins and peptides in human dried plasma samples (DPS) samples. It is demonstrated that variability in molecular feature abundances at the technical, repeated sampling, and across individuals (cohort) levels is reduced to levels facilitating diagnostic uses. The utility of shotgun proteomics to characterize and map known plasma protein components, natural sequence variants and previously unknown sequences and modifications is also demonstrated. From these results, it is disclosed that that DPS can be used as a viable platform for rapid, large scale proteomics discovery studies, and eventually for more stringent clinical applications.

Dried blood spot (DBS) samples stored on filter paper have been a popular sample collection mode for years (Deglon, J.; Thomas, A.; Mangin, P.; Staub, C. Direct analysis of dried blood spots coupled with mass spectrometry: concepts and biomedical applications. Anal Bioanal Chem 2012, 402, 2485-2498; Demirev, P. A. Dried blood spots: analysis and applications. 2013, 85, 779-789; Meesters, R. J.; Hooff, G. P. State-of-the-art dried blood spot analysis: an overview of recent advances and future trends. Bioanalysis 2013, 5, 2187-2208), and have seen applications ranging from genetic screening, infectious disease testing and drug discovery profiling. Quantitation of endogenous proteins has even been demonstrated with relative accuracy using multiple reaction monitoring mass spectrometry (Chambers, A. G.; Percy, A. J.; Yang, J.; Camenzind, A. G.; Borchers, C. H. Multiplexed quantitation of endogenous proteins in dried blood spots by multiple reaction monitoring-mass spectrometry. Mol. Cell Proteomics 2013, 12, 781-791). Additionally, detecting population wide genetic variations in abundant plasma proteins has been explored (Edwards, R. L.; Griffiths, P.; Bunch, J.; Cooper, H. J. Top-down proteomics and direct surface sampling of neonatal dried blood spots: diagnosis of unknown hemoglobin variants. J. Am. Soc. Mass Spectrom. 2012, 23, 1921-1930). DBS sampling therefor represents a convenient, simple and non-invasive method for routine molecular profiling.

Samples from dried blood or plasma spots are typically generated from the application of a drop of capillary blood applied to special filter paper. In the case of traditional dried blood spot collection, the blood sample itself is left to dry on the paper medium. In some embodiments of dried plasma spot cards, a blood sample is deposited on a filter layer that separates out the non-plasma blood components. After a specified amount of time, this filter layer is removed leaving a spot of plasma which is then left to dry prior to storage. The total time required for these types of collections can be relatively short, often no more than ten or twenty minutes including drying time. This has been demonstrated to be a robust and convenient medium for sample collection, transport, and storage (Mei, J. V.; Alexander, J. R.; Adam, B. W.; Hannon, W. H. Use of filter paper for the collection and analysis of human whole blood specimens. J. Nutr. 2001, 131, 1631S-6S). Furthermore, this sampling procedure is much simpler than that required for traditional venous blood draws and can be performed in a non-clinical setting, potentially even by the same person providing the sample. Once a blood sample has dried, many biological analytes are stabilized, and the paper or card format of the collection medium makes their transport and storage much easier compared with liquid samples. Though the application of DB S to proteomics initiatives is still in an early phase, many of the advantages inherent to DBS sample collection open new possibilities for biomarker discovery, disease testing and screening, and personalized medicine applications, including longitudinal sampling of large populations.

Historically, DBS sampling has been widely used in newborn screening. The first application was introduced by Guthrie, who used a DBS-based assay to detect phenylketonuria in newborns (Guthrie, R.; Susi, a. A Simple Phenylalanine Method for Detecting Phenylketonuria in Large Populations of Newborn Infants. Pediatrics 1963, 32, 338-343), and lead to the development of an extensive nationwide screening program in the United States for a variety of newborn disorders. DBS sampling has also been used in the context of disease monitoring (Snijdewind, I. J. M.; van Kampen, J. J. A.; Fraaij, P. L. A.; van der Ende, M. E.; Osterhaus, A. D. M. E.; Gruters, R. A. Current and future applications of dried blood spots in viral disease management. Antiviral Research 2012, 93, 309-321), therapeutic drug monitoring (Edelbroek, P. M.; van der Heijden, J.; Stolk, L. M. L. Dried blood spot methods in therapeutic drug monitoring: methods, assays, and pitfalls. Ther Drug Monit 2009, 31, 327-336), and more recently, studying biomarkers in large populations (McDade, T. W.; Williams, S.; Snodgrass, J. J. What a drop can do: Dried blood spots as a minimally invasive method for integrating biomarkers into population-based research. Demography 2007, 44, 899-925) and general proteomics applications (Chambers, A. G.; Percy, A. J.; Yang, J.; Camenzind, A. G.; Borchers, C. H. Multiplexed quantitation of endogenous proteins in dried blood spots by multiple reaction monitoring-mass spectrometry. Mol. Cell Proteomics 2013, 12, 781-791; Chambers, A. G.; Percy, A. J.; Hardie, D. B.; Borchers, C. H. Comparison of proteins in whole blood and dried blood spot samples by LC/MS/MS. J. Am. Soc. Mass Spectrom. 2013, 24, 1338-1345; Anderson, L. Six decades searching for meaning in the proteome. Journal of Proteomics 2014, 107, 24-30; Razavi, M.; Anderson, N. L.; Yip, R.; Pope, M. E.; Pearson, T. W. Multiplexed longitudinal measurement of protein biomarkers in DBS using an automated SISCAPA workflow. Bioanalysis 2016, 8, 1597-1609). Combined with targeted mass spectrometry approaches for accurate quantification of protein markers, dried blood spot sampling provides new opportunities for personalized medicine and health monitoring (Razavi, M.; Anderson, N. L.; Yip, R.; Pope, M. E.; Pearson, T. W. Multiplexed longitudinal measurement of protein biomarkers in DBS using an automated SISCAPA workflow. Bioanalysis 2016, 8, 1597-1609).

The application of liquid chromatography mass spectrometry (LC-MS) to the analysis of DBS samples has been demonstrated previously (Martin, N. J.; Bunch, J.; Cooper, H. J. Dried blood spot proteomics: surface extraction of endogenous proteins coupled with automated sample preparation and mass spectrometry analysis. J. Am. Soc. Mass Spectrom. 2013, 24, 1242-1249), however without the specific intent of assessing the feasibility of the platform to be used in biomarker discovery and personal health studies, or to map the entire observable molecular feature space from proteomic samples (Adkins, J. N.; Varnum, S. M.; Auberry, K. J.; Moore, R. J.; Angell, N. H.; Smith, R. D.; Springer, D. L.; Pounds, J. G. Toward a human blood serum proteome: analysis by multidimensional separation coupled with mass spectrometry. Mol. Cell Proteomics 2002, 1, 947-955).

Proteomics has traditionally used two primary techniques for the discovery of biomarker panels. The first approach utilizes a variety of quantitation methods (e.g., label-free quantitation, iTRAQ) on a High Resolution Mass Spectrometer (HRMS) combined with identification of peptides through MS2 spectra acquisition and database searches. An alternative approach utilizes Stable Isotope Standard (SIS) peptides in every sample measured with a QQQ mass spectrometer for improved sensitivity, quantitation and precision. Through the combination, it is possible to expand the scope of proteins that can be accurately quantitated with unequivocal identification. Here is demonstrated that the combination of SIS peptides with HRMS can quantitate 100-1000's of proteins with unequivocal identification, while simultaneously detecting the other ^{˜}30,000+ unlabeled peptides also present in the data.

Biomarkers as contemplated herein encompass a broad range of data informative of patient health. Dried blood or dried plasma is an exemplary source of biomarker information, but a broad range of biomarkers and biomarker sources are compatible with the disclosure herein. In various embodiments, markers contemplated herein include at least one of patient age, gender, glucose level, blood pressure, quantified alertness levels, mental aptitude test performance, memory performance, sleep patterns, weight measurements, calorie intake, food intake constituents, vitamin or pharmaceutical intake, prescription drug use patterns, substance abuse history, exercise patterns or exercise output quantification (in terms, for example, of distance, an estimate of calories consumed, or other measure of energy consumed or exerted), and biomolecule measurement.

Additional markers employed in some embodiments include the time and place at which a sample is collected, such as at least one of time of day, time of week, date, and season in which a sample is collected. Similarly, geographic information related to the location at which the sample is collected, and/or geographical information relating to the individual from which the sample is collected, is also included in some embodiments. This, for example, a sample may be characterized so as to identify patterns that distinguish samples taken from individuals at a polar or near polar latitude in winter from samples taken at a similar season but nearer to the equator, reflective of a differing access to sunlight. Similar data such as weather, pollen levels, influenza infection rates and/or other data relevant to the time of collection of a sample may be collected and considered as markers.

A biomolecule serving as a biomarker can be measured from a sample in any number of patient tissues, for example fluids such as in at least one of a patient's blood, blood serum, urine, saliva, cerebrospinal fluid, breath exudate or any number of other tissues or fluids. In some cases, biomolecules are measured in, for example, patient urine, collected particles or fluid droplets in breath, or in saliva or blood. Preferred embodiments comprise measurement of a plurality of biomarkers from patient blood, such as protein biomarkers.

Biomarkers derived from a patient sample such as a patient fluid, for example as circulating biomarkers in patient blood, are quantified through a number of approaches consistent with the disclosure herein. When specific markers are targeted for measurement, mass spectrometric approaches or antibodies are used to detect and in some cases to quantify the level of at least one biomarker in a sample. Alternately or in combination, biomarkers such as circulating biomarkers in a blood sample or biomarkers obtained from breath aspirate are quantified, either relatively or absolutely, through mass spectrometric approaches.

A notable aspect of some approaches herein is the generation of large amounts of marker data from biomarker measurement approaches such as mass spectrometric approaches. In various embodiments, measurements are made so that levels are determined for at least 5, 6, 7, 8, 9, 10, 11, 12, 13, 14, 15, 16, 17, 18, 19, 20, 25, 30, 35, 40, 45, 50, 60, 70, 80, 90, 100, 150, 200, 500, 1000, 2000, 5000, 10,000, 20,000 or more biomarkers in a sample. Various mass spectrometric analysis of samples, such as samples comprising proteins and/or protein fragments, facilitates generation of very large databases from which biomarker levels indicative of a patient health status are derived.

Approaches herein optionally adopt a ‘semi-targeted’ mass spectrometric approach to biomarker measurement. Samples are collected as disclosed herein. Prior to mass spectrometric analysis, internal standards, for example heavy-labeled biomolecules, are added to the samples. These standards can co-migrate with or adjacent to particular proteins or polypeptides of interest. As they are labeled, they are readily and independently detected in mass spectrometric output. When they are slightly mass-altered relative to the protein or polypeptide which they are targeting for measurement, they readily identify the unlabeled target, while migrating at a position that is displaced sufficient so as to allow the identification of the native protein or polypeptide without obscuring its signal. Such markers are used in some cases to identify proteins or polypeptides of particular interest in a sample, such as proteins recognized by the FDA to circulate in human blood and to be of particular relevance in at least one health status or health condition. Furthermore, heavy-labeled biomolecules provide the means to quantify the absolute abundance of the associated unlabeled target, providing a precise measurement of the targets level. Thus, approaches herein allow the targeted analysis of particular proteins of interest in a mass spectrometrically analyzed sample. This use of labeled markers to facilitate biomarker quantification and identification in samples allows high throughput, automated biomarker measurement in large numbers of samples as is conducive to database generation.

These approaches do not preclude the concurrent analysis of untargeted mass spectrometric signals in a sample output. That is, the labels identify peaks or signals of interest, but they do not obstruct one from observing or quantifying other unlabeled peaks or signals in a sample. Consequently, in some embodiments one can perform a targeted assay of a set of proteins of interest for which labeled mass-shifted markers are available, while at the same time collect untargeted data relating to up to every detected signal or spot in the mass spectroscopy data output.

In some examples, label-free, label, or any other mass-shifted techniques are used to identify or quantify molecular markers in the sample. For example, label-free techniques include but are not limited to the Stable Isotope Standard (SIS) peptide response. Label techniques include but are not limited to chemical or enzymatic tagging of peptides or proteins. In some examples molecular markers in the sample include all the proteins associated with a particular disease. In some examples, these proteins are selected based on several performance characteristics (i.e., peak abundance, CV's, precision, etc.).

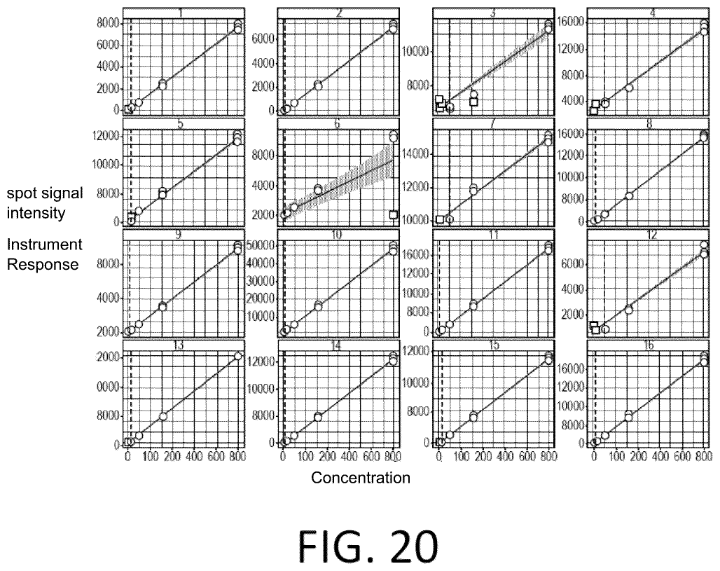

As disclosed herein, biomarkers are accurately, repeatably measured for analyses such as comparison to reference levels. Reference levels include levels of biomarkers determined from average levels of a plurality of individuals or samples for which at least one, up to a large number, of health condition statuses are known. Alternately or in combination, reference levels of biomarkers are determined from samples taken from the same individual at different times, such that temporal changes in an individual's biomarker profile are observed over time and such that a change in at least one up to a large number of biomarkers associated with a health status or condition is indicative of a change or an upcoming change in that health status or condition. A correlation is measured between concentration and spot signal strength. In one example, all polypeptide markers depicted in FIG. 20 (and representative of the larger number of polypeptide markers analyzed overall) show a clear, strong linear correlation is observed between concentration (fmol/uL, ranging from 0 to 500, as indicated on the x-axis of the bottom-most file of panels) and spot signal strength. Results (for example, those shown in FIG. 20) are used to verify that marker polypeptides are readily identified, and that their spot signal strength varies linearly with concentration, confirming both the efficacy of the identification process and their utility as markers to assist in quantification of native spots of comparable signal strength. Consistent with the specification, alternative correlations may be used in other examples to confirm efficacy and utility as markers of spots.

A single biomarker is indicative of a health status in some instances, such that a change in the biomarker level is informative as to a change in health status. Alternately or in combination, a number of biomarkers, even if individually not informative of health status or informative below a confidence level upon which information is actionable, may exhibit changes in concert such that a health condition or status for which they are commonly implicated is identified as being altered or likely to be altered in the future with a level of confidence warranting action.

In some cases, untargeted approaches are used to identify health status changes, such that biomarkers are measured or assayed for in aggregate, and changes or significant differences identified using any number of analytical techniques known to one of skill in the art are used to identify a substantial change. Such approaches are ‘untargeted’ because no particular biomarker or change is targeted for analysis. Instead, one observes the biomarkers exhibiting significant change, and then reviews the source individuals' health statuses to identify one or more health conditions or statuses for which the observed change is informative.

Alternately, biomarker data is used to identify particular markers that are informative as to a particular health status or condition, such that these markers can then be employed in stand-alone assays informative of that health status or condition. These stand-alone assays may be used in combination with untargeted marker measurements, or may be used to interpret particular subsets of untargeted biomarker measurements, or may be used in stand-alone targeted assays for a particular disease or disorder.

Analysis is facilitated by the generation of large biomarker datasets, such as datasets comprising at least 10, at least 50, at least 100, at least 1,000, at least 5,000, at least 10,000, at least 20,000 or more biomarkers from each sample analyzed. As samples are easily obtained, for example from circulating blood stored as a dried blood spot on a solid surface, databases comprising many multiples of at least 10,000 markers are readily obtained. Datasets are generated from a single individual or from multiple individuals. Multi-individual databases are in some cases partitioned by individual biomarker source, such that biomarkers obtained from individuals having a common health status can be computationally grouped, thereby facilitating identification of biomarkers indicative of the common health status and relative quantification of the native protein or polypeptide relative to the labeled reference. Datasets generated from single individuals often comprise multiple samples, such as 2, 3, 4, 5, 6, 7, 8, 9, 10, or more than 10 samples, collected at distinct time points, and may be collected at intervals of days, weeks, months or years. Some datasets comprise samples from multiple individuals and from at least one individual at multiple time points. Accordingly, health status assessments are made in various embodiments of the databases disclosed herein by at least one of comparing biomarker levels of a sample such as an in vitro analyzed sample to biomarker levels of reference samples having known biomarker levels and known health status, and comparing biomarker levels of a sample such as an in vitro analyzed sample to biomarker levels of other samples taken from the same individual at the same or different time points.

Biomarker datasets, such as biomarker datasets generated from mass spectrometry data, or other source such as protein array data or immunological assays, variously comprise biomarkers corresponding to at least one of 1) known proteins or fragments mapping to known proteins of known function and known role in at least one heath status or disorder, 2) known proteins or known fragments mapping to known proteins of known function but unknown role in a health status or disorder, 3) unknown or unidentified proteins or fragments, such as fragments identified by mass spectrometric migration, time of flight, mass-to-charge ratios or mass, elution or other position in a mass spectrometric output, but that have not been mapped to or identified with a particular protein of known function, but that nonetheless are in some cases relevant as markers for a health status or condition, for example due to their identifiable difference in levels between samples that differ in a known or hypothesized health status or health condition. When the protein or other biomarkers are known, their detection in a mass spectrometry analyzed dataset is facilitated in some cases by the introduction of labeled biomarkers, such as heavy isotope labeled biomarkers that are detectable independent of the biomarker mass spectrometry labeling approach, and that migrate in mass spectrometry analyses at a repeatable, predictable offset from a native or naturally occurring biomarker in the sample.

Accordingly, in various embodiments herein, marker data is useful in identifying a protein or set of proteins that differ between samples, such as individuals of differing health status or within a single individual at different time points, such that the identity of the biomarkers indicate a health condition or health status difference between individuals or in the individual at one time point compared to another. For example, when two samples from a single individual are observed to differ at a number of biomarkers corresponding to proteins implicated in a health condition, such as colorectal cancer or heart disease, then analysis of the dataset or datasets indicates that one of the individuals may have or be at increased risk of having the health condition, such as colorectal cancer or heart disease. A partial list of health conditions for which biomarkers are informative includes cardiovascular diseases (heart disease), hyperproliferative diseases (for example, cancer), neural diseases (for example, Alzheimer's disease), autoimmune diseases (for example, lupus), metabolic diseases (such as obesity), inflammatory diseases (for example arthritis), bone diseases (such as osteoporosis), gastrointestinal diseases (such as ulcers) blood diseases (such as sickle cell anemia), infections (for example, bacterial, viral, and fungal infections), and chronic fatigue syndrome.

Alternately, in various embodiments herein, marker data is used to identify a biomarker or set of biomarkers or other markers that are informative of a health condition, independent of whether the biomarkers are implicated in the health condition or even, in some cases, independent of whether the biomarkers are correlated to a known or characterized protein or other biomarker. That is, in a comparison of biomarker datasets taken from individuals known to differ in a health status or condition, one is enabled to identify biomarkers that individually in a combination are indicative of that health status or condition, even in cases when the identity of a protein or proteins from which the marker is derived are not known. The identity of at least one biomarker alone or in a panel is in some cases immaterial to the efficacy of the marker alone or in a panel as an indicator or predictor of health status. So long as a health status is known or hypothesized to differ between samples or groups of samples, then biomarkers that consistently differ between the samples may be informative as to the health status of de novo tested samples. The identity of the biomarker or biomarkers is in some cases later determined, but it does not in all cases need to be determined for it to be useful alone or in a panel as a predictor of the health status.

A number of biomarker sample collection methods are consistent with the disclosure herein. In some exemplary cases, samples are collected from patient blood by depositing blood onto a solid matrix such as is done by spotting blood onto a paper or other solid backing, such that the blood spot dries and its biomarker contents are preserved. The sample can be transported, such as by direct mailing or shipping, or can be or stored without refrigeration. Alternately, samples are obtained by conventional blood draws, saliva collection, urine sample collection, or by collection of exhaled breath. As mentioned above, samples are in some cases augmented through the collection of additional health data such as at least one of dietary information, sleep information, exercise data, glucose level assays, blood pressure analysis, alertness or other mental acumen test results, and other behavioral information.

Non-tissue based markers, such as age, mental alertness, sleep patterns, measurement of exercise or activity among others, and/or biomarkers that are readily measured at the point of collection, such as glucose levels, blood pressure measurements, are collected using any number of methods known in the art. In various embodiments, collection or transmission of such data comprises electronically transmitting such data, for example using a personal or hand-held device.

### Sample Collection Approaches

Fluid samples are collected using an appropriate collection device. In some cases, the Noviplex Plasma Prep Card (Novilytic Labs) is used for plasma collection. In one experiment to assess technical variability, a generic pooled plasma sample is obtained and placed on the collection device. In one example, a human plasma sample is purchased from Bioreclamation IVT and spotted on an appropriate number of Noviplex cards. In some cases, plasma is spotted on 16 individual Noviplex cards. In another experiment to assess variability due to repeated sampling, adult blood plasma is collected from a single member of a research group who volunteered the donation. In some cases, collection from a single member comprises collecting a number of samples (for example, 12 samples) over the course of a time period, for example 2 hours. Samples can be obtained from a single finger prick deposited on a collection device, for example onto a single Noviplex Card. Alternative methods of sample collection and sample collection devices may also be used. In another experiment to assess the variability across a number of different individuals (cohort sampling), adult blood plasma from each of a group of participants, for example, 99 participants (collected by ProMedDx LLC, Norton, Mass.), are spotted on an appropriate plasma collection device. In one example, plasma is spotted on Noviplex Plasma Prep Duo Cards. Alternative collection devices may also be employed. A particular cohort may comprise a mixture of individuals with different races and sexes. In one example, the cohort may comprise 64 Caucasians (32 males, 32 females) and 35 African Americans (30 males, 5 females). After sample collection, the sample collection device can be transported under appropriate shipping conditions for analysis. In one example, DPS cards are transported to Applied Proteomics, Inc. under standard ambient shipping conditions with desiccant only, for LC-MS analysis. Alternative shipping conditions may also be used in other examples. Consistent with the specification, alternative methods of sample collection may be utilized.

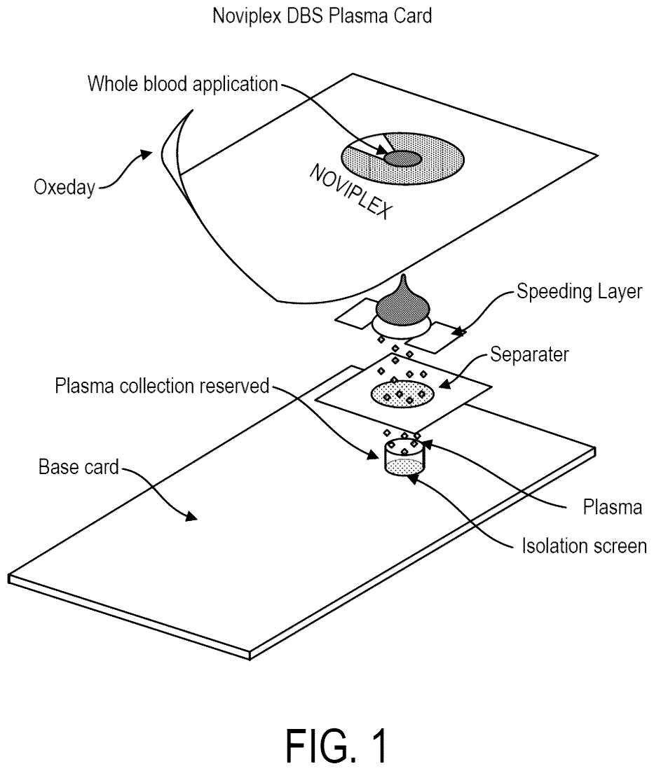

According to some collection protocols, a sample containing biomarkers of interest is applied to a collection device for analysis. In some examples, the sample is a fluid such as whole blood, blood serum, urine, saliva, sweat, tears, cerebrospinal fluid, or any other biological fluid. Alternately, a sample is a patient tissue such as buccal cells (cheek swab), skin cells, a biopsy from an organ, or any other type of cell containing biomarkers. The sample is often subsequently processed before analysis to obtain specific fractions. In one example whole blood samples are applied to a collection device, such as Noviplex DBS Plasma Card as indicated in FIG. 1A separation technique may be used to separate plasma from whole blood for analysis. For example, a separation technique may involve drawing whole blood through a separating layer comprising a separator to isolate plasma, and directing plasma to a plasma collection reservoir. The plasma then contacts an isolation screen on a case card. In some examples, the sample may be dried for storage and later analysis.

A sample containing biomarkers of interest is applied to a collection device for analysis. In some examples, the sample is a fluid such as whole blood, blood serum, urine, saliva, sweat, tears, cerebrospinal fluid, or any other biological fluid. Alternately, a sample is a patient tissue such as buccal cells (cheek swab), skin cells, a biopsy from an organ, or any other type of cell containing biomarkers. The sample is often subsequently processed before analysis to obtain specific fractions. In one example whole blood samples are applied to a collection device, such as Noviplex DBS Plasma Card as indicated in FIG. 1A separation technique may be used to separate plasma from whole blood for analysis. For example, a separation technique may involve drawing whole blood through a separating layer comprising a separator to isolate plasma, and directing plasma to a plasma collection reservoir. The plasma then contacts an isolation screen on a case card. In some examples, the sample may be dried for storage and later analysis.

The sample spot on the collection device is placed into an individual well for digestion. In some examples, the biomarker obtained from the patient contains biomolecules. Biomolecules optionally include biopolymers in some examples. Examples of biopolymers include but are not limited to proteins, lipids, polysaccharides, or nucleic acids. In some examples, biomolecules are digested prior to analysis using digestion reagents that may include enzymes or chemical reagents with or without a solvent for a suitable period of time at a suitable temperature. Enzymatic digestions in some examples include but are not limited to the use of ArgC, AspN, chymotrypsin, GluC, LysC, LysN, trypsin, snake venom diesterase, pectinase, papain, alcanase, neutrase, snailase, cellulase, amylase, chitinase or combinations thereof. In one example, trypsin in solvent TFE for an extended duration such as at least 1, 2, 3, 4, 5, 6, 7, 8, 9, 10, 12, or 24 hours is used to digest proteins. Non-enzymatic digestions include the use of acids or bases in some examples. Suitable acids include hydrochloric acid, formic acid, acetic acid, or combinations thereof. Suitable bases include hydroxide bases. Other non-enzymatic digestions include use of chemical reagents such as cyanogen bromide, 2-nitro-5-thiocyanobenzoate, hydroxylamine, or combinations thereof. Other examples of non-enzymatic digestion may include electrochemical digestion. Combinations of enzymatic and non-enzymatic digestion methods may be utilized in some examples. After the digestion is quenched, in some examples the digested sample is transferred to plate and dried down. In some collection devices, the sample is applied to a three dimensional absorbent structure rather than being spotted onto a two dimensional plane. In one example, the blood collection device is a Neoteryx Mitra blood collection device. In some examples, the blood can be dried and stored at room temperature prior to analysis. Other examples may include the use of alternative sample preparation protocols, consistent with the specification.

Plasma samples are prepared for analysis using a variety of methods. In one example, LC-MS analysis is utilized. For example, the collection layer containing plasma from a collection device may be transferred to a plate and centrifuged. In one example, a Noviplex card is transferred to a single well in a 2 mL 96-well plate, and then centrifuged for a specific time and speed, for example 2 minutes at 500 g. Alternative methods of plasma purification may also be employed. A plate containing plasma is then processed for analysis. Processing may include denaturation, reduction, alkylation, and/or digestion. For example, a plate is transferred to a Tecan EVO150 liquid handler for denaturation with 50% 2,2,2-trifluoroethanol (TFE, Arcos) in 100 mM ammonium bicarbonate (Sigma). Alternative denaturation reagents and/or solvents in different concentrations may also be employed. The sample may be reduced, for example with 200 mM DL-dithiothreitol (Sigma) or with any other appropriate reducing agent. The sample may be alkylated, for example with 200 mM iodoacetamide (Arcos), and the alkylation terminated, for example, with 200 mM DL-dithiothreitol. Other appropriate alkylation reagents may be used with or without additional termination reagents. The sample may be digested under appropriate digestion conditions, for example with trypsin (Promega) for 16 hr. at 37° C., and quenched with 5 uL of neat formic acid. Other digestion methods, for example other enzymatic digestion methods are also utilized for alternative amounts of time at alternative temperatures. Digested samples are transferred to a sampling plate, and the solvent is removed as needed for analysis. For example, samples are transferred to a 330-uL 96-well plate (Costar) for lyophilization. Samples are then reconstituted for analysis under the appropriate conditions. In one example, for technical and repeat sampling sets, samples are reconstituted with solvents, such as a mixture of water and acetonitrile with formic acid, vortexed for a period of time, and centrifuged at a speed for a time period. For example, samples may be dissolved in 50 uL of 97/3 water/acetonitrile with 0.1% formic acid, vortexed at 500 rpm for 15 minutes, and centrifuged for 2 minutes at 500 g for analysis. This same step may also be used for a cohort sample set, with an optional modification. In one example using modified conditions, 76 uL of 97/3 water/acetonitrile with 0.1% formic acid is used to account for the additional plasma collected by the Noviplex Duo card used in this example as stated by the card manufacturer. Consistent with the specification, other sample reconstitution conditions may be used in other examples.

Both technical and repeat sampling sets are processed within a single plate, such as a 96-well plate to minimize inter-day effects. The cohort sample set is processed in sample groups with subject samples and pooled plasma samples for QC/normalization purposes. In one example, 11 sample groups are used, each with 9 subject samples and two pooled plasma samples. Each sample group is then analyzed on the LC-MS platform shortly after processing, for example the day following the completion of sample processing. In one example, the total number of samples comprises 99 study samples and 18 QC/normalization samples. Consistent with the specification, alternative numbers of study and normalization samples are employed in other examples.

LC-MS data from each sample is collected on an appropriate instrument with an appropriate ionization source, for example a quadrupole time-of-flight (Q-TOF) mass spectrometer (Agilent 6550) coupled to ultra-high performance liquid chromatography (UHPLC) instrument (Agilent 1290), with an electrospray ionization (ESI) source. LC flow rates are optimized based on sample conditions and pressures. In one example, the LC flow rate is optimized at 450 uL/min and remains stable around 650 bar. LC-MS conditions are not limited to those described below; they may be optionally modified in terms of sample volume, sample concentration, or column type. In other examples, high purity nitrogen gas is used for collision induced dissociation (CID). In one example, autosamplers (for example, Agilent 1290) are used to automate the delivery of a 10 uL volume from a 96-well plate containing samples of approximately 3 ug/uL digested plasma for chromatographic separation on an C18 column (for example, Waters ACQUITY UPLC CSH) optionally with dimensions of 2.1×150 mm, 1.7 um particle size. For example, an LC mobile phase A is comprised of 0.1% formic acid in water and mobile phase B is comprised of 0.1% formic acid in acetonitrile. LC conditions are not limited to those described above, but may utilize different solvents in different ratios. For example, A 30 min UHPLC gradient is used to separate the analytes with the following linear segments: mobile phase B increased from 3% to 6% in 0.5 min, 6% to 10% in 2 min, 10% to 30% in 18.75 min, 30% to 40% in 5 min, 40% to 80% in 1.25 min, and stayed at 80% for 1.25 min before returning to 3% B in 0.75 min. The UHPLC gradient may be modified in terms of % B and time (alternative linear segments) in other examples to provide optimal separation of analytes.

For the assessment of technical variability, plasma samples from the sample collection devices (for example, each of the 16 DPS) cards are analyzed by LC-MS with multiple replicates, for example three technical replicates each, resulting in a total of 48 injections. In one example, all 48 injections successfully produce data for subsequent quantitative analysis. The assessment of repeated sampling is performed with plasma samples from the sample collection devices (for example, each of the 12 DPS cards), that subsequently are analyzed by LC-MS in multiple replicates, for example, four replicates. In one example, of 48 injections, one failed during data collection (replicate 3 from DPS card 8), resulting in a final set of 47 injections for analysis. Different sample collection devices, or conditions may produce a different final data set. Finally, for the assessment of cohort variability, a single injection from a collection device (for example each of the 99 individual DPS cards) is analyzed by LC-MS. In some examples, all of samples are suitable for quantitative analysis.

The peptide and protein content of the collection device is assessed by analysis of a number of injections from a single pooled source. For example, a collection of DPS samples is assessed by LC-MS using a total of 24 injections from a single pooled source. Data is collected in MS1/MS2 mode so that feature identifications can be made concurrently with the quantitative MS1 data. Tandem mass spectrometry data is collected via a second fragmentation method, such as collision induced dissociation (CID), in which an MS1 survey scan is followed by fragmentation of other precursor ions, such as the three most abundant precursor ions. In one example, the MS2 survey scans are acquired utilizing the method of gas-phase fractionation (Spahr, C. S.; Davis, M. T.; McGinley, M. D.; Robinson, J. H.; Bures, E. J.; Beierle, J.; Mort, J.; Courchesne, P. L.; Chen, K.; Wahl, R. C.; Yu, W.; Luethy, R.; Patterson, S. D. Towards defining the urinary proteome using liquid chromatography-tandem mass spectrometry. I. Profiling an unfractionated tryptic digest. Proteomics 2001, 1, 93-107.; Davis, M. T.; Spahr, C. S.; McGinley, M. D.; Robinson, J. H.; Bures, E. J.; Beierle, J.; Mort, J.; Yu, W.; Luethy, R.; Patterson, S. D. Towards defining the urinary proteome using liquid chromatography-tandem mass spectrometry II. Limitations of complex mixture analyses. Proteomics 2001, 1, 108-117; Scherl, A.; Shaffer, S. A.; Taylor, G. K.; Kulasekara, H. D.; Miller, S. I.; Goodlett, D. R. Genome-specific gas-phase fractionation strategy for improved shotgun proteomic profiling of proteotypic peptides. Anal. Chem. 2008, 80, 1182-1191; Peiris-Pagès, M.; Smith, D. L.; Györffy, B.; Sotgia, F.; Lisanti, M. P. Proteomic identification of prognostic tumour biomarkers, using chemotherapy-induced cancer-associated fibroblasts. Aging (Albany N.Y.) 2015, 7, 816-838), optimized within a number of distinct mass ranges, selected to evenly distribute precursor targets and optimize the density of data collection.

### Output Processing and Feature Determination

Molecular features are extracted from the MS1 data of the collection device (for example, a DPS card) injections using a feature detection algorithm such as OpenMS (Sturm, M.; Bertsch, A.; Gröpl, C.; Hildebrandt, A.; Hussong, R.; Lange, E.; Pfeifer, N.; Schulz-Trieglaff, O.; Zerck, A.; Reinert, K.; Kohlbacher, O. OpenMS—an open-source software framework for mass spectrometry. BMC Bioinformatics 2008, 9, 163). In one example, feature detection is performed in 3-dimensional space along the m/z, LC time and abundance axes to find and associate the isotopic peaks from peptide molecular features in the LC-MS data.

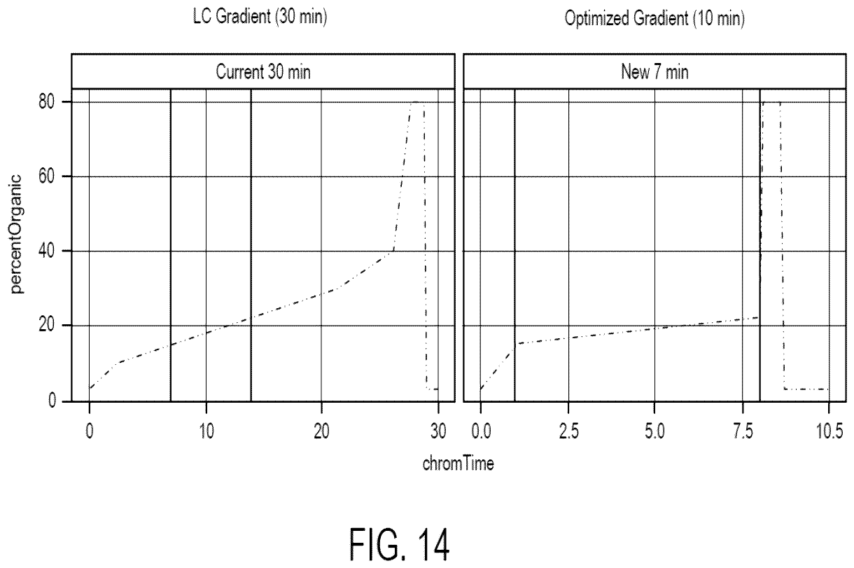

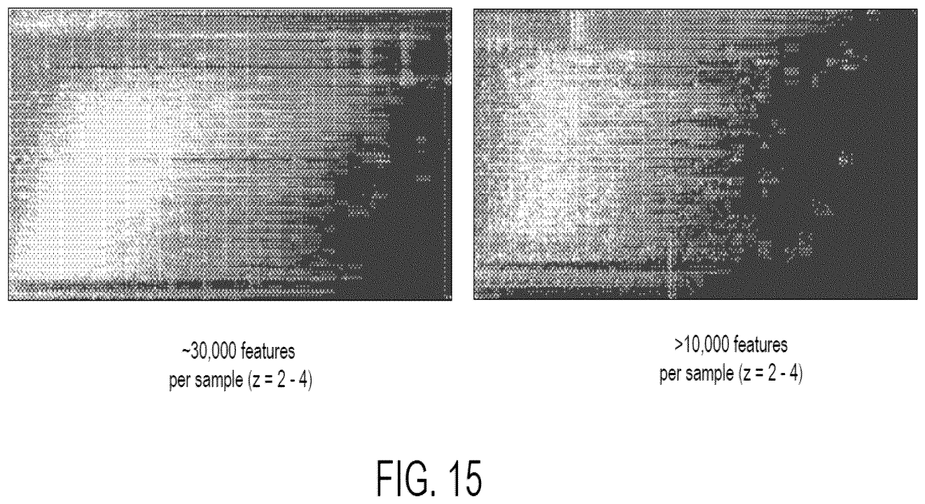

Mass spectrometric analyses are used to generate a number of features per sample by employing a liquid chromatography gradient for a period of time. For example, the number of features ranges from about 10 to more than 80,000 features, including at least, exactly or no more than 10 to 50, 50 to 100, 100 to 1000, 1000 to 2000, 2000 to 3000, 3000 to 5000, 5000 to 10,000, 10,000 to 20,000, 20,000 to 30,000, 30,000 to 40,000, 40,000 to 50,000, 50,000 to 60,000, 60,000 to 70,000, 70,000 to 80,000, 80,000 to 90,000, 90,000 to 100,000, or greater than 100,000 features. In some examples, the gradient time is 30 minutes, or less than 30 minutes. In some examples, a portion of the gradient disproportionately responsible for a high density of meaningful data is identified, and an optimized gradient is developed so as to focus on this more information dense region of the gradient. A modified protocol allows a rapid analysis of samples without sacrificing overall sample biomarker quality in some workflows consistent with the disclosure herein. In one example, by focusing on this region, the gradient time is reduced from 30 to 10 minutes (FIG. 14) and the information content of biomarkers remained greater than 10,000 (FIG. 15.) Additional examples consistent with the specification involve the optimization of other LC-MS operational variables (including but not limited to solvent, column type, column size, or fragmentation source) that result in an improved sample throughput. Consistent with the specification, analysis instrument ionization sources for feature identification include but are not limited to electrospray ionization (ESI), fast atom bombardment (FAB) or matrix-assisted laser desorption/ionization (MALDI). Consistent with the specification, mass analyzers for feature identification include but are not limited to linear ion traps, 3D ion traps, triple quadrupole ion traps, FT-cyclotrons, single or dual time-of-flight (TOF), or combinations thereof. In some examples, analysis instruments are ionization sources combined with one or more mass analyzers. In some examples, analysis instruments include but are not limited to ESI-QqQ (electrospray ionization-triple quadrupole), ESI-qTOF (electrospray ionization-quadrupole time-of-flight), or MALDI-QqTOF (MALDI-double quadrupole-TOF).

At the completion of the feature detection process, the final output consists of a list of molecular features, each comprising but not limited to grouped isotopic peaks, the monoisotopic m/z value, LC time, and the 3-dimensional integrated abundance of the feature's monoisotopic peak. In one example, the MS1 data analyzed here resulted in ˜40,000 features per injection. For some quantitative analysis, the 3-dimensional monoisotopic peak integrated areas is used to represent the quantitative abundance value for each molecular feature. Consistent with the disclosure herein, alternative molecular features may be extracted, and alternative features detected.

At the completion of each of a number of variability experiments (for example, 3 variability experiments), extracted molecular features are optionally associated across experiment injections based upon their m/z and LC time values. A simple LC alignment algorithm is employed prior to cross-sample feature association to account for sample-to-sample LC variability. Next, feature filtering is applied to retain only features appearing in a minimum percentage of the total number of injections, for example at least 25%. Molecular feature abundance CV's are then calculated on these filtered features, individually for each feature, both within and between collection devices (for example, DPS cards) for the technical and repeated sampling experiments, and across the individual cards for the cohort variability experiment. For the between-card abundance CV determination, feature values are first averaged within card to obtain per-card feature estimates, and the between-card CV values are then computed using the per-card abundance estimates.

Tandem mass spectrometry data are analyzed, for example, using a 2014 version of the Human UniProt DB, a 6-frame translation of the entire human genome (NCBI, 304.5 million unique peptide sequences), and all known human protein sequence variants (UniProt, 65,935 unique peptide sequences generated from 12511 open reading frames, ORFs). Mass matching tolerances for precursor ion and fragment ions are set, in some examples at 100 ppm and 150 ppm respectively (Haas, W.; Haas, W.; Faherty, B. K.; Gerber, S. A.; Elias, J. E.; Beausoleil, S. A.; Bakalarski, C. E.; Li, X.; Villen, J.; Gygi, S. P. Optimization and Use of Peptide Mass Measurement Accuracy in Shotgun Proteomics. Mol. Cell Proteomics 2006, 5, 1326-1337). Remaining unsequenced high quality MS2 spectra are searched again using a non-precursor dependent search for novel PTM discovery (Weng, R. R.; Chu, L. J.; Shu, H.-W.; Wu, T. H.; Chen, M. C.; Chang, Y.; Tsai, Y. S.; Wilson, M. C.; Tsay, Y.-G.; Goodlett, D. R.; Ng, W. V. Large precursor tolerance database search—A simple approach for estimation of the amount of spectra with precursor mass shifts in proteomic data. Journal of Proteomics 2013, 91, 375-384; Chick, J. M.; Kolippakkam, D.; Nusinow, D. P.; Zhai, B.; Rad, R.; Huttlin, E. L.; Gygi, S. P. A mass-tolerant database search identifies a large proportion of unassigned spectra in shotgun proteomics as modified peptides. Nat Biotechnol 2015, 33, 743-749).

Feature determination and quantification is accomplished through a number of approaches, such as the following. In one example, each of the precursor dependent database searches start with commonly found post-translational modifications (no modifications, Carbamidomethyl), followed by a round of searches whereby laboratory-induced modifications are added, (Carbamylation, Acetylation, Oxidation, Deamidation, Carboxymethylation), then biological modifications are added (Phosphorylation, Ubiquitinylation, Methylation, DiMethylation). Each search is allowed to have up to a preset number of simultaneous modifications, for example, 3 modifications. Consistent with the specification, other examples may search alternative databases for alternative post-translational modifications.

Protein reconstruction involves homology mapping all peptide sequences of significance to the Human UniProt DB using a variety of reported methods (Nesvizhskii, A. I.; Keller, A.; Kolker, E.; Aebersold, R. A statistical model for identifying proteins by tandem mass spectrometry. 2003, 75, 4646-4658; Kearney, P.; Butler, H.; Eng, K.; Hugo, P. Protein Identification and Peptide Expression Resolver: Harmonizing Protein Identification with Protein Expression Data. J. Proteome Res. 2008, 7, 234-244; Kearney, P.; Butler, H.; Eng, K.; Hugo, P. Protein Identification and Peptide Expression Resolver: Harmonizing Protein Identification with Protein Expression Data. J. Proteome Res. 2008, 7, 234-244; Mujezinovic, N.; Schneider, G.; Wildpaner, M.; Mechtler, K.; Eisenhaber, F. Reducing the haystack to find the needle: improved protein identification after fast elimination of non-interpretable peptide MS/MS spectra and noise reduction. BMC Genomics 2010, 11, S13).

### Biomarker Database Development, Biomarker Sources, and Characteristics

Some methods, databases and panels relate to health assessment, heath categorization or health status assessment relying upon marker database development.

Marker data are obtained from at least one source as disclosed herein. A focus of the disclosure herein is biomarkers obtained from fluids, such as blood, plasma, saliva, sweat, tears and urine. Particular attention is paid to blood, and to plasma extracted from a blood sample, such as prior to drying the blood sample. However, alternative biomarker sources are contemplated and are consistent with the disclosure herein.

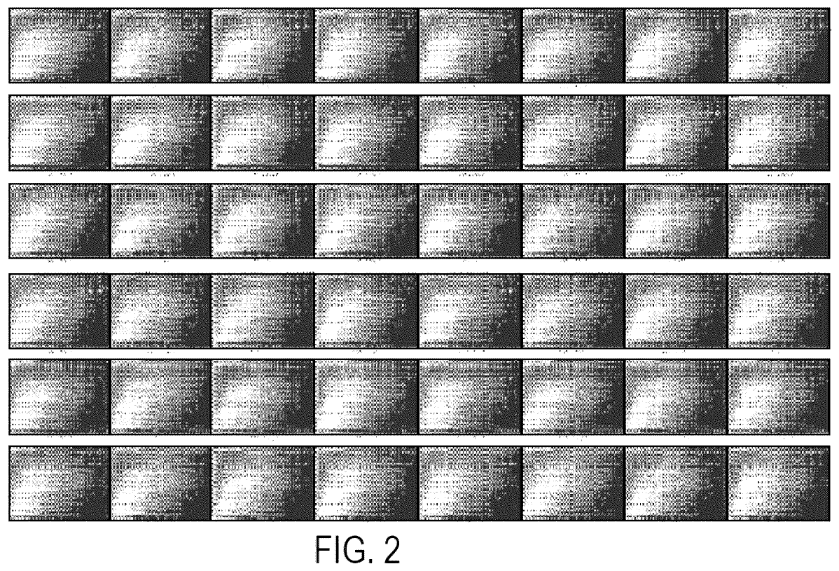

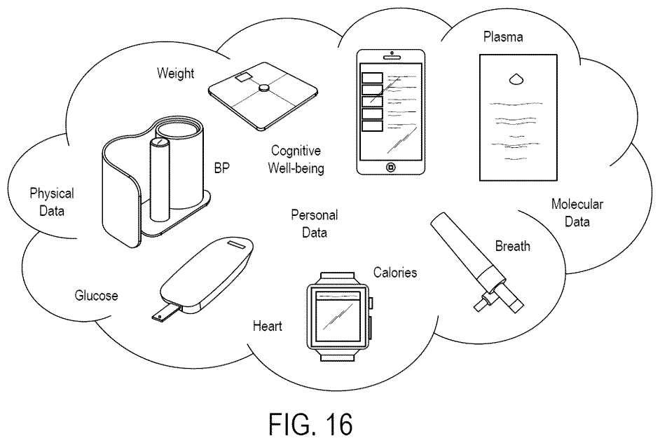

Marker sources include but in some cases are not limited to proteomic and non-proteomic sources. Examples of sources of markers include age, mental alertness, sleep patterns, measurement of exercise or activity, or biomarkers that are readily measured at the point of collection, such as glucose levels, blood pressure measurements, heart rate, cognitive well-being, alertness, weight, are collected using any number of methods known in the art. Some marker sources are indicated in, for example, FIG. 16. Exemplary biomarker sources include circulating biomarkers in a blood or plasma sample or biomarkers obtained from breath aspirate that are quantified, either relatively or absolutely, through mass spectrometric approaches or using antibodies, or other immunological or non-immunological approaches. Examples of raw data obtained from such sources is given in FIGS. 2, 15 and 17.

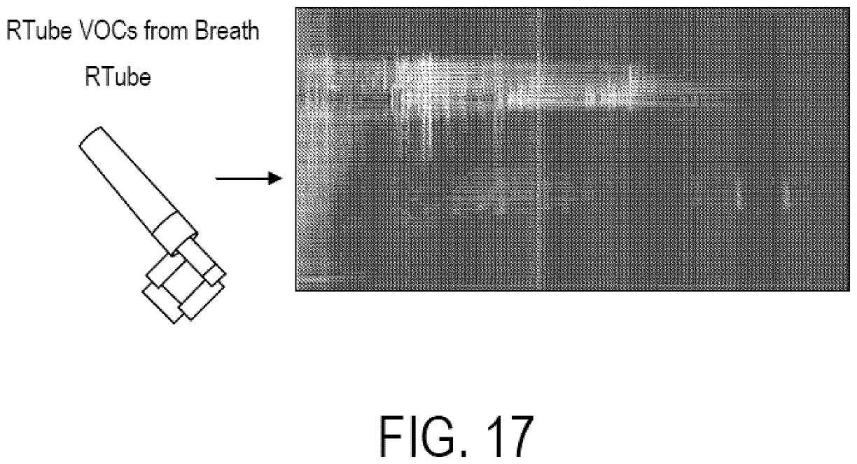

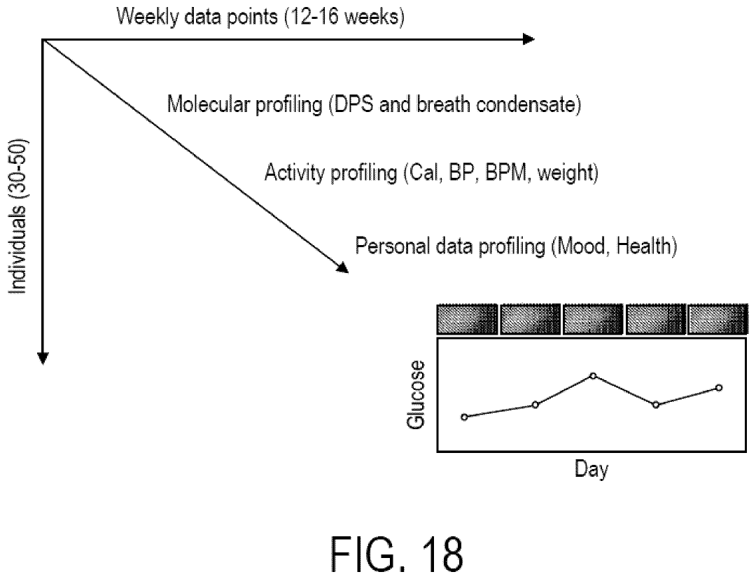

In some examples, biomarker data sources include physical data, personal data and molecular data. In some examples, physical data sources include but are not limited to blood pressure, weight, heart rate, and/or glucose levels. In some examples, personal data sources include cognitive well-being. In some examples, molecular data sources include but are not limited to specific protein markers. In some examples, molecular data includes mass spectrometric data obtained from plasma samples obtained as dried blood spots and/or obtained from captured exudates in breath samples. One example of raw mass spectrometric data generated from captured exudates in breath is given in FIG. 17. In some examples, biomarker and other marker data from multiple sources are integrated as part of a multi-source marker regimen, and depicted in FIG. 18.

Additionally, some biomarkers are informative of the environment from which a sample is taken, such biomarkers include, weather, time of day, time of year, season, temperature, pollen count or other measurement of allergen load, influenza or other communicable disease outbreak status.

Biomarker-based data in some cases comprises large amounts of potentially relevant biomarkers. In particular, databases disclosed herein comprise in some cases at least 10, at least 50, at least 100, at least 1,000, at least 5,000, at least 10,000, at least 20,000 or more obtained from a single sample, such as a readily obtained sample deposited as a blood spot on a solid surface, such as seen in FIG. 1. Collecting biomarker data from blood spots, alone or in combination with other readily available sources of biomarker or other marker data, dramatically facilitates database generation. Samples are collected in some cases remote from a health facility or laboratory, and are stored and transmitted without costly refrigeration. Nonetheless, as indicated in the description including the figures and examples herein, large quantities of biomarker data are obtained, facilitating database generation.

Databases are variously developed from a plurality of individuals or sample sources collected at a single time point or a plurality of time points, at one sample per individual or multiple samples per individual, collected at one or at multiple time points from one or multiple individuals. In some cases databases are developed from a single individual or other single sample source through repeated sampling and biomarker processing over time, so as to produce a ‘longitudinal’ or temporally progressing database. Some databases comprise both a plurality of individuals and a plurality of collection time points.

In some cases, an individual or a sample taken from an individual at a particular time is associated with a health condition or health status for that individual at that time. Thus, biomarkers or other markers obtained from a sample are associated with a health condition or health status, such as presence, absence, or a relative level of severity of a disorder.

Data is often collected and analyzed over time. Groups of markers that change over time and are linked may be monitored together, for example, markers implicated in glucose regulation such as glucose levels, mental acuity, and patient weight. In some examples, differences in these markers may be indicative of disease states or disease progression. Similarly, in some cases data is collected in combination with administration of a treatment regimen or intervention, such that data is collected both before and after a treatment such as a pharmaceutical treatment, chemotherapy, radiotherapy, antibody treatment, surgical intervention, a behavioral change, an exercise regimen, a diet change, or other health intervention. Data analysis can indicate whether a treatment regimen was successful, is impacting a biomarker profile such as reducing marker levels or slowing to health decline-related change in biomarker levels, or otherwise continues to be relevant to a patient. In some examples, a report detailing the patient's markers can inform a medical professional.

Biomarker levels that vary in concert with differences in health condition or health status are in some cases selected for validation as individual indicators or as members of panels indicative of health condition or health status. Often, individual markers are identified that correlate with health condition or status, but overall predictive value is improved when multiple markers, particularly markers that do not strictly co-vary, nonetheless are independently predictive of health status.

In some cases the biomarkers are further identified as to protein source, such that protein specific analysis is performed. The protein identifies are analyzed, for example so as to shed light on a biological mechanism underlying a correlation between a biomarker level and a health condition or status.

When the protein or other biomarkers are known, their detection in a mass spectrometry analyzed dataset is facilitated in some cases by the introduction of labeled biomarkers into a sample prior to mass spectrometric analysis. Labeled markers are markers such as heavy isotope labeled biomarkers that are detectable independent of the biomarker mass spectrometry labeling approach, and that migrate in mass spectrometry analyses at a repeatable, predictable offset from a native or naturally occurring biomarker in the sample. By identifying the labeled markers in a mass spectrometric output, and in light of the known offset of the native biomarker relative to its labeled counterpart, one can readily identify the expected position and size of a biomarker spot on a mass spectrometric output. Such labeling facilitates accurate, automated calling of large numbers of biomarkers in a mass spectrometric sample, such as 100, 200, 300, 400, 500, 600, 700, 800, 900, 1,000, or more than 1,000 biomarkers in a sample.

Biomarkers that map to known proteins are often examined as to whether their measurement using immunology-based methods yields results that are similarly informative as compared to mass spec data. In such cases, the biomarkers are in some cases developed as constituents of stand-alone panels for the detection or assessment of a specific health condition or health status, such as a cancer heath status (e.g., colorectal cancer health status), coronary artery health status, Alzheimer's or other health condition. Such stand-alone panels are in some cases implemented as kits to be used in a medical or laboratory facility, or to be implemented by providing samples for analysis at a centralized facility.

In some cases, however, biomarkers retain predictive utility independent of any information regarding a protein from which they are derived. That is, biomarkers identified as mass spectrometric signals having levels that vary in correlation with the presence or severity of a health condition or health status may in some cases retain a utility as markers on their own. Even without information regarding a biological mechanism underlying the correlation (as may be obtained by identifying a protein correlating to the marker and by examining the biological function of the protein) the biomarker in itself, as it appears on the mass spectrometric result, possess utility as a biomarker alone or in combination as indicative of a health status or condition or level of severity. Such biomarkers often rely upon mass spectrometric detection and may not in all cases be conducive to development as immunologically based stand-alone assays. However, they remain useful as stand-alone markers or as constituents of detection approaches comprising mass spectrometry-based detection at least some biomarkers in a panel.

In some cases, even when a biomarker identity is not known, one can generate a labeled biomarker that migrates at a predicted offset relative to the unidentified relevant biomarker. Thus, even in the absence of the biomarker's identity, labeled offset biomarker approaches can be used to facilitate high-throughput collection of this type of marker.

Biomarker databases developed hereby often possess a number of interrelated characteristics. Firstly, the databases can harbor from less than 20 to 1,000s or 10,000s of biomarkers per sample, and often further comprise non-biomarker data such as glucose levels, age, caloric intake, sleep patterns, blood pressure measurements, mental acuity tests or other non-sample marker data as disclosed herein.

Accordingly, signals can be derived from these biomarker datasets by assembling individual biomarker and other markers into panel that provide statistical signals that are strong enough for medical relevance even when the individual markers do not on their own generate statistically relevant or medically reliable signals.

Secondly, biomarker databases developed herein are readily generated from easily obtained starting material. Samples yielding at least 10, at least 50, at least 100, at least 1,000, at least 5,000, at least 10,000, at least 20,000, at least 30,000 or more markers are obtained from dried blood spots or other blood stabilization approach such as sponge collection, and collected often remote from a medical or laboratory facility. Biomarkers are also readily obtained in substantial numbers from collected breath aspirate or from other fluid or tissue sample.

The adoption of such easily obtained starting material facilitates both generation of large numbers of biomarkers from a single sample, but also facilitates processing of multiple samples, either from multiple individuals in at least one cohort, or from a single individual over multiple points in a time course, or from multiple individuals at multiple time points. The ease with which samples are collected and processed exponentially increases dataset size.

Thirdly, because biomarker databases are generated readily from easily obtained and stored samples, because such large numbers of biomarkers are assayed from a single sample, and because samples are readily obtained from single individuals over multiple times of a time course, one is able to investigate changes over time in an individual's biomarker profile on a scale comparable to that of one's genomic or exomic nucleic acid sequence information, and at the same time to detect changes in this dataset indicative of changes in health status. Nucleic acid databases are valuable sources of personalized medical information, but are ill-suited to detect changes that occur over time, such as changes leading to mutations in genes implicated with changes in health status or health category. Cancer mutations, for example, often occur only in a tiny subset of cells in an individual. Untargeted genomic sequencing efforts do not detect these mutations at any reliable frequency. Thus, inherited oncogenes are readily detected, but changes that may impact health status are unlikely to be detected in a general genomic sequencing effort.

Using databases generated as disclosed herein, biomarkers are obtained having a level of information comparable to the relevant information in a genomic sequence (that is, comparable to the subset of genomic information that varies among individuals and is relevant to health status or health classification). However, additionally, as a genomic or other change occurs in an individual that may impact health status or health classification, these changes are readily detected in real time in the generation of ‘longitudinal’ or temporally iteratively sampled databases as disclosed herein. Thus, unlike comparable genomic databases, the biomarker databases as disclosed herein capture signals that are reflected in differential levels of protein or other biomarkers as these changes occur. Databases as disclosed herein are consistent with and compatible with genomic information, and genomic information can be included as marker information for databases as disclosed herein to be considered in making health status or health categorization determinations, but unlike genomic data in isolation, biomarker databases as disclosed herein incorporate temporal information relating to health status or health condition progressions over time, such that one can identify not only a risk of developing a health condition, but to identify the condition in its early stages of development, thereby facilitating early treatment precisely when it is suitable for a given condition.

### Biomarker Database Uses

Biomarker databases as disclosed herein have at least two related uses in health assessment. Firstly, databases are used to identify markers that correlate with health status in among two cohorts that vary in that health status. Cohorts can comprise single sample marker information, or more often, marker data including biomarker data obtained from multiple members of each of at least two cohorts, sharing at least one common health status within each cohort. Biomarker or other markers that correlate with health status or health categorization, either alone or in combination, are identified from among the at least 10, at least 50, at least 100, at least 1,000, at least 5,000, at least 10,000, at least 20,000, at least 30,000 or greater than 30,000 biomarkers in the database. Biomarkers or other markers may be effective distinguishers of the cohorts alone, or more often in combination with other biomarkers or other markers to form panels that generate signals of stronger statistical relevance or predictive AUC values.

Biomarkers may correlate to or map to proteins of known function in a health status or health condition, or may correlate or map to proteins of unknown function. Alternately, biomarkers in some cases are not mapped to a known protein but are nonetheless useful as mass spectroscopy-based markers or distinguishers of health status or health category. Biomarkers of particular interest may subsequently be mapped to a protein, without impacting the biomarker's use in mass spectrometric analysis.

Biomarkers that are mapped to a particular protein are in some cases developed as health status or condition specific panels. These panels are consistent with mass spectrometric information, but in some cases are developed for independent, targeted use, for example in immunological assays. These assays are implemented through the use of stand-alone kits comprising immunological reagents for the detection of biomarker proteins, or through the delivery of samples to a facility for sample analysis.

Secondly, databases as disclosed herein are used for the ongoing temporal monitoring of at least one individual from whom database samples are obtained. In this use, an individual or individuals (such as an individual or a cohort of individuals subjected to a common treatment regimen, or a single individual or cohort of individuals for which no health status hypothesis is initially present) are subjected to ongoing sampling, and databases are developed ‘longitudinally’ or over time. Changes in biomarker levels are observed over time, and when the biomarkers are mapped to proteins implicated in at least one particular health condition or health status, the health status or health condition is identified as potentially being in flux in the individual or population. These uses are not mutually exclusive. Some databases are readily used for both aims. A significant change between measurements may comprise a change of at least 10%, or at least 1%, 2%, 5%, 10%, 20%, or at least 50% of a marker implicated in a disorder. A significant change between measurements may comprise a change of at least 10%, or at least 1%, 2%, 5%, 10%, 20%, or at least 50% of a plurality of markers implicated in a common disorder.

Additionally, databases are in some cases used to cluster patients into groupings independent of any current condition or category. Patients are grouped largely or solely based upon biomarker profiles, and are then observed for commonalities both at the time of sample collection, and retrospectively over time. As a health condition changes in a member of a given grouping, the remaining members of the grouping may be cautioned to assay for the health condition. Alternately, the member may have his or her biomarker profile reassessed, so as to determine whether the individual remains in said grouping.

Ongoing monitoring using the disclosure herein is implemented through a number of approaches, such as the following. An ongoing health monitoring protocol is implemented for an individual by measuring biomarkers from a wide diversity of potential sources, as indicated in FIG. 16. In some examples, biomarker data sources include physical data, personal data and molecular data. In some examples, physical data sources include but are not limited to blood pressure, weight, heart rate, and/or glucose levels. In some examples, personal data sources include cognitive well-being. In some examples, molecular data sources include but are not limited to specific protein markers. In some examples, molecular data includes mass spectrometric data obtained from plasma samples obtained as dried blood spots and/or obtained from captured exudates in breath samples. One example of raw mass spectrometric data generated from captured exudates in breath is given in FIG. 17. In some examples, biomarker and other marker data from multiple sources are integrated as part of a multi-source marker regimen, and depicted in FIG. 18.

Data is collected and analyzed over time. Groups of markers that change over time and are linked may be monitored together, for example, markers implicated in glucose regulation such as glucose levels, mental acuity, and patient weight. In some examples, differences in these markers may be indicative of disease states or disease progression. For example, glucose levels are found to vary over the course of the protocol. Glucose levels are observed to be successively less regulated, but not at levels that would on their own indicate diabetes. Biomarkers correlating to glucose regulation, and implicated in diabetes, are found to change in levels monitored through the course of the monitoring. It is observed that mental acuity is affected in a manner that correlates with blood glucose levels. It is also observed that the magnitude of these changes scales roughly with an increase in patient weight. In this example, each of these markers shows some change, but none of these markers individually generates a signal strong enough to lead to a statistically significant signal indicative of progression toward diabetes. Nonetheless, the aggregate signal generated by a multifaceted analysis involving markers from a diversity of sources, including biomarkers from patient dried blood samples, strongly indicates a pattern trending toward the onset of diabetes.

### Labeled Biomarker Reference Molecules

Some mass spectrometric or other approaches herein involve labeled biomarker reference molecules or standards, variously referred to as mass markers, reference markers, labeled biomarkers, or otherwise referred to herein. Such standards or labeled biomolecules facilitate native biomarker identification, for example in automate, high throughput data acquisition. A number of reference molecules are consistent with the disclosure herein.

Reference biomarker molecules are optionally isotopically labeled, such as using at least one of H2, H3, heavy nitrogen, heavy carbon, heavy oxygen, S35, P33, P32, and isotopic selenium. Alternately or in combination, reference biomarker molecules are chemically modified, such as using at least one of oxidized, acetylated, de-acetylated, methylated, and phosphorylated or otherwise modified to produce a slight but measurable change in overall mass. Alternately or in combination, reference biomarker molecules are nonhuman homologs of human proteins in the biomarker set.

A characteristic common to reference biomarkers include a repeatable offset co-migration with the native biomarker, such that the reference biomarker migrates near but not exactly with the biomarker of interest. Thus, detection of the biomarker is indicative that the native marker should be present at a predictable offset from the labeled biomarker.

A second characteristic common to some biomarker references is that they are readily identifiable in mass spectrometric data output. Often, biomarkers are identified in mass spectrometric output because their mass and therefore their position are precisely known in mass spectrometric output. By calculating their expected position and looking for a spot at that position having an expected concentration or signal, one can identify labeled markers in mass spectrometric output.

Mass-based identification of marker polypeptides are is optionally further facilitated using any one or more of the following approaches. Firstly, an identified marker or marker set is run on its own, in the absence of a sample, so as to identify experimentally the exact positions where the markers run for a given mass spectrometric analysis. The markers are then run with the sample, and results are compared so as to identify the marker positions. This is done, for example, by overlaying results of one run involving only marker polypeptides with results of a second run comprising both marker polypeptides and sample biomarkers.

Secondly, various aliquots of the sample are provided with different concentrations of marker polypeptides. Mass spectrometric data for each of the marker dilution concentration variants are analyzed. Sample spots are expected (and observed) to show a high repeatability in spot location and intensity. Marker polypeptides, in contrast, show a high repeatability in spot location but a predictable variation in spot intensity that correlates with the concentration of marker added.

Thirdly, marker polypeptides are identified by their location on mass spectrometric outputs, and their identity is confirmed by the detection of a corresponding native protein or polypeptide at a predicted offset position, such that they indicate the presence of their native marker not by an independent signal but by presence as a ‘doublet’ having a predicted offset in a mass spectrometric output. This approach relies upon the native protein or polypeptide being present in the sample, but as this is often the case, the approach is valuable for the majority of the markers.

These approaches are not mutually exclusive. For example, one may generate a mass spectrometric output that only includes markers, and overlay that result against multiple sample mass spectrometric analyses having varying marker concentrations so as to identify markers at the expected locations and exhibiting the expected variation in spot signal strength relative to other runs. Independently or in combination with either of the approaches, one searches the mass spectrometric data to identify native spots at the expected offset from putative marker spots, thereby coming to finalized marker spot calls.

Alternately, identification is accomplished by heavy isotope radiolabeling. Such reference biomarkers are labeled consistent with mass spectrometric visualization, but are independently detectable through radiometric approaches, so as to facilitate their detection independent of the detection signal for native biomarkers in the sample.

Heavy isotope labeling, is particularly useful because it provides a predictable size-offset to facilitate native spot identification. However, other reference molecule labeling approaches are consistent with the disclosure herein.

Most often, a protein that yields a biomarker of interest is identified, and a reference biomarker is generated therefrom. Such protein biomarker reference molecules are, for example, synthesized with a detectable isotope of hydrogen, carbon, nitrogen, oxygen, sulfur or in some cases phosphate or even selenium. Reference biomarkers that are generated from synthetic versions of biomarkers of interest are beneficial because, aside from the mass offset, they are expected to behave comparably to native proteins in mass spectrometric analysis.

Alternately, non-protein biomarkers are used in some cases. Non-protein biomarkers have the advantage of often being simpler to synthesize. Additionally, one does not need the identity of the biomarker of interest to develop a non-protein biomarker. Rather, any labeled non-protein biomarker that migrates repeatably with a predictable offset from a biomarker of interest is consistent with the disclosure herein.

Aside from their role in marking or facilitating identification of native polypeptides, labeled reference markers are also useful in relative quantification of identified polypeptide spots on a mass spectrometric output. Labeled reference markers are introduced to a sample at known concentrations, and their signals in the mass spectrometric output are indicative of these concentrations. Spots corresponding to native proteins in the mass spectrometric output are readily and accurately quantified by comparing mass spectrometric signal strength to reference polypeptides of known concentration.

In some cases, two, more than two, up to 10%, 20%, 30%, 40%, 50%, 75%, 90%, up to all labeled reference markers are added at a single concentration, facilitating assessment of signal variation across polypeptide sizes and positions in the mass spectrometric output. Alternately or in combination, marker proteins or polypeptides are introduced at varying concentrations, such that one can compare a native mass spectrometric spot to a plurality of marker spots at varying intensities, thereby more accurately correlating a native spot signal to a reference signal of known concentration or amount. In some cases, various sets of marker proteins are introduced at a first concentration, while various other sets are introduced at other concentrations, thereby accomplishing both of the above-mentioned benefits. That is, markers at a common concentration or amount facilitate identification of variation in signal among markers and native mass spectrometric spots, while markers at a varying concentrations or amounts allow one to match native mass spectrometric spots to a spot of known amount or concentration across a broad range of amounts or concentrations, thereby providing an accurate reference for quantification of native mass spectrometric spots, and ultimately of native marker proteins or polypeptides, in a sample.

### Assessing Biomarker Signals

Biomarkers, either individual or collective biomarkers assembled into panels of at least two biomarkers, are assessed as to their significance as to patient health. A number of panel assessment approaches are consistent with the disclosure herein. Furthermore, additional approaches not explicitly recited herein are nonetheless consistent with the disclosure herein and there incorporation into a method or system is not inconsistent with the method of system falling within the scope of claims issuing from this disclosure.

Biomarker panel levels are obtained and assessed through at least one of the following approaches in various embodiments disclosed herein. In relatively simple cases, biomarker panel levels are compared to a reference level measured from an individual of known condition, and the patient is determined to share the condition if the biomarker levels do not differ significantly from the reference. Statistical assessment of whether or not two panels ‘differ significantly’ is made through any number of well-known or innovative approaches.

A number of methods of determining if one set of values differs significantly from another set of values are available. Such statistical tests (e.g., Analysis of Variation (ANOVA), t-tests, and Chi-squared analyses) are routine and have been for some time in the field of biological statistical analysis. Alternately, panel levels are evaluated using more elaborate computational approaches, such as machine learning or neural networking approaches.

Such tests, or other statistical tests known to one of skill in the art, are sufficient for assessing whether an increase, decrease, equal amount, numerical expression of standard deviations or some other protocol differs from a control reference set of values so as to warrant the classification of a measured set of panel values as differing substantially from a control set.

A person of ordinary skill in the art understands that they are directed to performing an appropriate statistical test to determine whether a measures set of values differs significantly from one or more reference sets of values.

For example, a person of ordinary skill in the art may wish to compare the accumulation levels of the proteins in a protein panel to a standard range derived from a plurality of reference samples. In such a situation, a person of ordinary skill in the art recognizes that a z-statistic or a t-statistic, for example, is an appropriate metric. A z-statistic makes use of the known reference population mean and variance to determine the probability that a sample drawn from the reference population would exhibit a more extreme measurement than a given cut-off. Cut-off values are determined such that a measurement more extreme than the cut off has a low probability (i.e., p-value) of being chosen from the reference population.

Furthermore, a person of ordinary skill in the art understands that a determination of statistically significant difference can be made using, for example, a t-test to determine the probability that their measurements could be provided by a reference sample. A person of ordinary skill in the art further recognizes that assessing the p-value cut-off depends on the application of the test results. Certain results, at a medical practitioner's or other user's discretion, may warrant more stringent evaluation of ‘significant’ that would otherwise be necessary.

For example, if the purpose of the test is to determine which patients receive a non-invasive, low-risk follow-up procedure, a relatively high p-value cut-off (e.g. p-value<0.1) might be select as a relatively high number of false positives will have little consequence. On the other hand, if the application of the test is a surgical or chemotherapeutic intervention, a much more stringent cutoff will likely be required to ensure a higher specificity. These considerations are well-understood and routine in the fields of epidemiology and medical test design.

Alternately or in combination, panel measurements are evaluated as to whether they pass a threshold at which a health status assessment is expected to change. That is, rather than, or in addition to scoring deviation from a reference panel value set or range, one assesses whether panel values, individually or collectively, surpass a threshold so as to constitute a change in health status assessment. In some cases the threshold is a sharp distinguisher between health status categories. Alternately, in some cases panels near a threshold are ‘not called,’ so that they are not categorized with confidence in either health category. Such a categorization strategy increases the confidence of categorization calls that are made, but leaves some panels uncategorized.

Alternately or in combination, samples are scored not by a binary yes/no categorization, but are assigned a percentile value relative to the reference database. The percentile value indicates, for example, where the sample measurements fit along a linear scale of the measurements or values of the database, such that one may determine from the analysis whether the sample values are typical of the reference dataset, or are outliers.

A number of approaches are available for fitting reference values on a linear scale relative to one another, and assigning a percentile value to a sample relative to the reference value. For example, reference values may be assessed on a marker by marker basis to determine mean or median values, and then sorted on a marker by marker basis as to how greatly the differ from the mean or median values. Rankings on a marker by marker bases are then assessed, for example, averaged or assigned statistical assessments of deviation from a mean or median value set (standard deviation determination, Chi-squared analysis, ANOVA, and other analyses are consistent with this approach), to determine which sample marker sets or panels, on a by marker basis or in aggregate, differ most substantially from the mean or median values per marker or in aggregate. A similar analysis is performed in a sample to be categorized, so as to assess the sample relative to the reference database. A number of alternative approaches to sample panel categorization are known in the art and consistent with the disclosure herein.

Similarly, a broad range of reference sets are consistent with the disclosure herein. As discussed above, some reference sets involve a single measurement, such as a single measurement of panel values from a single individual taken at a single time point. Such a measurement is optionally taken from a reference individual of known health status for the condition or status assessed by the panel, such that a substantially similar panel set indicates a common condition status. The reference individual is optionally a healthy individual or an individual suffering from a condition assayed by a panel, and may have any of a number of varying levels of severity of the condition. In some cases the reference panel is taken from the individual whose health is being assessed, but was obtained when a certain health condition was known (or later verified through ongoing health monitoring), so that difference from the level indicates a change in the individual.

Reference sets comprising more than one set of panel measurements are also consistent with the disclosure herein. Reference sets are generated from a plurality of individuals, such as 2, 3, 4, 5, 6, 7, 8, 9, 10, 20, 50, 100, 200, 500, 1000, 2000, 5000, 10,000, or more than 10,000 individuals, or a number comparable to a number listed herein. Preferably, the individuals share a common health status, and may in some cases be further sorted by a level of severity if their health status is positive for a condition having varying levels of severity. Alternately or in combination, reference sets are derived from multiple samples taken over time from at least one individual, such as the individual for which a later health assessment is to be made. Also contemplated are ‘two-dimensional’ reference sets, comprising panel information obtained from at least two individuals from at least two time points for some or all of the individuals.

When references comprise multiple panel sets, the references variously represent ranges of panel levels and panel constituent levels consistent with the health status of the reference. Thus, by using a multi-measurement panel, one is able to determine ranges of values consistent with a given health status, so as to assess whether an individual's panel levels fall within said ranges, do not differ significantly from said ranges, or do differ significantly from said ranges, so as to assess whether the individual warrants categorization as having the health status. Drawing from multiple panels provides for a representation of variation within panel levels consistent with a health categorization. Accordingly, one of skill in the art may tailor assessment statistical stringency to panel reference, such that assessment against references comprising multiple panels are given a higher degree of confidence at a given level of variation, relative to the same variation between a measured panel and a reference constructed from a single set of panel data.

Health conditions for which a reference set are developed include diseases as conventionally contemplated, such as various cancers, renal health, cardiovascular health, brain health, neuromuscular health or presence of a communicable disease. Alternately, more generalized ‘conditions’ are assessed by comparison to a reference, such as age, energy level, alertness, or other status. In such cases, an individual is assessed as to whether the individual presents a panel level consistent with the individual's chronological age, or whether the individual possesses panel information consistent with references of another age group.

### Machine Learning

Some embodiments involve machine learning as a component of database analysis, and accordingly some computer systems are configured to comprise a module having a machine learning capacity. Machine learning modules comprise at least one of the following listed modalities, so as to constitute a machine learning functionality.

Modalities that constitute machine learning variously demonstrate a data filtering capacity, so as to be able to perform automated mass spectrometric data spot detection and calling. This modality is in some cases facilitated by the presence of marker polypeptides, such as heavy isotope labeled polypeptides or other markers in a mass spectrometric analysis output, so that native peptides are readily identified and in some cases quantified. The markers are optionally added to samples prior to proteolytic digestion or subsequent to proteolytic digestion. Markers are in some embodiments present on a solid backing onto which a blood spot or other sample is deposited for storage or transfer prior to analysis via mass spectroscopy.

Modalities that constitute machine learning variously demonstrate a data treatment or data processing capacity, so as to render called data spots in a form conducive to downstream analysis. Examples of data treatment include but are not necessarily limited to log transformation, assigning of scaling ratios, or mapping data to crafted features so as to render the data in a form that is conducive to downstream analysis.

Machine learning data analysis components as disclosed herein regularly process a wide range of features in a mass spectrometric data set, such as 1 to 10,000 features, or 2 to 300,000 features, or a number of features within either of these ranges or higher than either of these ranges. In some cases, data analysis involves at least 1 k, 2 k, 3 k, 4 k, 5 k, 6 k, 7 k, 8 k, 9 k, 10 k, 20 k, 30 k, 40 k, 50 k, 60 k, 70 k, 80 k, 90 k, 100 k, 120 k, 140 k, 160 k, 180 k, 200 k, 220 k, 2240 k, 260 k, 280 k, 300 k, or more than 300 k features.

Features are selected using any number of approaches consistent with the disclosure herein. In some cases, feature selection comprises elastic net, information gain, random forest imputing or other feature selection approaches consistent with the disclosure herein and familiar to one of skill in the art.

Selected feature are assembled into classifiers, again using any number of approaches consistent with the disclosure herein. In some cases, classifier generation comprises logistic regression, SVM, random forest, KNN, or other classifier approaches consistent with the disclosure herein and familiar to one of skill in the art.

Machine learning approaches variously comprise implementation of at least one approach selected from the list consisting of ADTree, BFTree, ConjunctiveRule, DecisionStump, Filtered Classifier, J48, J48Graft, JRip, LADTree, NNge, OneR, OrdinalClassClassifier, PART, Ridor, SimpleCart, Random Forest and SVM.

Applying machine learning, or providing a machine learning module on a computer configured for the analyses disclosed herein, allows for the detection of relevant panels for asymptomatic disease detection or early detection as part of an ongoing monitoring procedure, so as to identify a disease or disorder either ahead of symptom development or while intervention is either more easily accomplished or more likely to bring about a successful outcome. Monitoring is often but not necessarily performed in combination with or in support of a genetic assessment indicating a genetic predisposition for a disorder for which a signature of onset or progression is monitored. Similarly, in some cases machine learning is used to facilitate monitoring of or assessment of treatment efficacy for a treatment regimen, such that the treatment regimen can be modified over time, continued or resolved as indicated by the ongoing proteomics mediated monitoring.

Machine learning approaches and computer systems having modules configured to execute machine learning algorithms facilitate identification of classifiers or panels in datasets of varying complexity. In some cases the classifiers or panels are identified from an untargeted database comprising a large amount of mass spectrometric data, such as data obtained from a single individual at multiple time points, samples taken from multiple individuals such as multiple individuals of a known status for a condition of interest or known eventual treatment outcome or response, or from multiple time points and multiple individuals.

Alternately, in some cases machine learning facilitates the refinement of a panel through the analysis of a database targeted to that panel, by for example collecting panel information for that panel from a single individual over multiple time points, when a health condition for the individual is known for the time points, or collecting panel information from multiple individuals of known status for a condition of interest, or collecting panel information from multiple individuals at multiple time points. As is readily apparent, in some cases collection of panel information is facilitated through the use of mass markers, such as heavy-labeled or ‘light-labeled’ mass markers that migrate so as to identify nearby unlabeled spots corresponding to the marked polypeptides. Thus, panel information is collected either alone or in combination with untargeted mass spectrometric data collection. Panel data is subjected to machine learning, for example on a computer system configured as disclosed herein, so as to identify a subset of panel markers that either alone or in combination with one or more non-panel markers analyzed through an untargeted approach, account for a health status signal. Thus, machine learning in some cases facilitates identification of a panel that is individually informative of a health status in an individual.

### Dried Blood Spot Analysis

Methods, databases and computers configured to receive mass spectrometric data as disclosed herein often involve processing mass spectrometric data sets that are spatially, temporally or spatially and temporally large. That is, datasets are generated that in some cases comprise large amounts of mass spectrometric data points per sample collected, are generated from large numbers of collected samples, and are in some cases generated from multiple samples derived from a single individual.

Data collection is in some cases facilitated by depositing samples such as dried blood samples (or other readily obtained samples such as urine, sweat, saliva or other fluid or tissue) onto a solid framework such as a solid backing or solid three-dimensional framework. The sample such as a blood sample is deposited on the solid backing or framework, where it is actively or passively dried, facilitating storage or transport from a collection point to a location where it may be processed.

As disclosed herein, a number of approaches are available for recovering proteomic or other biomarker information from a dried sample such as a dried blood spot sample. In some cases samples are solubilized, for example in TFE, and subjected to proteolysis to generate fragments to be visualized by mass spectrometric analysis. Proteolysis is accomplished by enzymatic or non-enzymatic treatment. Exemplary proteases include trypsin, but also enzymes such as proteinase K, enteropeptidase, furin, liprotamase, bromelain, serratipeptidase, thermolysin, collagenase, plasmin, or any number of serine proteases, cysteine proteases or other specific or nonspecific enzymatic peptidases, used singly or in combination. Nonenzymatic protease treatments, such as high temperature, pH treatment, cyanogen bromide and other treatments are also consistent with some embodiments.

When particular mass spectrometric fragments are of interest or use in analysis, such as a biomarker panel indicative of a health condition status, it is often beneficial to include heavy-labeled or other markers as standard markers as described herein. Markers, as discussed, migrate on a mass spectrometric output at a known position and at a known offset relative to the sample fragments of interest. Inclusion of these markers often leads to ‘offset doublets’ in mass spectrometric output. By detecting these doublets, one can readily, either personally or through an automated data analysis workflow, identify particular spots of interest to a health condition status among and in addition to the full range of mass spectrometric output data. When the markers have known mass and amount, and optionally when the amount loaded into a sample varies among markers, the markers are also useful as mass standards, facilitating quantification of both the marker-associated fragments and the remaining fragments in the mass spectrometric output.

Standard markers are introduced to a sample either at collection, during or subsequent to resolubilization, prior to digestion or subsequent to digestion. That is, in some cases a sample collection structure such as a solid backing or a three-dimensional volume is ‘pre-loaded’ so as to have a standard marker or standard markers present prior to sample collection. Alternately, the standard markers are added to the collection structure subsequent to sample collection, subsequent to sample drying on the structure, during or subsequent to sample collection, during or subsequent to sample resolubilization, or during or subsequent to sample proteolysis treatment. In preferred embodiments, exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 160, 170, 180, 190, 200, 225, 250, 275, 300, or more than 300 standard markers are added to a collection structure prior to sample collection, such that standard processing of the sample results in a mass spectrometric output having the standard markers included in the output without any additional processing of the sample. Accordingly, some methods disclosed herein comprise providing a collection device having sample markers introduced onto the surface prior to sample collection, and some devices or computer systems are configured to receive mass spectrometric data having standard markers included therein, and optionally to identify the mass spectrometric markers and their corresponding native mass fragment.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

“About” a number, as used herein, refers to a range including that number and spanning that number plus or minus 10% of that number. “About” a range refers to the range extended to 10% less than the lower limit and 10% greater than the upper limit of the range.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB.NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of biomarker information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Discussion of the Accompanying Figures

At FIG. 1 one sees an exemplary Noviplex DBS plasma card having an overlay, a spreading layer, a separator, a plasma collection reservoir, an isolation screen, and a base card. Whole blood is applied to a spot on the overlay where it reaches the spreading layer and the separator which allows the plasma to pass through to the plasma collection reservoir. At FIG. 2 one sees 48 mass spectrometry output graphs resulting from 16 samples subjected to three mass spectrometry runs. MS1 data images from 48 injections of a technical replicate variability study are presented. The 16 DBS cards are shown in the columns with their technical replicates in the rows. For each individual MS1 image, the horizontal axis is m/z and the vertical axis is LC time. To show a high-level view of the data quality and reproducibility, a visual representation of the MS1 data from a repeated sampling experiment is shown. Here, each image in the grid shows the data from a single injection on LC time vs. m/z axes, with the color scale representing signal abundance (from black-no signal, to red-high signal). The consistency of the images shows the repeatability of the assay.

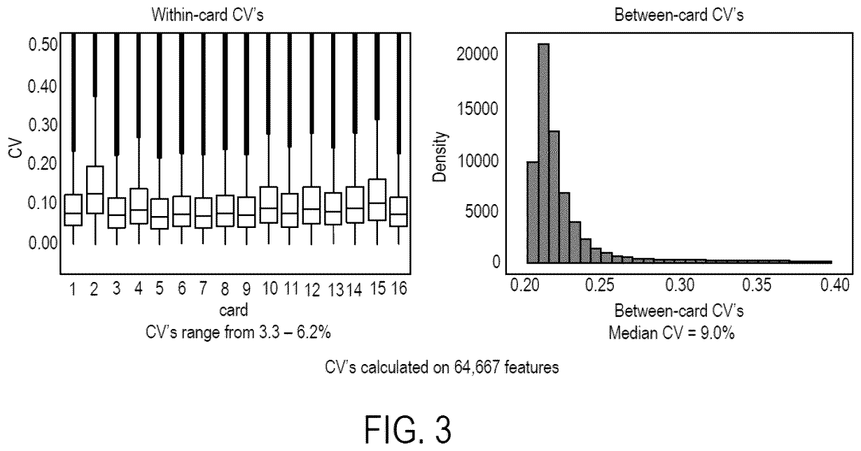

At FIG. 3 left panel one sees within card coefficients of variation (CV) with the CV on the Y axis and each DBS card on the X axis. CVs range from 3.3 to 6.2%. At FIG. 3 right panel one sees between card CV with the density on the Y axis and the between card CV on the X axis. The median CV was found to be 9.0%. CV was calculated on 64,667 features.

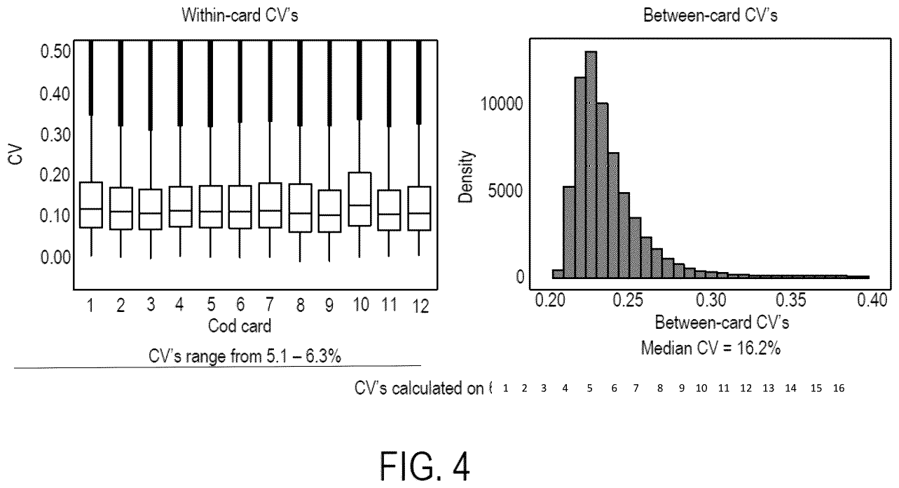

At FIG. 4 left panel one sees within card coefficients of variation (CV) with the CV on the Y axis and each DBS card on the X axis. CVs range from 5.1 to 6.3%. At FIG. 3 right panel one sees between card CV with the density on the Y axis and the between card CV on the X axis. The median CV was found to be 16.2%. CV was calculated on 65,795 features.

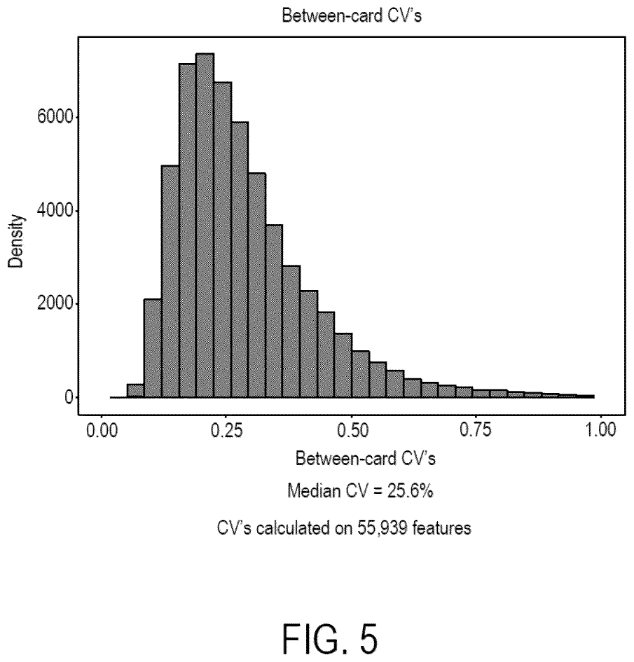

At FIG. 5, one sees between-card coefficient of variation (CV) with the density on the Y axis and the between card CV on the X axis. The median CV was 25.6% and CVs were calculated on 55,939 features.

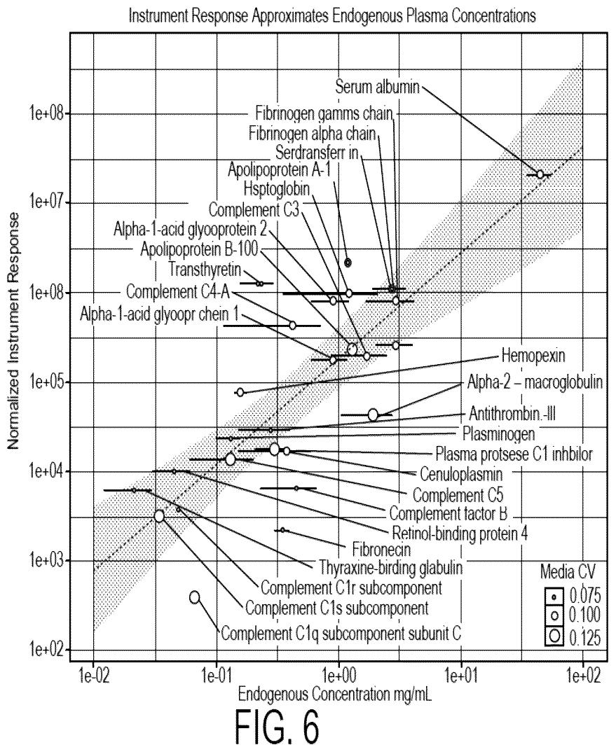

At FIG. 6 one sees a graph illustrating that instrument response is approximating endogenous plasma concentration. This graph has an X axis with the measurement of endogenous concentration and a Y axis with a normalized instrument response. Each protein is labeled with the protein name and a spot sized to the median CV with the smallest size having a median CV of 0.075, the medium size having a median CV of 0.100, and the largest size having a median CV of 0.125. A dashed line shows a perfect correlation and the shaded area shows modest variation from the perfect correlation.

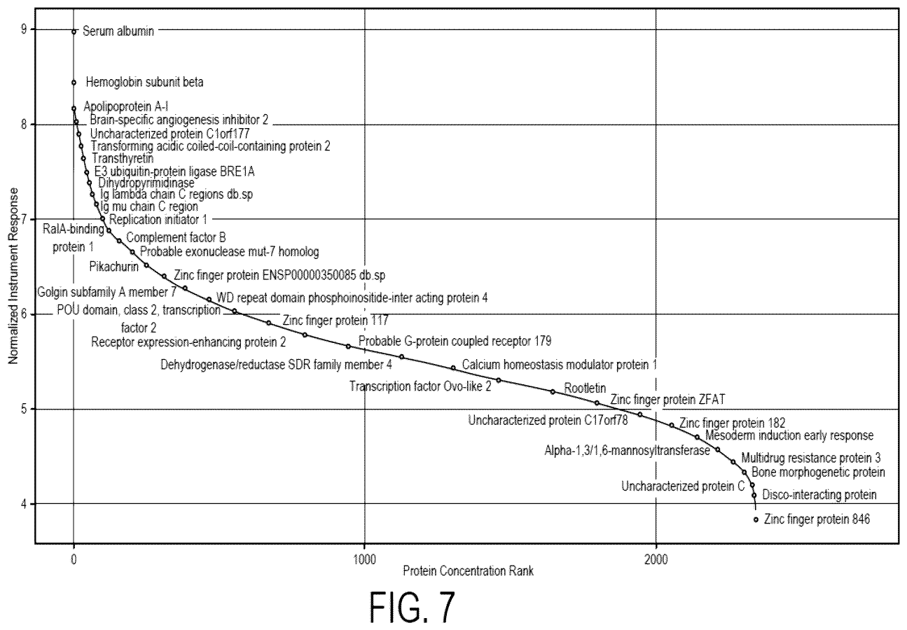

At FIG. 7 one sees a graph of the normalized instrument response versus the protein concentration rank. Proteins are ranked by protein concentration ordered on the X axis from greater to lesser concentration. The normalized instrument response is on the Y axis.

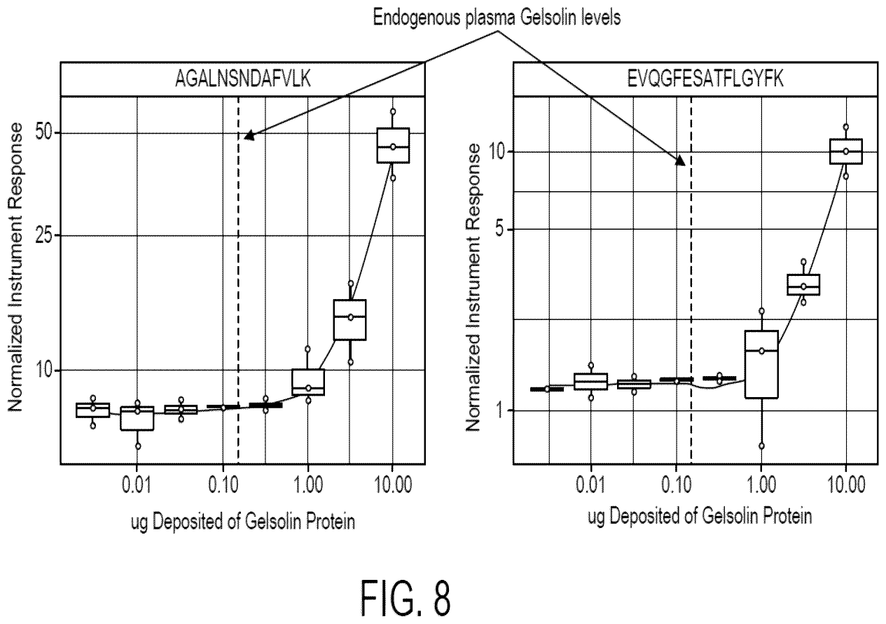

At FIG. 8 one sees endogenous plasma gelsolin levels measured using two peptides. Each graph has an X axis of ug deposited gelsolin protein and a Y axis of normalized instrument response. The left panel uses a peptide with a sequence AGALNSNDAFVLK and the right panel uses a peptide with a sequence EVQGFESATFLGYFK.

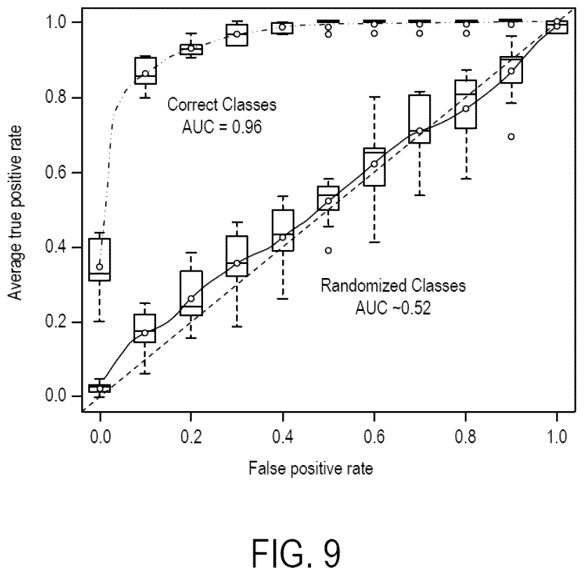

At FIG. 9 one sees the results of prediction of sex of the sample of origin. Two curves are shown on a graph with an X axis of false positive rate and a Y axis of average true positive rate. Correct classes are shown in the top curve with an AUC of 0.96 and randomized classes are shown in the bottom curve with an AUC of approximately 0.52.

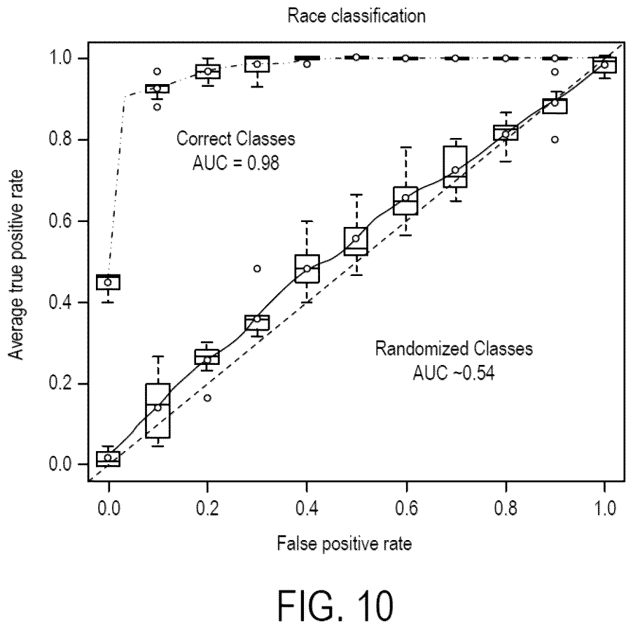

At FIG. 10 one sees the results of prediction of race of the sample of origin. Two curves are shown on a graph with an X axis of false positive rate and a Y axis of average true positive rate. Correct classes are shown in the top curve with an AUC of 0.98 and randomized classes are shown in the bottom curve with an AUC of approximately 0.54.

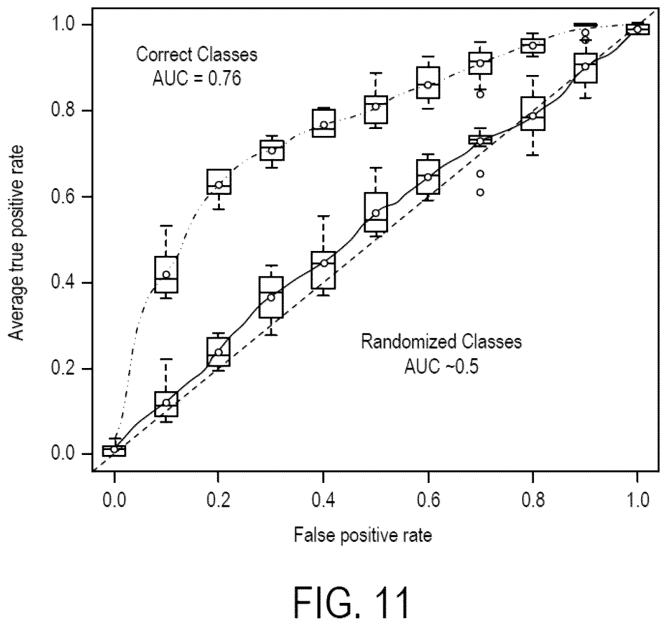

At FIG. 11 one sees the results of prediction of colorectal cancer (CRC) status of the sample of origin. Two curves are shown on a graph with an X axis of false positive rate and a Y axis of average true positive rate. Correct classes are shown in the top curve with an AUC of 0.76 and randomized classes are shown in the bottom curve with an AUC of approximately 0.5.

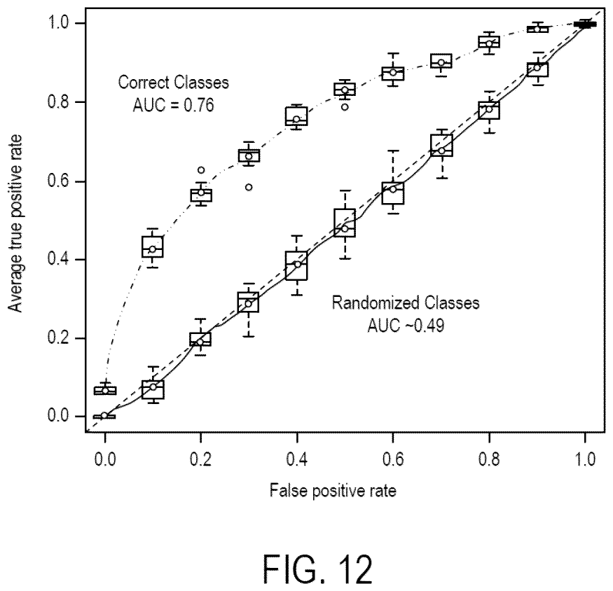

At FIG. 12 one sees the results of prediction of colorectal cancer (CRC) status of the sample of origin. Two curves are shown on a graph with an X axis of false positive rate and a Y axis of average true positive rate. Correct classes are shown in the top curve with an AUC of 0.76 and randomized classes are shown in the bottom curve with an AUC of approximately 0.49.

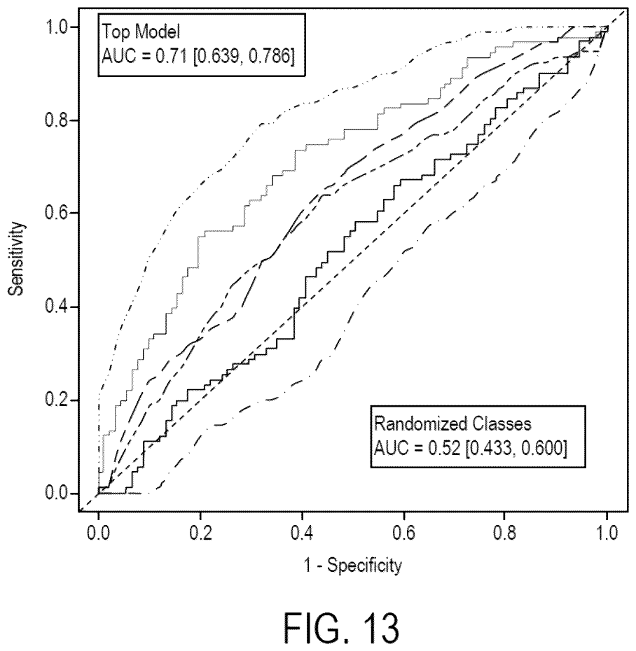

At FIG. 13 one sees the results of prediction of coronary artery disease (CAD) status of the sample of origin. Two curves are shown on a graph with an X axis of specificity and a Y axis of sensitivity. Each curve has an error curve above and below the curve. Correct classes are shown in the top curve with an AUC of 0.71 and randomized classes are shown in the bottom curve with an AUC of 0.52. One sees that the curves and their error bars do not overlap and are distinct.

At FIG. 14 one sees two graphs of an LC gradient (left panel) and an optimized gradient (right panel. Each graph has a percent organic depicted on the Y axis and chromatography time depicted on the X axis. A linear portion of the plot is highlighted with a square.

At FIG. 15 one sees a mass spectrometric analysis of a 30 minute gradient (left panel) and a 10 minute gradient (right panel). The left panel shows approximately 30,000 features per sample with a z=2-4. The right panel shows greater than 10,000 features per sample with a z=2-4.

At FIG. 16 one sees various sources of biomarker data including physical data such as blood pressure, weight, blood glucose; personal data such as cognitive well-being and heart rate; and molecular data collected from blood plasma and breath.

At FIG. 17 one sees an exemplary tube for collecting breath as well as VOCs analyzed by mass spectrometry from a breath sample. This figure demonstrates that meaningful biomarker data can be collected from breath.

At FIG. 18 one sees an exemplary data collection scheme of data from 30-50 individuals with data collected weekly for 12-16 weeks. Collected data include molecular profiling via DPS and breath condensate; activity profiling such as calories, blood pressure, heart rate, and weight; and personal data profiling via mood and health. These data are compiled and analyzed in an exemplary graph of blood glucose plotted each day.

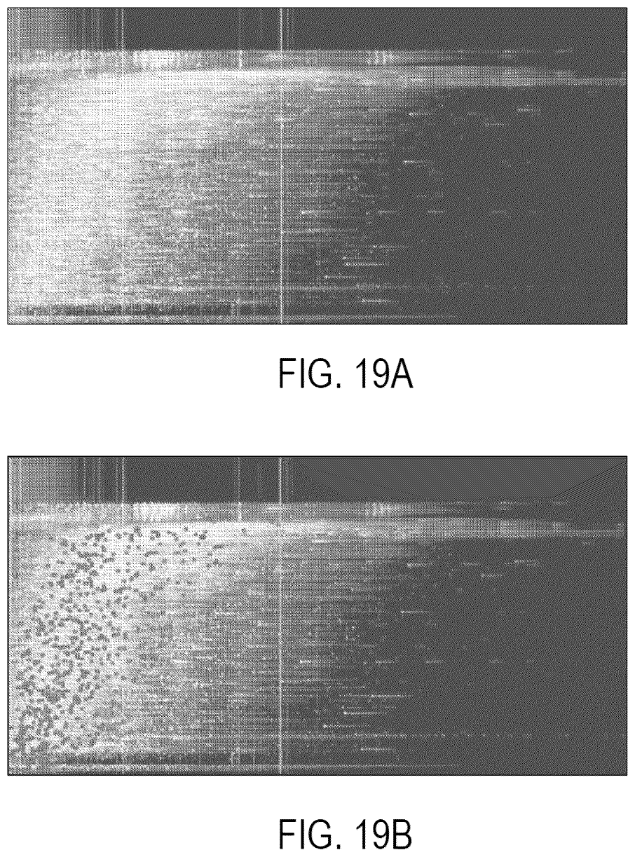

At FIG. 19A one sees output data of a mass spectrometric analysis showing more than 10,000 spots. At FIG. 19B one sees output data of a mass spectrometric analysis as in FIG. 19A with an overlay of positions of added heavy labeled markers depicted as red dots in the graph. These two figures in combination demonstrate how reference markers facilitate identification of native spots in mass spectrometric output.

At FIG. 20 one sees results of a representative list of 16 markers. Each graph shows marker concentration on the X axis and spot signal intensity on the Y axis. Spot calls determined to be accurate are depicted as filled circles having black outlines. Spot calls determined to be miscalled are depicted as light grey without an outline.

### Numbered Embodiments

1. A method of assessing a health status in an individual comprising obtaining a first measurement of a set of at least 10 fluid-borne markers from the individual at a first time point; obtaining a second measurement of the set of at least 10 markers at a second time point; and comparing the first measurement of the set of at least 10 markers to the second measurement of the set of at least 10 markers; and identifying said health status in said individual as changed when said comparing indicates a significant change between said first measurement and said second measurement. 2. The method of embodiment 1, or any of the above embodiments, wherein obtaining the first measurement comprises performing at least one immunoassay on a fluid sample from the individual. 3. The method of embodiment 1, or any of the above embodiments, wherein obtaining the first measurement comprises performing at least one immunoassay on a blood sample from the individual. 4. The method of embodiment 1, or any of the above embodiments, wherein obtaining the first measurement comprises performing at least one mass spectrometric assay on a fluid sample from the individual. 5. The method of embodiment 1, or any of the above embodiments, wherein obtaining the first measurement comprises performing at least one mass spectrometric assay on a blood sample from the individual. 6. The method of embodiment 1, or any of the above embodiments, wherein obtaining the first measurement comprises volatilizing a dried fluid sample. 7. The method of embodiment 1, or any of the above embodiments, wherein obtaining the first measurement comprises volatilizing a dried blood sample. 8. The method of embodiment 1, or any of the above embodiments, wherein the second measurement is taken from a sample obtained from the individual. 9. The method of claim 1, or any of the above embodiments, wherein the second measurement is obtained from a set of reference circulating markers. 10. The method of claim 1, or any of the above embodiments, wherein the set of at least 10 markers comprises markers selected to provide information related to a particular disorder. 11. The method of claim 1, or any of the above embodiments, wherein the set of at least 10 markers comprises markers selected to provide information related to a plurality of disorders. 12. The method of claim 1, or any of the above embodiments, wherein the set of at least 10 markers does not comprise markers selected to provide information related to a particular disorder. 13. The method of embodiment 1, or any of the above embodiments, wherein the set of at least 10 markers comprises at least 20 markers. 14. The method of embodiment 1, or any of the above embodiments, wherein the set of at least 10 markers comprises at least 30 markers. 15. The method of embodiment 1, or any of the above embodiments, wherein the set of at least 10 markers comprises at least 50 markers. 16. The method of embodiment 1, or any of the above embodiments, wherein the set of at least 10 markers comprises at least 100 markers. 17. The method of embodiment 1, or any of the above embodiments, wherein the set of at least 10 markers comprises at least 200 markers. 18. The method of embodiment 1, wherein the set of at least 10 markers comprises at least 500 markers. 19. The method of embodiment 1, wherein the set of at least 10 markers comprises at least 1,000 markers. 20. The method of embodiment 1, wherein a significant change between said first measurement and said second measurement comprises a change of at least 10% in a marker implicated in a disorder. 21. The method of embodiment 1, or any of the above embodiments, wherein a significant change between said first measurement and said second measurement comprises a change of at least 10% in a plurality of markers implicated in a common disorder. 22. The method of embodiment 1, or any of the above embodiments, wherein a significant change between said first measurement and said second measurement comprises a change of at least 20% in a marker implicated in a disorder. 23. The method of embodiment 1, or any of the above embodiments, wherein a significant change between said first measurement and said second measurement comprises a change of at least 20% in a plurality of markers implicated in a common disorder. 24. The method of embodiment 1, or any of the above embodiments, wherein a significant change between said first measurement and said second measurement comprises a change of at least 10% in at least 5 markers implicated in a common disorder. 25. The method of embodiment 1, or any of the above embodiments, wherein a significant change between said first measurement and said second measurement comprises a determination that said first measurement and said second measurement are statistically distinguishable. 26. The method of embodiment 1, or any of the above embodiments, wherein the individual undergoes a treatment prior to the second time point. 27. A method of constructing a biomarker panel indicative of health status of a condition in an individual, comprising receiving biomarker information from a plurality of individuals, said biomarker information obtained from blood samples stored as spots on a plurality of dry solid matrixes, said biomarker information comprising at least 20 biomarkers per blood sample, and said plurality of individuals comprising individuals exhibiting the condition and individuals not exhibiting the condition; quantifying said biomarker information comprising at least 20 biomarkers per blood sample stored on a plurality of dry solid matrixes; identifying biomarkers having biomarker levels that correlate to health status of the condition; and assembling at least some of said biomarkers having biomarker levels that correlate to health status of the condition into a biomarker panel indicative of health status of a condition in an individual. 28. The method of embodiment 27, or any of the above embodiments, wherein said biomarker information comprises at least 50 biomarkers. 29. The method of embodiment 27, or any of the above embodiments, wherein said biomarker information comprises at least 100 biomarkers. 30. The method of embodiment 27, or any of the above embodiments, wherein said biomarker information comprises at least 200 biomarkers. 31. The method of embodiment 27, or any of the above embodiments, wherein said biomarker information comprises at least 500 biomarkers. 32. The method of embodiment 27, or any of the above embodiments, wherein said biomarker information comprises at least 1000 biomarkers. 33. A health status database comprising biomarker accumulation levels for at least 20 biomarkers, said biomarker accumulation levels measured at a plurality of time points, such that a change in biomarker accumulation levels over time is detectable in the database. 34. The health status database of embodiment 33, or any of the above embodiments, comprising biomarker accumulation levels for at least 50 biomarkers. 35. The health status database of embodiment 33, or any of the above embodiments, comprising biomarker accumulation levels for at least 100 biomarkers. 36. The health status database of embodiment 33, or any of the above embodiments, comprising biomarker accumulation levels for at least 200 biomarkers. 37. The health status database of embodiment 33, or any of the above embodiments, comprising biomarker accumulation levels for at least 500 biomarkers. 38. The health status database of embodiment 33, or any of the above embodiments, comprising biomarker accumulation levels for at least 1000 biomarkers. 39. The health status database of embodiment 33, or any of the above embodiments, comprising biomarker accumulation levels for at least 2000 biomarkers. 40. The health status database of embodiment 33, or any of the above embodiments, comprising biomarker accumulation levels for at least 5000 biomarkers. 41. The health status database of embodiment 33, or any of the above embodiments, comprising biomarker accumulation levels for at least 10,000 biomarkers. 42. The health status database of embodiment 33, or any of the above embodiments, wherein said plurality of time points comprises at least 3 time points. 43. The health status database of embodiment 33, or any of the above embodiments, wherein said plurality of time points comprises at least 5 time points. 44. The health status database of embodiment 33, or any of the above embodiments, wherein said plurality of time points comprises at least 10 time points. 45. The health status database of embodiment 33, or any of the above embodiments, wherein said plurality of time points comprises time points obtained over at least 1 week. 46. The health status database of embodiment 33, or any of the above embodiments, wherein said plurality of time points comprises time points obtained over at least 6 months. 47. The health status database of embodiment 33, or any of the above embodiments, wherein said plurality of time points comprises a time point subsequent to a treatment administered to an individual. 48. The health status database of embodiment 33, or any of the above embodiments, wherein said database comprises biomarker accumulation levels for biomarkers from a single individual. 49. The health status database of embodiment 33, or any of the above embodiments, wherein said database comprises biomarker accumulation levels for biomarkers from a plurality of individuals. 50. The health status database of embodiment 33, or any of the above embodiments, wherein said database comprises biomarker accumulation levels obtained from a sample fluid. 51. The health status database of embodiment 50, or any of the above embodiments, wherein said sample fluid is deposited on a dry solid matrix. 52. The health status database of embodiment 50, or any of the above embodiments, wherein said sample fluid is blood. 53. The health status database of embodiment 50, or any of the above embodiments, wherein said sample fluid is plasma. 54. A method of identifying a health status change in an individual comprising obtaining a dried fluid sample from the individual; measuring at least 15 circulating markers present in the dried fluid sample; comparing levels of the at least 15 circulating markers present in the dried fluid sample to levels of the at least 15 circulating markers stored in a database; and identifying a health status change in the individual when at least some of the at least 15 circulating markers present in the dried fluid sample differ significantly from levels of the at least 15 circulating markers stored in a database. 55. The method of embodiment 54, or any of the above embodiments, comprising identifying biomarkers present in the dried fluid sample that differ significantly from levels of the at least 15 circulating markers stored in the database, identifying a health condition related to at least some of the biomarkers present in the dried fluid sample that differ significantly from levels of the at least 15 circulating markers stored in the database; and identifying a health status change in the health condition in the individual. 56. The method of embodiment 54, or any of the above embodiments, wherein the dried fluid sample is a dried blood sample. 57. The method of embodiment 55, or any of the above embodiments, wherein the condition is colorectal health status. 58. The method of embodiment 55, or any of the above embodiments, wherein the condition is coronary artery status. 59. The method of embodiment 55, or any of the above embodiments, wherein the condition is inflammation response status. 60. The method of embodiment 55, or any of the above embodiments, wherein the condition is a cancer status. 61. The method of embodiment 55, or any of the above embodiments, comprising measuring at least 20 circulating markers. 62. The method of embodiment 55, or any of the above embodiments, comprising measuring at least 30 circulating markers. 63. The method of embodiment 55, or any of the above embodiments, comprising measuring at least 50 circulating markers. 64. The method of embodiment 55, or any of the above embodiments, comprising measuring at least 100 circulating markers. 65. The method of embodiment 55, or any of the above embodiments, comprising measuring at least 200 circulating markers. 66. The method of embodiment 55, or any of the above embodiments, comprising measuring at least 500 circulating markers. 67. The method of embodiment 55, or any of the above embodiments, comprising measuring at least 1000 circulating markers. 68. The method of embodiment 55, or any of the above embodiments, comprising measuring at least 2000 circulating markers. 69. The method of embodiment 55, or any of the above embodiments, comprising measuring at least 5,000 circulating markers. 70. The method of embodiment 55, or any of the above embodiments, wherein the circulating markers stored in a database comprise markers obtained from the individual at at least one previous time point. 71. The method of embodiment 61, or any of the above embodiments, wherein the circulating markers stored in a database comprise markers obtained from the individual at at least three previous time points. 72. The method of embodiment 61, or any of the above embodiments, wherein the circulating markers stored in a database comprise markers obtained from the individual at at least 10 previous time points. 73. The method of embodiment 61, or any of the above embodiments, wherein the circulating markers stored in a database comprise markers obtained from the individual over at least 6 months. 74. The method of embodiment 61, or any of the above embodiments, wherein the circulating markers stored in a database comprise markers obtained from the individual over at least 12 months. 75. The method of embodiment 55, or any of the above embodiments, wherein the circulating markers stored in a database comprise reference biomarkers that differ significantly from levels of the at least 1,000 circulating markers stored in the database. 76. The method of embodiment 55, or any of the above embodiments, wherein the sample is collected subsequent to administering a treatment to the individual. 77. A method of obtaining at least 20 features of biomarker information from a dried fluid sample comprising: obtaining a dried fluid spot on a solid matrix; subjecting the dried fluid spot to a digestion reaction to free dried fluid spot biomarkers from the solid matrix; resuspending the dried fluid spot biomarkers; subjecting the dried fluid spot biomarkers to mass spectrometric analysis comprising an LC gradient of no more than 15 minutes; and recovering at least 20 features from the mass spectrometric analysis. 78. The method of embodiment 77, or any of the above embodiments, wherein the fluid is blood 79. The method of embodiment 77, or any of the above embodiments, wherein the solid matrix comprises a paper backing. 80. The method of embodiment 77, or any of the above embodiments, wherein the digestion reaction comprises TFE incubation. 81. The method of embodiment 77, or any of the above embodiments, wherein the LC gradient involves no more than 10 minutes. 82. The method of embodiment 77, or any of the above embodiments, wherein the LC gradient involves no more than 7 minutes. 83. The method of embodiment 77, or any of the above embodiments, comprising obtaining at least 30 features from the mass spectrometric analysis. 84. The method of embodiment 77, or any of the above embodiments, comprising obtaining at least 50 features from the mass spectrometric analysis. 85. The method of embodiment 77, or any of the above embodiments, comprising obtaining at least 100 features from the mass spectrometric analysis. 86. The method of embodiment 77, or any of the above embodiments, comprising obtaining at least 200 features from the mass spectrometric analysis. 87. The method of embodiment 77, or any of the above embodiments, comprising obtaining at least 500 features from the mass spectrometric analysis. 88. The method of embodiment 77, or any of the above embodiments, comprising obtaining at least 1000 features from the mass spectrometric analysis. 89. The method of embodiment 77, or any of the above embodiments, comprising obtaining at least 2000 features from the mass spectrometric analysis. 90. The method of embodiment 77, or any of the above embodiments, comprising obtaining at least 5000 features from the mass spectrometric analysis. 91. The method of embodiment 77, or any of the above embodiments, comprising obtaining at least 10,000 features from the mass spectrometric analysis. 92. The method of embodiment 77, or any of the above embodiments, wherein the features of biomarker information are obtained such that there is a variability of no greater than 50% across duplicate spots on a solid matrix. 93. The method of embodiment 77, or any of the above embodiments, wherein the features of biomarker information are obtained such that there is a variability of no greater than 25% across duplicate spots on a solid matrix. 94. The method of embodiment 77, or any of the above embodiments, wherein the features of biomarker information are obtained such that there is a variability of no greater than 7% across duplicate spots on a solid matrix. 95. The method of embodiment 77, or any of the above embodiments, wherein subjecting the dried blood spot biomarkers to mass spectrometric analysis comprises introducing biomarker standards for at least some of the at least 20 features. 96. A method for using computer readable media to perform a health related assessment comprising: a. acquiring a sample from a user, or any of the above embodiments, wherein the sample comprises a dried biological fluid; b. receiving biometric data from said user, or any of the above embodiments, wherein the biometric data is provided by a tangible storage media comprising non-transitory instructions executable by a processor to cause operations to be performed; c. processing the sample and biometric data using a computer system configured for operating computer readable media, or any of the above embodiments, wherein processing the sample comprises extracting one or more features from the sample or biometric data and classifying the sample or biometric data using a trained classifier; and d. displaying the results of the classifier to said user on a user operated device comprising a screen through one or more graphical user interface(s). 97. The method of embodiment 96, or any of the above embodiments, wherein the dried biological fluid comprises a dried blood spot. 98. The acquiring step of embodiment 96, or any of the above embodiments, wherein the sample is acquired at a location separate from the point of collection for the sample. 99. The receiving step of embodiment 96, or any of the above embodiments, wherein the tangible storage media is physically coupled to the body of the user. 100. The method of embodiment 96, or any of the above embodiments, wherein the biometric data is transmitted by a tangible storage media housed in the user operated device. 101. The method of embodiment 96, or any of the above embodiments, wherein the user operated device provides a recommendation customized to the user, based on the results of the trained classifier. 102. The method of embodiment 101, or any of the above embodiments, wherein the biometric data pertains to a physical state experienced by a user, or any of the above embodiments, wherein the physical state is detected by a user operated device physically coupled to the user. 103. The method of embodiment 96, or any of the above embodiments, wherein said one or more features are extracted from the sample as well as from the biometric data. 104. A system for providing point-of-care detection of a health state in an individual, or any of the above embodiments, comprising: a. two or more user provided health data inputs, or any of the above embodiments, wherein at least one input comprises a biological sample, and at least one input comprises electronically provided feedback; b. a computer system configured for operating computer readable media; wherein processing the sample comprises extracting one or more features from the sample or biometric data, and classifying the sample or biometric data using a trained classifier; c. a user operated device configured to display the results of health data or biometric readings classified by the computer executable code; and d. an interface for displaying the data to the user. 105. The system of embodiment 104, or any of the above embodiments, wherein the user operated device comprises the tangible storage media. 106. The system of embodiment 104, or any of the above embodiments, wherein the user operated device is coupled to the user. 107. The system of embodiment 104, or any of the above embodiments, wherein the inputs comprise at least one of the following: insulin levels, alertness, health, congestion, body condition, gastrointestinal health, exercise frequency, amount of sleep, diet, hunger level, time of collection, date of collection, weather at time of collection, pollen levels at time of collection, and infectious disease frequency at time of collection. 108. The system of embodiment 104, or any of the above embodiments, wherein the outputs of the system comprise an assessment of at least one of the following: expected athletic performance, expected mental capacity, oncoming illness, expected tolerance for stress, and expected response to environmental conditions. 109. The system of embodiment 104, or any of the above embodiments, wherein the outputs of the system comprise a prediction of a health event. 110. The system of embodiment 109, or any of the above embodiments, wherein the individual is asymptomatic for the health event. 111. The system of embodiment 104, further comprising a tangible storage media comprising non-transitory instructions executable by a processor to cause operations to be performed including providing one or more electronically collected biometric readings. 112. A method for estimating a future health state of a user, using a non-transient computer readable media containing program instructions causing a computer to perform a method comprising the steps of: a. constructing a predictive model based on molecular, activity, or personal data received from a user, or any of the above embodiments, wherein the data is derived from biological samples, biometric readings, and user provided personal information; b. receiving user provided input or dataset; c. extracting multiple features from a data set, or any of the above embodiments, wherein the data set comprises data derived from a biological sample, biometric readings provided by a remote electronic device, and survey feedback provided by a user; d. processing input for one or more features to build a predictive model; e. providing tailored feedback to the user based on the resulting predictive model; f revising the predictive model based on the accuracy of the feedback provided to the user; g. repeating steps (b) and (f) so as to iteratively improve the predictive accuracy of the predictive model. 113. The method of embodiment 112, or any of the above embodiments, wherein step (a) comprises a model that has been trained on data from other individuals, but not from the user. 114. The method of embodiment 112, or any of the above embodiments, wherein step (a) comprises a model that has been trained on data from the user as well as other individuals. 115. The method of embodiment 112, or any of the above embodiments, wherein the tailored feedback comprises predictions of positive or negative health outcomes or precautions that the user can take. 116. The method of embodiment 112, or any of the above embodiments, wherein the tailored feedback is assessed with regards to key events disclosed by the user including performance related activities. 117. A method of biomarker database generation comprising identifying a biomarker set to include in a database; obtaining reference biomarker molecules that comprise biomarker components that differ in mass spectrometric migration from the protein biomarkers; obtaining at least one sample to assay for inclusion into the database; providing the reference protein biomarker molecules to the sample; subjecting the sample to mass spectrometric analysis to generate a mass spectrometric analysis output; identifying the reference biomarker molecules in the mass spectrometric analysis output; and scoring mass spectrometric spots predictably offset from the reference protein biomarker molecules as spots indicative of reference protein biomarker molecules. 118. The method of embodiment 117, or any of the above embodiments, wherein the reference biomarker molecules comprise at least one of proteins, lipids, cholesterols, steroids, drugs, and metabolites. 119. The method of embodiment 117, or any of the above embodiments, wherein the reference biomarker molecules comprise proteins. 120. The method of embodiment 117, or any of the above embodiments, wherein the reference biomarker molecules comprise at least 10 different molecules. 121. The method of embodiment 117, or any of the above embodiments, wherein the reference biomarker molecules comprise at least 20 molecules. 122. The method of embodiment 117, or any of the above embodiments, wherein the reference biomarker molecules comprise at least 30 molecules. 123. The method of embodiment 117, or any of the above embodiments, wherein the reference biomarker molecules comprise at least 40 molecules. 124. The method of embodiment 117, or any of the above embodiments, wherein the reference biomarker molecules comprise at least 50 molecules. 125. The method of embodiment 117, or any of the above embodiments, wherein the reference biomarker molecules comprise at least 100 molecules. 126. The method of embodiment 117, or any of the above embodiments, wherein the reference biomarker molecules comprise at least 200 different molecules. 127. The method of embodiment 117, or any of the above embodiments, wherein the reference biomarker molecules comprise at least 300 different molecules. 128. The method of embodiment 117, or any of the above embodiments, wherein the reference biomarker molecules comprise at least 400 different molecules. 129. The method of embodiment 117, or any of the above embodiments, wherein the reference biomarker molecules comprise at least 500 different molecules. 130. The method of embodiment 117, or any of the above embodiments, wherein the reference biomarker molecules comprise at least 1000 different molecules. 131. The method of embodiment 117, or any of the above embodiments, wherein the reference biomarker molecules are isotopically labeled. 132. The method of embodiment 117, or any of the above embodiments, wherein the reference biomarker molecules comprise molecules labeled using at least one of H2, H3, heavy nitrogen, heavy carbon, heavy oxygen, S35, P33, P32, and isotopic selenium. 133. The method of embodiment 117, or any of the above embodiments, wherein the reference biomarker molecules are chemically modified. 134. The method of embodiment 117, or any of the above embodiments, wherein the reference biomarker molecules are at least one of oxidized, acetylated, methylated, and phosphorylated. 135. The method of embodiment 117, or any of the above embodiments, wherein the reference biomarker molecules are nonhuman homologs of human proteins in the biomarker set. 136. The method of embodiment 117, or any of the above embodiments, wherein the at least one sample comprises a dried blood sample. 137. The method of embodiment 117, or any of the above embodiments, wherein the at least one sample comprises a dried plasma sample. 138. The method of embodiment 117, or any of the above embodiments, wherein the at least one sample is collected on a solid backing. 139. The method of embodiment 117, or any of the above embodiments, wherein the at least one sample comprises a collected breath sample. 140. The method of embodiment 117, or any of the above embodiments, wherein the at least one sample comprises 10 samples. 141. The method of embodiment 117, or any of the above embodiments, wherein the at least one sample comprises 20 samples. 142. The method of embodiment 117, or any of the above embodiments, wherein the at least one sample comprises 50 samples. 143. The method of embodiment 117, or any of the above embodiments, wherein the at least one sample comprises 100 samples. 144. The method of embodiment 117, or any of the above embodiments, wherein the at least one sample comprises 200 samples. 145. The method of embodiment 117, or any of the above embodiments, wherein the at least one sample comprises 500 samples. 146. The method of embodiment 117, or any of the above embodiments, wherein the at least one sample comprises 1,000 samples. 147. The method of embodiment 117, or any of the above embodiments, wherein the at least one sample comprises samples collected from an individual at different time points. 148. The method of embodiment 117, or any of the above embodiments, wherein the at least one sample comprises samples collected from an individual before and after a treatment. 149. The method of embodiment 117, or any of the above embodiments, wherein the at least one sample comprises samples collected from a plurality of individuals. 150. The method of embodiment 117, or any of the above embodiments, wherein the at least one sample comprises samples collected from individuals differing in at least one health status. 151. The method of embodiment 117, or any of the above embodiments, wherein subjecting the sample to mass spectrometric analysis comprises an LC gradient run for no more than 15 minutes. 152. The method of embodiment 117, or any of the above embodiments, wherein subjecting the sample to mass spectrometric analysis comprises an LC gradient run for no more than 10 minutes. 153. The method of embodiment 117, or any of the above embodiments, wherein subjecting the sample to mass spectrometric analysis comprises an LC gradient run for no more than 7 minutes. 154. The method of embodiment 117, or any of the above embodiments, wherein subjecting the sample to mass spectrometric analysis comprises an LC gradient run for no more than 1 minute. 155. The method of embodiment 117, or any of the above embodiments, wherein subjecting the sample to mass spectrometric analysis comprises enzymatic digestion of the sample. 156. The method of embodiment 117, or any of the above embodiments, wherein subjecting the sample to mass spectrometric analysis comprises TFE incubation. 157. The method of embodiment 117, or any of the above embodiments, wherein identifying the reference protein biomarker molecules in the mass spectrometric analysis output is computer-automated. 158. The method of embodiment 117, or any of the above embodiments, wherein identifying the reference protein biomarker molecules in the mass spectrometric analysis output does not comprise confirmation by a user. 159. The method of embodiment 117, or any of the above embodiments, wherein scoring mass spectrometric spots predictably offset from the reference protein biomarker molecules as spots indicative of reference protein biomarker molecules is computer-automated. 160. The method of embodiment 117, or any of the above embodiments, wherein scoring mass spectrometric spots predictably offset from the reference protein biomarker molecules as spots indicative of reference protein biomarker molecules is confirmed via ms2 mass spectrometric analysis. 161. The method of embodiment 117, or any of the above embodiments, wherein scoring mass spectrometric spots predictably offset from the reference protein biomarker molecules as spots indicative of reference protein biomarker molecules does not comprise confirmation by a user. 162. The method of embodiment 117, or any of the above embodiments, comprising quantifying native biomarker spot amounts. 163. The method of embodiment 117, or any of the above embodiments, comprising determining native biomarker spot signal intensity relative to reference protein biomarker molecule spot intensity. 164. The method of embodiment 117, or any of the above embodiments, comprising entering results of the scoring into a database comprising at least 100 sample results. 165. The method of embodiment 117, or any of the above embodiments, comprising entering results of the scoring into a database comprising at least 1,000 sample results. 166. The method of embodiment 117, or any of the above embodiments, comprising entering results of the scoring into a database comprising at least 10,000 sample results. 167. The method of embodiment 117, or any of the above embodiments, comprising entering results of the scoring into a database comprising at least 100,000 sample results. 168. The method of embodiment 117, or any of the above embodiments, comprising entering results of the scoring into a database comprising at least 1,000,000 sample results. 169. The method of embodiment 117, or any of the above embodiments, comprising entering results of the scoring into a database comprising at least 1,000,000,000 sample results. 170. A composition comprising a dried blood extract to which a plurality of populations of mass labeled proteins are added. 171. The composition of embodiment 170, or any of the above embodiments, wherein the plurality of populations of mass labeled proteins comprise isotope labeled proteins. 172. The composition of embodiment 170, or any of the above embodiments, wherein the plurality of populations of mass labeled proteins comprise chemically labeled proteins. 173. The composition of embodiment 170, or any of the above embodiments, wherein the plurality of populations of mass labeled proteins comprise nonhuman homologs of human proteins. 174. The composition of embodiment 170, or any of the above embodiments, wherein the plurality of populations of isotope labeled proteins comprises at least 3 populations. 175. The composition of embodiment 170, or any of the above embodiments, wherein the plurality of populations of isotope labeled proteins comprises at least 4 populations. 176. The composition of embodiment 170, or any of the above embodiments, wherein the plurality of populations of isotope labeled proteins comprises at least 5 populations. 177. The composition of embodiment 170, or any of the above embodiments, wherein the plurality of populations of isotope labeled proteins comprises at least 10 populations. 178. The composition of embodiment 170, or any of the above embodiments, wherein the plurality of populations of isotope labeled proteins comprises at least 15 populations. 179. The composition of embodiment 170, or any of the above embodiments, wherein the plurality of populations of isotope labeled proteins comprises at least 20 populations. 180. The composition of embodiment 170, or any of the above embodiments, wherein the plurality of populations of isotope labeled proteins comprises at least 50 populations. 181. The composition of embodiment 170, or any of the above embodiments, wherein the plurality of populations of isotope labeled proteins comprises at least 100 populations. 182. The composition of embodiment 170, or any of the above embodiments, wherein the plurality of populations of isotope labeled proteins comprises at least 1000 populations. 183. The composition of embodiment 170, or any of the above embodiments, wherein the plurality of populations of isotope labeled proteins comprises proteins indicative of a health status when detected in circulating blood. 184. The composition of embodiment 170, or any of the above embodiments, wherein the plurality of populations of labeled proteins comprises proteins labeled using at least one of H2, H3, heavy nitrogen, heavy carbon, heavy oxygen, S35, P33, P32, and isotopic selenium. 185. The composition of embodiment 170, or any of the above embodiments, wherein the plurality of populations of labeled proteins comprises proteins labeled using at least one of oxidation, acetylation, methylation, and phosphorylation. 186. The composition of embodiment 170, or any of the above embodiments, wherein the dried blood extract is subject to enzymatic digestion. 187. The composition of embodiment 170, or any of the above embodiments, wherein the dried blood extract is subject to TFE/Trypsin digestion. 188. The composition of embodiment 170, or any of the above embodiments, wherein the composition is configured on a mass spectrometric output display. 189. The composition of embodiment 170, or any of the above embodiments, wherein the dried blood extract is volatilized. 190. A computer-implemented method of generating a diagnostic tool for health categorization of a human subject by applying machine learning to a diagnostic instrument for health categorization, or any of the above embodiments, wherein the diagnostic instrument comprises inputs for a set of biomarkers, the computer implemented method comprising: on a computer system having at least one processor and a memory storing at least one computer program for execution by the at least one processor, the at least one program having instructions for: receiving as input biomarker information comprising mass spectrometric data measured from a dried liquid taken from at least one individual of known health status relevant to the health categorization; analyzing the diagnostic outcomes and the biomarker information comprising mass spectrometric data measured from the dried liquid sample taken from at least one individual of known health status relevant to the health categorization, to distinguish a panel of biomarkers informative as to health categorization; determining the accuracy of the panel of biomarkers by testing the biomarker panel against an independent source of biomarker data; generating the diagnostic tool for health categorization of a human subject, wherein the diagnostic tool comprises the panel of biomarkers; and configuring a computer device accessible by a user to receive levels of the panel of biomarkers, and to provide the user a health categorization of the human subject. 191. The computer-implemented method of embodiment 190, or any of the above embodiments, wherein the dried liquid sample is a dried blood sample. 192. The computer-implemented method of embodiment 190, or any of the above embodiments, wherein the dried liquid sample is a dried plasma sample. 193. The computer-implemented method of embodiment 190, or any of the above embodiments, wherein the set of biomarkers comprises at least 20 mass spectrometric data points. 194. The computer-implemented method of embodiment 191, or any of the above embodiments, wherein the at least 20 mass spectrometric data points exhibit a median CV of no greater than 50% when mass spectrometric data is regathered from a common dried blood sample on a common collection device. 195. The computer-implemented method of embodiment 191, or any of the above embodiments, wherein the at least 20 mass spectrometric data points exhibit a median CV of no greater than 25% when mass spectrometric data is regathered from a common dried blood sample on a common collection device. 196. The computer-implemented method of embodiment 191, or any of the above embodiments, wherein the at least 20 mass spectrometric data points exhibit a median CV of no greater than 7% when mass spectrometric data is regathered from a common dried blood sample on a common collection device. 197. The computer-implemented method of embodiment 190, or any of the above embodiments, wherein the at least 20 mass spectrometric data points exhibit a median CV of no greater than 50% when mass spectrometric data is regathered from a plurality of dried blood samples taken from distinct collection devices. 198. The computer-implemented method of embodiment 190, or any of the above embodiments, wherein the at least 20 mass spectrometric data points exhibit a median CV of no greater than 37% when mass spectrometric data is regathered from a plurality of dried blood samples taken from distinct collection devices. 199. The computer-implemented method of embodiment 190, or any of the above embodiments, wherein the at least 20 mass spectrometric data points exhibit a median CV of no greater than 26% when mass spectrometric data is regathered from a plurality of dried blood samples taken from distinct collection devices. 200. The computer-implemented method of embodiment 190, or any of the above embodiments, wherein the set of biomarkers comprises at least one of age information, gender information, sleep information, geographical information relating to a point of sample collection, temporal information relating to a time of sample collection, and behavioral information relating to human subject alertness. 201. The computer-implemented method of embodiment 190, or any of the above embodiments, wherein the set of biomarkers comprises at least 5 biomarkers. 202. The computer-implemented method of embodiment 190, or any of the above embodiments, wherein the set of biomarkers comprises at least 6 biomarkers. 203. The computer-implemented method of embodiment 190, or any of the above embodiments, wherein the set of biomarkers comprises at least 7 biomarkers. 204. The computer-implemented method of embodiment 190, or any of the above embodiments, wherein the set of biomarkers comprises at least 8 biomarkers. 205. The computer-implemented method of embodiment 190, or any of the above embodiments, wherein the set of biomarkers comprises at least 9 biomarkers. 206. The computer-implemented method of embodiment 190, or any of the above embodiments, wherein the set of biomarkers comprises at least 10 biomarkers. 207. The computer-implemented method of embodiment 190, or any of the above embodiments, wherein the set of biomarkers comprises at least 15 biomarkers. 208. The computer-implemented method of embodiment 190, or any of the above embodiments, wherein the machine learning comprises a technique chosen from the group consisting of ADTree, BFTree, ConjunctiveRule, DecisionStump, Filtered Classifier, J48, J48Graft, JRip, LADTree, NNge, OneR, OrdinalClassClassifier, PART, Ridor, SimpleCart, Random Forest and SVM. 209. The computer-implemented method of embodiment 190, or any of the above embodiments, wherein the independent source comprises biomarker information obtained from a plurality of individuals of known health categorization. 210. The computer-implemented method of embodiment 190, or any of the above embodiments, wherein the independent source comprises biomarker information obtained from the human subject at a plurality of time points. 211. The computer-implemented method of embodiment 190, or any of the above embodiments, wherein the independent source comprises biomarker information obtained from the human subject prior to administering a treatment to the subject. 212. The computer-implemented method of embodiment 190, or any of the above embodiments, wherein the categorization is a cancer categorization. 213. The computer-implemented method of embodiment 190, or any of the above embodiments, wherein the categorization is at least one of a colorectal, skin, lung, throat, blood, brain, breast, and prostate cancer categorization. 214. The computer-implemented method of embodiment 190, or any of the above embodiments, wherein the categorization is an effective age categorization. 215. The computer-implemented method of embodiment 190, or any of the above embodiments, wherein the categorization is a gender categorization. 216. The computer-implemented method of embodiment 190, or any of the above embodiments, wherein the categorization is a demographic categorization. 217. The computer-implemented method of embodiment 190, or any of the above embodiments, wherein the dried blood sample is stored on a planar collection matrix. 218. The computer-implemented method of embodiment 190, or any of the above embodiments, wherein the dried blood sample is stored in a porous collection volume. 219. A processor comprising a memory unit configured to receive biomarker information comprising mass spectrometric data generated from at least one dried sample from a human subject; a reference unit comprising a reference dataset comprising biomarker information comprising mass spectrometric data generated from at least one dried sample of at least one individual; a processor unit configured to categorize the sample relative to the reference dataset; and an output unit configured to indicate the categorization of the sample relative to the reference dataset. 220. The processor of embodiment 219, or any of the above embodiments, wherein the dried sample is at least one of a blood sample, a urine sample, a sweat sample, a tear sample and a saliva sample. 221. The processor of embodiment 219, or any of the above embodiments, wherein the dried sample is a dried blood sample. 222. The processor of embodiment 219, or any of the above embodiments, wherein the memory unit is configured to receive dried blood sample biomarker information. 223. The processor of embodiment 219, or any of the above embodiments, wherein the memory unit is configured to receive biomarker information comprising at least 20 mass spectrometric data points obtained per dried blood sample. 224. The processor of embodiment 219, or any of the above embodiments, wherein the memory unit is configured to receive biomarker information comprising at least 30 mass spectrometric data points obtained per dried blood sample. 225. The processor of embodiment 219, or any of the above embodiments, wherein the memory unit is configured to receive biomarker information comprising at least 50 mass spectrometric data points obtained per dried blood sample. 226. The processor of embodiment 219, or any of the above embodiments, wherein the memory unit is configured to receive biomarker information comprising at least 100 mass spectrometric data points obtained per dried blood sample. 227. The processor of embodiment 219, or any of the above embodiments, wherein the memory unit is configured to receive biomarker information comprising at least 200 mass spectrometric data points obtained per dried blood sample. 228. The processor of embodiment 219, or any of the above embodiments, wherein the memory unit is configured to receive biomarker information comprising at least 500 mass spectrometric data points obtained per dried blood sample. 229. The processor of embodiment 219, or any of the above embodiments, wherein the memory unit is configured to receive biomarker information comprising at least 1,000 mass spectrometric data points obtained per dried blood sample. 230. The processor of embodiment 223, or any of the above embodiments, wherein the at least 20 mass spectrometric data points exhibit a median CV of no greater than 50% when mass spectrometric data is regathered from a common dried blood sample. 231. The processor of embodiment 223, or any of the above embodiments, wherein the at least 20 mass spectrometric data points exhibit a median CV of no greater than 25% when mass spectrometric data is regathered from a common dried blood sample. 232. The processor of embodiment 223, or any of the above embodiments, wherein the at least 20 mass spectrometric data points exhibit a median CV of no greater than 7% when mass spectrometric data is regathered from a common dried blood sample. 233. The processor of embodiment 223, or any of the above embodiments, wherein the at least 20 mass spectrometric data points exhibit a median CV of no greater than 50% when mass spectrometric data is regathered from a plurality of dried blood sample. 234. The processor of embodiment 223, or any of the above embodiments, wherein the at least 20 mass spectrometric data points exhibit a median CV of no greater than 37% when mass spectrometric data is regathered from a plurality of dried blood sample. 235. The processor of embodiment 223, or any of the above embodiments, wherein the at least 20 mass spectrometric data points exhibit a median CV of no greater than 26% when mass spectrometric data is regathered from a plurality of dried blood sample. 236. The processor of embodiment 219, or any of the above embodiments, wherein the memory unit is configured to receive at least one of age information, gender information, sleep information, geographical information relating to a point of sample collection, temporal information relating to a time of sample collection, and behavioral information relating to human subject alertness. 237. The processor of embodiment 219, or any of the above embodiments, wherein the reference unit comprising a reference dataset comprising biomarker information is obtained from at least one individual of known health status such that the reference dataset is indicative of a health status categorization. 238. The processor of embodiment 219, or any of the above embodiments, wherein the reference unit comprising a reference dataset comprising biomarker information is obtained from a population of individuals characterized as to health status. 239. The processor of embodiment 219, or any of the above embodiments, wherein the reference unit comprising a reference dataset comprising biomarker information comprises expected biomarker levels informed by a health model. 240. The processor of embodiment 219, or any of the above embodiments, wherein the processor unit configured to categorize the sample relative to the reference dataset assigns the sample a percentile value relative to the reference dataset. 241. A biomarker analysis display comprising at least 20 mass spectrometric data points obtained from a single dried sample; and at least 3 mass-shifted labeled peptide spots each indicative of an expected location of a nearby mass spectrometric data point. 242. The biomarker analysis display of embodiment 241, or any of the above embodiments, wherein the at least 3 mass-shifted labeled peptide spots each correspond to at least one known biomarker. 243. The biomarker analysis display of embodiment 241, or any of the above embodiments, wherein the at least 3 mass-shifted labeled peptide spots each correspond to at least one known biomarker. 244. The biomarker analysis display of embodiment 241, or any of the above embodiments, wherein the at least 3 mass-shifted labeled peptide spots each correspond to at least one FDA recognized quantitative biomarkers. 245. The biomarker analysis display of embodiment 241, or any of the above embodiments, wherein the at least 3 mass-shifted labeled peptide spots each correspond to a constituent of a panel informative of a health categorization. 246. The biomarker analysis display of embodiment 241, or any of the above embodiments, comprising at least 10 mass shifted peptide spots each correspond to a constituent of a panel informative of a health categorization. 247. The biomarker analysis display of embodiment 245, or any of the above embodiments, wherein the health categorization is at least one of a cancer categorization, an age categorization, a demographic categorization, a fitness categorization, a communicable disease categorization, and a non-communicable disease categorization. 248. The biomarker analysis display of embodiment 241, or any of the above embodiments, wherein the at least 20 mass spectrometric data points exhibit a median CV of no greater than 50% when mass spectrometric data is regathered from a common dried blood sample. 249. The biomarker analysis display of embodiment 241, or any of the above embodiments, wherein the at least 20 mass spectrometric data points exhibit a median CV of no greater than 25% when mass spectrometric data is regathered from a common dried blood sample. 250. The biomarker analysis display of embodiment 241, or any of the above embodiments, wherein the at least 20 mass spectrometric data points exhibit a median CV of no greater than 7% when mass spectrometric data is regathered from a common dried blood sample. 251. The biomarker analysis display of embodiment 241, or any of the above embodiments, wherein the at least 20 mass spectrometric data points exhibit a median CV of no greater than 50% when mass spectrometric data is regathered from a plurality of dried blood samples. 252. The biomarker analysis display of embodiment 241, or any of the above embodiments, wherein the at least 20 mass spectrometric data points exhibit a median CV of no greater than 37% when mass spectrometric data is regathered from a plurality of dried blood samples. 253. The biomarker analysis display of embodiment 241, or any of the above embodiments, wherein the at least 20 mass spectrometric data points exhibit a median CV of no greater than 26% when mass spectrometric data is regathered from a plurality of dried blood samples. 254. The biomarker analysis display of embodiment 241, or any of the above embodiments, comprising mass spectrometric data points obtained from breath exudate. 255. A biomarker analysis display comprising at least 100 mass spectrometric data points obtained from a single dried blood sample, or any of the above embodiments, wherein the at least 100 mass spectrometric data points exhibit a median CV of no greater than 7% when mass spectrometric data is regathered from a common dried blood sample. 256. A biomarker analysis display comprising at least 100 mass spectrometric data points obtained from a single dried blood sample, or any of the above embodiments, wherein the at least 100 mass spectrometric data points exhibit a median CV of no greater than 26% when mass spectrometric data is regathered from a plurality of dried blood samples. 257. A processor comprising a memory unit configured to store data indicative of health status categorization in a comparison sample, said memory unit comprising: storage capacity configured to receive reference mass spectrometry data for at least 20 mass spectrometric values derived from each of a plurality of analyzed dried samples; storage capacity to correlate said at least 20 mass spectrometric signals derived from each of a plurality of analyzed dried blood samples to non-mass spectrometry data comprising at least one of age of sample source individual, time of collection, geographical region of collection, demographic information, blood glucose levels at time of collection, sleep history at time of collection, and mental alertness at time of collection; a comparison unit configured to receive at least one individual dataset comprising mass spectrometry data for at least 50 mass spectrometric signals derived from each of a plurality of analyzed dried blood samples and non-mass spectrometry data comprising at least one of age of sample source individual, time of collection, geographical region of collection, demographic information, blood glucose levels at time of collection, sleep history at time of collection, and mental alertness at time of collection; and compare said individual dataset to said reference mass spectrometry data, such that an assessment as to whether the individual data set differs significantly from the reference dataset is made. 258. The processor of embodiment 257, or any of the above embodiments, wherein the reference dataset comprises data from samples derived from at least one individual having a known health status categorization when a sample is obtained. 259. The processor of embodiment 257, or any of the above embodiments, wherein the reference dataset and the individual dataset are derived from a common individual. 260. The processor of embodiment 257, or any of the above embodiments, wherein the reference dataset is derived from a plurality of individuals. 261. The processor of embodiment 257, or any of the above embodiments, wherein an individual dataset differing significantly from the reference dataset indicates that an individual source of the individual dataset does not share a health categorization of the reference dataset. 262. The processor of embodiment 257, or any of the above embodiments, wherein an individual dataset not differing significantly from the reference dataset indicates that an individual source of the individual dataset does share a health categorization of the reference dataset. 263. The processor of embodiment 257, or any of the above embodiments, wherein an individual dataset is assigned a percentile value relative to the reference dataset. 264. A device for dried fluid sample collection comprising a region configured to receive a sample such that the sample dries on the region, and at least three standard markers deposited on the device such that processing of the sample introduces the marker into the sample. 265. The device of embodiment 264, or any of the above embodiments, wherein the region configured to receive the sample comprises a surface having a planar face. 266. The device of embodiment 264, or any of the above embodiments, wherein the region configured to receive the sample comprises a three-dimensional volume. 267. The device of embodiment 264, or any of the above embodiments, wherein the sample comprises a body fluid. 268. The device of embodiment 264, or any of the above embodiments, wherein the sample comprises at least one of blood, saliva, urine and sweat. 269. The device of embodiment 264, or any of the above embodiments, wherein the standard marker comprises a constituent that is visualizable on a mass spectrometric output. 270. The device of embodiment 264, or any of the above embodiments, wherein the standard marker comprises a constituent that has a mass that differs from a sample constituent mass by a known amount. 271. The device of embodiment 264, or any of the above embodiments, wherein the standard marker comprises a constituent that has a mass that differs from a sample constituent mass by an amount that is readily visualizable on a mass spectrometric output. 272. The device of embodiment 264, or any of the above embodiments, wherein the standard marker comprises a constituent that has a mass that differs from a sample constituent mass by an amount comparable to a difference between an atom and a heavy isotope of that atom. 273. The device of embodiment 264, or any of the above embodiments, wherein the standard marker comprises a polypeptide. 274. The device of embodiment 264, or any of the above embodiments, wherein the standard marker comprises a lipid. 275. The device of embodiment 264, or any of the above embodiments, wherein the standard marker comprises small molecule metabolite. 276. The device of embodiment 264, or any of the above embodiments, wherein two samples extracted from a common collection device exhibit a CV of no greater than 6.5%. 277. The device of embodiment 264, or any of the above embodiments, wherein two samples from a common source extracted from distinct collection devices exhibit a CV of no greater than 25%. 278. The device of embodiment 264, or any of the above embodiments, wherein the at least three standard markers are isotopically labeled. 279. The device of embodiment 264, or any of the above embodiments, wherein the at least three standard markers are labeled using at least one of H2, H3, heavy nitrogen, heavy carbon, heavy oxygen, S35, P33, P32, and isotopic selenium. 280. The device of embodiment 264, or any of the above embodiments, wherein the at least three standard markers are chemically modified. 281. The device of embodiment 264, or any of the above embodiments, wherein the at least three standard markers are chemically labeled. 282. The device of embodiment 264, or any of the above embodiments, wherein the at least three standard markers are at least one of oxidized, acetylated, methylated, and phosphorylated. 283. The method of embodiment 117, or any of the above embodiments, wherein the at least three standard markers are nonhuman homologs of human proteins in the biomarker set. 284. A method of identifying a biomarker signal in an existing biomarker panel selected to characterize a health condition, comprising obtaining biomarker panel level information for a plurality of samples having a known health condition status, entering the biomarker panel information to a computer configured to receive the biomarker panel information applying a machine learning algorithm to the biomarker panel information to identify a subset of markers in the biomarker panel that repeatably correlate with health condition status, thereby generating a smaller panel having a comparable health characteristic status predictive power. 285. The method of embodiment 284, or any of the above embodiments, wherein obtaining biomarker panel level information comprises collecting dried samples from at least one source having a known health condition status. 286. The method of embodiment 285, or any of the above embodiments, wherein the dried samples are blood samples. 287. The method of embodiment 284, or any of the above embodiments, wherein obtaining biomarker panel level information comprises introducing standard markers into the plurality of samples. 288. The method of embodiment 287, or any of the above embodiments, wherein the standard markers comprise heavy-labeled equivalents of panel constituents. 289. The method of embodiment 284, or any of the above embodiments, comprising assessing non-panel mass spectrometric information to identify at least one additional marker to use with the subset of markers. 290. The method of embodiment 287, or any of the above embodiments, wherein the biomarker panel level information comprises mass spectrometric data. 291. The method of embodiment 287, or any of the above embodiments, wherein the biomarker panel level information comprises proteomic data. 292. The method of embodiment 287, or any of the above embodiments, wherein the biomarker panel level information comprises lipid data. 293. The method of embodiment 287, or any of the above embodiments, wherein the biomarker panel level information comprises metabolite data. 294. The method of embodiment 287, or any of the above embodiments, wherein the biomarker panel level information comprises nucleic acid data. 295. The method of embodiment 284, or any of the above embodiments, comprising obtaining accompanying data for the biomarker panel level information. 296. The method of embodiment 295, or any of the above embodiments, wherein the accompanying data comprises at least one of protein level information, RNA level information, glucose level information, sleep status information, individual alertness information, age information, gender information, demographic information, time of collection information, diet information, individual height, individual weight, individual blood pressure, environmental information at collection such as temperature, pollen status, and demographic infection status, and health condition-independent health status information such as pulmonary status information. 297. The method of embodiment 284, or any of the above embodiments, wherein the panel comprises at least 6 markers. 298. The method of embodiment 284, or any of the above embodiments, wherein the panel comprises at least 7 markers. 299. The method of embodiment 284, or any of the above embodiments, wherein the panel comprises at least 8 markers. 300. The method of embodiment 284, or any of the above embodiments, wherein the panel comprises at least 10 markers. 301. The method of embodiment 284, or any of the above embodiments, wherein the panel comprises at least 15 markers. 302. The method of embodiment 284, or any of the above embodiments, wherein the panel comprises at least 20 markers. 303. The method of embodiment 284, or any of the above embodiments, wherein the panel comprises at least 30 markers. 304. The method of embodiment 284, or any of the above embodiments, wherein the panel comprises at least 100 markers. 305. The method of embodiment 284, or any of the above embodiments, wherein the panel comprises no more than 10 markers. 306. The method of embodiment 284, or any of the above embodiments, wherein the panel comprises no more than 15 markers. 307. The method of embodiment 284, or any of the above embodiments, wherein the panel comprises no more than 20 markers. 308. The method of embodiment 284, or any of the above embodiments, wherein the panel comprises no more than 30 markers. 309. The method of embodiment 284, or any of the above embodiments, wherein the panel comprises no more than 100 markers. 310. A method of neonatal health monitoring comprising obtaining a dried fluid sample from an infant, analyzing at least 20 biomarkers from the dried fluid sample, and identifying a biomarker level characteristic of a neonatal disorder. 311. The method of embodiment 310, or any of the above embodiments, wherein the dried fluid sample comprises dried blood. 312. The method of embodiment 310, or any of the above embodiments, wherein analyzing at least 20 biomarkers from the dried fluid sample comprises introducing standard markers into the plurality of samples. 313. The method of embodiment 310, or any of the above embodiments, wherein the at least 20 biomarkers comprise proteins and metabolites. 314. The method of embodiment 312, or any of the above embodiments, wherein the standard markers comprise heavy-labeled equivalents of panel constituents. 315. A method of biomarker data accumulation comprising obtaining a dried fluid sample from at least one subject, volatilizing the dried fluid sample, subjecting the sample to mass spectrometric analysis, and identifying at least 20 biomarkers in the mass spectrometric analysis. 316. The method of embodiment 315, or any of the above embodiments, wherein the dried fluid sample comprises at least one of blood, saliva, sweat, tears and urine. 317. The method of embodiment 315, or any of the above embodiments, wherein the dried fluid sample is a blood sample. 318. The method of embodiment 315, or any of the above embodiments, wherein the dried fluid sample is a plasma sample. 319. The method of embodiment 315, or any of the above embodiments, comprising contacting the sample to at least one reference marker prior to mass spectrometric visualization. 320. The method of embodiment 319, or any of the above embodiments, wherein contacting the sample to at least one reference marker comprises depositing the at least one reference marker on a solid surface prior to contacting the sample to the solid surface. 321. The method of embodiment 319, or any of the above embodiments, wherein contacting the sample to at least one reference marker comprises adding the reference marker to the sample subsequent to resolubilizing the sample. 322. The method of embodiment 319, or any of the above embodiments, wherein contacting the sample to at least one reference marker comprises adding the reference marker to the sample subsequent to digesting the sample for mass spectrometric analysis. 323. The method of embodiment 319, or any of the above embodiments, wherein the at least one reference marker comprises a panel of reference markers that facilitates automated identification of corresponding panel constituents in the sample. 324. The method of embodiment 315, or any of the above embodiments, comprising identifying at least one reference marker-associated biomarker and at least one biomarker not associated with a reference marker. 325. The method of embodiment 315, or any of the above embodiments, comprising identifying at least 50 biomarkers in the mass spectrometric analysis. 326. The method of embodiment 315, or any of the above embodiments, comprising identifying at least 100 biomarkers in the mass spectrometric analysis. 327. The method of embodiment 315, or any of the above embodiments, comprising identifying at least 200 biomarkers in the mass spectrometric analysis. 328. The method of embodiment 315, or any of the above embodiments, comprising identifying at least 500 biomarkers in the mass spectrometric analysis. 329. The method of embodiment 315, or any of the above embodiments, comprising identifying at least 1000 biomarkers in the mass spectrometric analysis. 330. The method of embodiment 315, or any of the above embodiments, comprising identifying at least 2000 biomarkers in the mass spectrometric analysis. 331. The method of embodiment 315, or any of the above embodiments, comprising identifying at least 5000 biomarkers in the mass spectrometric analysis. 332. The method of embodiment 315, or any of the above embodiments, comprising identifying at least 10,000 biomarkers in the mass spectrometric analysis. 333. The method of embodiment 315, or any of the above embodiments, comprising obtaining a dried fluid sample from at least 10 subjects. 334. The method of embodiment 315, or any of the above embodiments, comprising obtaining a dried fluid sample from at least 20 subjects. 335. The method of embodiment 315, or any of the above embodiments, comprising obtaining a dried fluid sample from at least 50 subjects. 336. The method of embodiment 315, or any of the above embodiments, comprising obtaining a dried fluid sample from at least 100 subjects. 337. The method of embodiment 315, or any of the above embodiments, comprising obtaining a dried fluid sample from at least 200 subjects. 338. The method of embodiment 315, or any of the above embodiments, comprising obtaining a dried fluid sample from at least 500 subjects. 339. The method of embodiment 315, or any of the above embodiments, comprising obtaining a dried fluid sample from at least 1000 subjects. 340. The method of embodiment 315, or any of the above embodiments, comprising obtaining a dried fluid sample from at least 2000 subjects. 341. The method of embodiment 315, or any of the above embodiments, comprising obtaining a dried fluid sample from at least one subject at at least 2 time points. 342. The method of embodiment 341, or any of the above embodiments, wherein a treatment is administered between the at least two time points. 343. The method of embodiment 341, or any of the above embodiments, wherein a treatment is administered before at least one of the at least two time points. 344. The method of embodiment 315, or any of the above embodiments, comprising obtaining a dried fluid sample from at least one subject at at least 5 time points. 345. The method of embodiment 315, or any of the above embodiments, comprising obtaining a dried fluid sample from at least one subject at at least 10 time points. 346. The method of embodiment 315, or any of the above embodiments, comprising obtaining a dried fluid sample from at least one subject at at least 20 time points. 347. The method of embodiment 315, or any of the above embodiments, comprising obtaining a dried fluid sample from at least one subject at at least 50 time points. 348. The method of embodiment 315, or any of the above embodiments, wherein the biomarker data comprises at least one category of information selected from a list comprising protein information, nucleic acid sequence information, nucleic acid level information, glucose information, subject body temperature, subject sleep status, subject alertness, subject diet, subject age, subject gender, subject weight, subject height, subject body mass index, subject blood pressure, subject pulse rate, time of day during collection, time of year during collection, environmental conditions during collection, pollen count during collection, environmental temperature or weather during collection, contagious disease demographic status during collection, and subject respiratory status during collection. 349. A computer system comprising a memory unit configured to receive data generated through the method of any one of embodiments 315-348, and a processing unit having instructions to assess said data. 350. The computer system of embodiment 349, or any of the above embodiments, wherein said processing unit having instructions to assess said data comprises machine learning instructions. 351. The computer system of embodiment 350, or any of the above embodiments, wherein said machine learning instructions comprise feature identification instructions. 352. The computer system of embodiment 350, or any of the above embodiments, wherein said feature identification instructions comprise at least one of at least one of elastic net, information gain, and random forest imputing. 353. The computer system of embodiment 349, or any of the above embodiments, wherein said machine learning instructions comprise classifier generation instructions. 354. The computer system of embodiment 353, or any of the above embodiments, wherein the classifier instructions comprise at least one of logistic regression, SVM, random forest, and KNN classifier instructions. 355. The computer system of embodiment 349, comprising an output unit configured to display assessment results. 356. The computer system of embodiment 355, or any of the above embodiments, wherein said assessment results comprise AUC information related to a panel identified through said processing unit. 357. The computer system of embodiment 349, or any of the above embodiments, wherein said processing unit having instructions to assess said data comprises a comparison algorithm. 358. The computer system of embodiment 357, or any of the above embodiments, wherein said comparison algorithm scores said data relative to a reference panel. 359. A method of predicting onset of a symptom of a genetically inherited disorder comprising identifying an individual having a genetically inherited disorder; obtaining at least one dried fluid sample from the individual; and assaying biomarker levels in the dried fluid sample via mass spectrometric analysis. 360. The method of embodiment 359, or any of the above embodiments, wherein the individual does not exhibit a symptom of the genetically inherited disorder. 361. The method of embodiment 359, or any of the above embodiments, wherein the disorder is a cancer. 362. The method of embodiment 359, or any of the above embodiments, wherein the dried fluid sample comprises at least one of blood, saliva, urine and sweat. 363. The method of embodiment 359, or any of the above embodiments, wherein the dried fluid sample is a dried blood sample. 364. The method of embodiment 359, or any of the above embodiments, wherein assaying biomarker levels in the dried fluid sample via mass spectrometric analysis comprises performing an untargeted mass spectrometric shotgun analysis. 365. The method of embodiment 359, or any of the above embodiments, wherein assaying biomarker levels in the dried fluid sample via mass spectrometric analysis comprises determining levels of particular biomarkers. 366. The method of embodiment 365, or any of the above embodiments, wherein determining levels of particular biomarkers comprises contacting the sample to at least one reference marker. 367. The method of embodiment 366, or any of the above embodiments, wherein the at least one reference marker is a heavy-labeled analogue of at least one said particular biomarker. 368. The method of embodiment 366, or any of the above embodiments, wherein the at least one reference marker is added to a collection device prior to contacting the collection device to a sample. 369. The method of embodiment 366, or any of the above embodiments, wherein the at least one reference marker is added to a dried sample on a collection device. 370. The method of embodiment 366, or any of the above embodiments, wherein the at least one reference marker is added to a resolubilized sample. 371. The method of embodiment 366, or any of the above embodiments, wherein the at least one reference marker is added to a sample prior to enzymatic digestion. 372. The method of embodiment 366, or any of the above embodiments, wherein the at least one reference marker is added to a sample prior to mass spectrometric visualization. 373. The method of embodiment 365, or any of the above embodiments, wherein assaying biomarker levels in the dried fluid sample via mass spectrometric analysis comprises determining levels of particular biomarkers and comprises performing an untargeted mass spectrometric shotgun analysis. 374. The method of embodiment 359, or any of the above embodiments, wherein assaying biomarker levels in the dried fluid sample via mass spectrometric analysis comprises subjecting mass spectrometric data to a machine learning algorithm. 375. The method of embodiment 359, or any of the above embodiments, comprising obtaining at least two dried fluid samples from the individual at two distinct time points. 376. The method of embodiment 359, or any of the above embodiments, comprising obtaining at least five dried fluid samples from the individual at five distinct time points. 377. The method of embodiment 359, or any of the above embodiments, comprising initiating a treatment regimen when said assaying indicates a biomarker profile indicative of onset of a symptom associated with the genetically inherited disorder. 378. The method of embodiment 359, or any of the above embodiments, wherein said biomarker levels comprise levels of proteins implicated in a disease mechanism for the genetically inherited disease. 379. The method of embodiment 359, or any of the above embodiments, wherein said biomarker levels comprise levels of metabolites implicated in a disease mechanism for the genetically inherited disease 380. The method of embodiment 359, or any of the above embodiments, wherein said biomarker levels comprise levels of nucleic acids implicated in a disease mechanism for the genetically inherited disease 381. The method of embodiment 359, or any of the above embodiments, wherein said biomarker levels comprise levels of lipids implicated in a disease mechanism for the genetically inherited disease 382. The method of embodiment 359, or any of the above embodiments, wherein said biomarker levels comprise levels of cholesterols implicated in a disease mechanism for the genetically inherited disease. 383. The method of embodiment 359, or any of the above embodiments, comprising treating the individual to alleviate a symptom of the genetically inherited disorder. 384. The method of embodiment 383, or any of the above embodiments, comprising treating the individual prior to onset of the symptom. 385. The method of embodiment 383, comprising continuing sample collection and analysis to assess treatment efficacy. 386. A method comprising obtaining a dried blood spot sample; volatilizing the dried blood spot sample; subjecting the volatilized sample to mass spectrometric analysis; and displaying at least 20 mass features from the solubilized sample. 387. The method of embodiment 386, or any of the above embodiments, comprising displaying at least 1 mass-shifted reference marker added to the sample, or any of the above embodiments, wherein the mass-shifted reference marker maps a predictable distance from a corresponding native marker in a mass spectrometric display. 388. The method of embodiment 386, or any of the above embodiments, wherein the at least 1 mass-shifted reference marker is isotopically labeled. 389. The method of embodiment 386, or any of the above embodiments, wherein the at least 1 mass-shifted reference marker is labeled using at least one of H2, H3, heavy nitrogen, heavy carbon, heavy oxygen, S35, P33, P32, and isotopic selenium. 390. The method of embodiment 386, or any of the above embodiments, wherein the at least 1 mass-shifted reference marker is chemically modified. 391. The method of embodiment 386, or any of the above embodiments, wherein the at least 1 mass-shifted reference marker is at least one of oxidized, acetylated, methylated, and phosphorylated. 392. The method of embodiment 386, or any of the above embodiments, wherein the at least 1 mass-shifted reference marker is a nonhuman homolog of a human protein mass feature. 393. The method of embodiment 387, or any of the above embodiments, comprising digitally quantifying the corresponding at least one native marker in the mass spectrometric display. 394. The method of embodiment 387, or any of the above embodiments, comprising displaying at least 5 mass-shifted reference markers added to the sample. 395. The method of embodiment 387, or any of the above embodiments, comprising displaying at least 10 mass-shifted reference markers added to the sample. 396. The method of embodiment 387, or any of the above embodiments, comprising displaying at least 15 mass-shifted reference markers added to the sample. 397. The method of embodiment 387, or any of the above embodiments, comprising displaying at least 20 mass-shifted reference markers added to the sample. 398. The method of embodiment 387, or any of the above embodiments, comprising displaying at least 50 mass-shifted reference markers added to the sample. 399. The method of embodiment 387, or any of the above embodiments, comprising displaying at least 100 mass-shifted reference markers added to the sample. 400. The method of embodiment 387, or any of the above embodiments, wherein the at least 1 mass-shifted reference marker is present on a collection device prior to contacting the collection device to a sample. 401. The method of embodiment 387, or any of the above embodiments, wherein the at least 1 mass-shifted reference marker is added to the dried blood spot sample. 402. The method of embodiment 387, or any of the above embodiments, wherein the at least 1 mass-shifted reference marker is added to a resolubilized sample prior to mass spectrometric analysis. 403. The method of embodiment 386, or any of the above embodiments, wherein the mass features comprise at least one protein fragment. 404. The method of embodiment 386, or any of the above embodiments, wherein the mass features comprise at least one biomolecule. 405. The method of embodiment 386, or any of the above embodiments, wherein the mass features comprise at least one lipid. 406. The method of embodiment 386, or any of the above embodiments, wherein the mass features comprise at least one nucleic acid. 407. The method of embodiment 386, or any of the above embodiments, wherein the mass features comprise at least one hormone. 408. The method of embodiment 386, or any of the above embodiments, wherein the mass features comprise at least one drug. 409. The method of embodiment 386, or any of the above embodiments, comprising storing mass spectrometric analysis data on a computer. 410. The method of embodiment 409, or any of the above embodiments, comprising subjecting the mass spectrometric analysis data to a machine learning algorithm. 411. The method of embodiment 392, or any of the above embodiments, comprising correlating the at least one sample to an individual of known health condition status for a health condition, and performing a machine learning analysis on the at least one native marker. 412. The method of embodiment 411, or any of the above embodiments, wherein the at least one sample comprises at least 10 samples, and the correlating comprises correlating each sample to an individual source of that sample. 413. The method of embodiment 386, or any of the above embodiments, comprising displaying at least 25 mass features from the solubilized sample. 414. The method of embodiment 386, or any of the above embodiments, comprising displaying at least 30 mass features from the solubilized sample. 415. The method of embodiment 386, or any of the above embodiments, comprising displaying at least 40 mass features from the solubilized sample. 416. The method of embodiment 386, or any of the above embodiments, comprising displaying at least 50 mass features from the solubilized sample. 417. The method of embodiment 386, or any of the above embodiments, comprising displaying at least 100 mass features from the solubilized sample. 418. The method of embodiment 386, or any of the above embodiments, comprising displaying at least 200 mass features from the solubilized sample. 419. The method of embodiment 386, or any of the above embodiments, comprising displaying at least 500 mass features from the solubilized sample. 420. The method of embodiment 386, or any of the above embodiments, comprising displaying at least 1000 mass features from the solubilized sample. 421. The method of embodiment 386, or any of the above embodiments, comprising displaying at least 2000 mass features from the solubilized sample. 422. The method of embodiment 386, or any of the above embodiments, comprising displaying at least 5000 mass features from the solubilized sample.

Further understanding of the disclosure herein is gained in light of the Examples provided below and throughout the present disclosure. Examples are illustrative but are not necessarily limiting on all embodiments herein.

### EXAMPLES

### Example 1

### Blood Spot Biomarker Collection and Extraction

Whole blood samples are applied to a Noviplex DBS Plasma Card as indicated in FIG. 1. The whole blood is drawn through a separating layer comprising a separator to isolate plasma, and the plasma is directed to a plasma collection reservoir. The plasma contacts an isolation screen on a case card, and is dried for later analysis.

The spot is placed into an individual well for TFE/Trypsin (enzymatic) digestion for 24 hours. The digestion is quenched, transferred to an MTP plate and dried down. The sample is then reconstituted and subjected to mass spectrometric analysis.

### Example 2

### Alternate Blood Collection

Whole blood samples are applied to a Neoteryx Mitra blood collection device and subjected to processing as in Example 1. Blood is applied to a three dimensional absorbent structure rather than being spotted onto a two dimensional plane. As above, the sample is dried and does not need to be refrigerated.

### Example 3

### Repeatability of Mass Spectrometric Analysis

Blood spot samples were subjected to mass spectrometric analysis to assess the data diversity and repeatability of the measured samples.

A single set of dried plasma samples from a single plasma pool were spotted onto 16 dried plasma sample cards and subjected to 3 mass spec runs per card to generate 48 data sets. The results are shown in FIG. 2

Visual inspection of FIG. 2 indicates a remarkable degree of repeatability for mass spectrometric output among and across the 48 datasets.

The biomarker generation was assessed for multiple measures of repeatability. The results are shown in FIGS. 2-6 and in Table 1.

TABLE-US-00001 TABLE 1 #Tech. #Within-Card #DPS Reps/Detected CV's Between-Card Study Cards Card Features (median) CV (median) Technical 16 3 64,667 3.3-6.2% 9.0% Variability Repeated 12 4 65,795 5.1-6.3% 16.2% Sampling Variability Across 99 1 55,939˜25.6% Cohort Variability

Table 1 presents results of experiments to assess technical variability for a given sample, variability among repeated sampling of a common source, and variability across members of a cohort.

Among technical repeats of a given sample, 16 DPS cards were used, and three technical replicates were analyzed per card. 64,667 features were detected per replicate analyzed. Within card median coefficients of variation were calculated to range from 3.3% to 6.2%, while median between card coefficients of variation were determined to be 9.0%. These results are presented graphically in FIG. 3. These data correspond to the mass spectrometric results depicted as raw data in FIG. 2.

As an additional measure of the repeatability of biomarker generation, consecutively taken samples from a single collection incident were analyzed.

Among sampling repeats of a given sample, 12 DPS cards were used, and four technical replicates were analyzed per card. 65,795 features were detected per replicate analyzed. Within card median coefficients of variation were calculated to range from 5.1% to 6.3%, while median between card coefficients of variation were determined to be 16.2%. These results are presented graphically in FIG. 4.

These results indicate that the workflow used to measure biomarkers is highly repeatable.

Repeatability was also assessed across individuals within a cohort. Across individuals within a cohort, 99 DPS cards were used, and one spot was analyzed per card. 55,939 features were detected per replicate analyzed. Median between card coefficients of variation were determined to be 25.0%. These results are presented graphically in FIG. 5.

These results indicate that, even across separate cohorts having separate health status or health conditions, the majority of biomarkers measured in the assays did not substantially vary. Thus, one may conclude that the subset of biomarkers observed to vary across samples is likely to be enriched for biomarkers relevant to the health status or health condition varying between cohorts, and therefore informative as to health status or health conditions in additional cohorts or individuals.

### Example 4

Quantitative Capacity of Mass Spectrographic Results Obtained from Dried Plasma Samples

Mass spec results from dried plasma samples were obtained, and fragment signals corresponding to FDA-recognized marker proteins were assessed as to protein levels. As protein levels for these proteins in healthy individual plasma are well measured and published, these markers served as a control from which to assess the quantitative accuracy of the mass spectrometric data.

The results are presented in FIG. 6. Endogenous concentration is depicted across the x-axis, while normalized mass spec instrument response is seen on the y-axis. The dotted diagonal line approximates a perfect correlation between endogenous concentrations and normalized response. One sees that, across a range of at least 5 orders of magnitude, FDA proteins were detected at levels consistent with their FDA predicted levels. Measurements rarely differed by even an order of magnitude (see, for example, Transthyretin). The majority of proteins fell either along the dashed axis, or within or near to the grey shaded region representing only modest variation from the diagonal.

These results indicate that instrument response approximates endogenous plasma concentrations for samples extracted from dried plasma spots.

Similar verification of the quantitative capacity of approaches disclosed herein is presented in FIG. 7. FIG. 7 demonstrates that known and identified proteins have been identified via mass spectrometric analysis of dried blood spots using methods consistent with the disclosure herein. Proteins are ranked by protein concentration and ordered along the x-axis from greater to lesser concentration. The y-axis indicates normalized instrument response for the same proteins.

One observes that the instrument response correctly ordered the proteins as to their rank across 5-6 orders of magnitude. Abundant, common blood proteins are depicted at the upper left, while much rarer proteins such as transcription factors are found at the lower right.

These results further indicate that instrument response approximates endogenous plasma concentrations for samples extracted from dried plasma spots.

Quantitative capacity of the instrument response was further assessed by adding a known quantity of an exogenous protein to samples, and analyzing the protein levels indicated by the results of mass spectrometric analysis.

Gelsolin protein was spiked into plasma samples at known concentrations, and instrument responses were assessed.

The results are depicted in FIG. 8. The x-axis indicates deposited gelsolin protein levels. The y-axis indicates normalized instrument response. The dashed vertical line indicates the point at which deposited gelsolin is added at a level that is comparable to endogenous levels. The left and right panels depict results for two peptide fragments, indicated at the top of each panel, that map to the gelsolin protein.

As indicated in FIG. 8, normalized instrument response precisely and accurately reflects increases in gelsolin concentration resulting from addition of exogenous gelsolin.

### Example 5

### Mass Spectrometric Analysis Identifies Novel Protein Variants not Observable Through Genomic Analysis

Dried plasma samples were analyzed as disclosed herein, and the results were assessed so as to identify the identity of the resulting fragments. 10,306 unique spectra IDs were identified, corresponding to 9,900 unique feature IDs, mapping to from 2,242 to 2,290 proteins (with a 95% Confidence Interval). Within this peptide fragment dataset, 308 sequence variants were identified and 23 un-annotated ORFs were identified. 2,542 known biological post-translational modifications of proteins were identified and accurately measured, facilitating their use as biomarkers. Similarly, 406 novel post-translational modifications, not detected by previous mass spectral searches, were identified through this analysis, facilitating their use as biomarkers. Post-translational modifications are not largely accessible through nucleic acid-based sequencing. Thus, by demonstrating that these biomarkers are reliably detected, one can use these as biomarkers for health status or health condition assessment, but only if protein biomarkers are assessed, and only when the assessment demonstrates accuracy and repeatability consistent with the approaches of the disclosure herein.

### Example 6

Mass Spectrometric Analysis Accurately Classifies Individuals by their Health Status or Health Category

A feasibility study was conducted to demonstrate the utility of biomarker measurements obtained from mass spectrometric outputs for sample grouping and predictive classification. About 1,000 samples were collected by ProMedDx using an IRB-approved protocol. Samples were collected from 500 male vs. 500 female participants, 500 age under-50 vs. 500 age over-50; 500 Caucasian vs. 500 African-American. The data architecture indicates that there are approximately 125 samples in each unique 3 parameter classes. MS DPS proteomic data were analyzed to detect gender, age and race related signals that may be used to form informative panels for sample classification.

Results are shown in FIGS. 9-10. At FIG. 9 one sees the results of a classification predictive of sex of the sample origin. 32 age-matched male and female pairs were sorted using 16 MS features subjected to ten rounds of 10-fold cross validation using a PLSDA model. False positive rate is depicted across the x-axis, while true positive rate is depicted along the y-axis. The MS feature-based analysis correctly categorized samples into the sex of their source with an AUC of 0.96. In a control set where classes were randomized, the MS feature-based analysis categorized samples with an AUC of about 0.52, consistent with a random assignment into classes. For reference, an AUC of 1.0 represents a sorting that is 100% accurate, while an AUC of 0.5 is observed for random sorting into binary categories, as expected for example by a coin toss. Thus, as indicated by FIG. 9, MS feature-based analysis categorized samples with a remarkably high degree of accuracy, based in this case solely on analysis of MS-DPS derived fragment level data.

At FIG. 10 one sees the results of a classification predictive of race of the sample origin. 30 age-matched Caucasian and African American pairs were sorted using 28 MS features subjected to ten rounds of 10-fold cross validation using a Glmnet model. False positive rate is depicted across the x-axis, while true positive rate is depicted along the y-axis. The MS feature-based analysis correctly categorized samples into the sex of their source with an AUC of 0.98. In a control set where classes were randomized, the MS feature-based analysis categorized samples with an AUC of about 0.54, consistent with a random assignment into classes. Thus, as indicated by FIG. 10, MS feature-based analysis categorized samples with a remarkably high degree of accuracy, based in this case solely on analysis of MS-DPS derived fragment level data.

### Example 7

### MS-DPS Analysis Classifies Samples by Health Status

Samples from cohorts varying in colorectal cancer status were used to identify markers indicative of colorectal health. In a first set, 54 CRC and 54 control samples were analyzed using MS Features only. A PLS-DA model was adopted, relying upon 6 features.

The results are depicted in FIG. 11. The MS feature-based analysis correctly categorized samples into the CRC status of their source with an AUC of 0.76. In a control set where classes were randomized, the MS feature-based analysis categorized samples with an AUC of about 0.5, consistent with a random assignment into classes.

In a second set, 89 CRC and 207 control samples were analyzed in an analysis comprising MS Features and Age as biomarkers. The dataset was subjected to PLS-DA model, and 10 features were used to form a panel.

The results are depicted in FIG. 12. The MS feature-based analysis correctly categorized samples into the CRC status of their source with an AUC of 0.76. In a control set where classes were randomized, the MS feature-based analysis categorized samples with an AUC of about 0.49, consistent with a random assignment into classes.

In yet another analysis, an MS-DPS approach was used to develop a signal inactive of coronary artery disease (CAD). Samples were analyzed from individuals falling into one of two groups, having either 0 or severe (greater than 100) CAD risk score. 91 samples were scored using information regarding gender/age/site matched pairs.

The results are depicted in FIG. 13. The MS feature-based analysis correctly categorized samples into the CAD status of their source with an AUC of 0.71. In a control set where classes were randomized, the MS feature-based analysis categorized samples with an AUC of about 0.52, consistent with a random assignment into classes.

This example demonstrates that samples can be sorted according to characteristics indicative of patient health, such as CRC or CAD status in addition to patient identity, such as gender or race.

### Example 8

### Implementation of an Ongoing Patient Monitoring Regimen

Four patients were subjected to a 30 day monitoring regimen comprising daily acquisition of blood samples through dried blood spots. The samples were processed and the analyzed for trends indicative of health status. No health status changes were reported in any of the participants, and no patterns were observed in the participants' biomarker levels during the study.

This example illustrates that longitudinal monitoring of patient health through regular periodic sample acquisition is a viable heath assessment and monitoring approach. The samples were regularly provided by participants without ‘sample fatigue’ or other issues related to participation. Samples were repeatedly accurately measured, consistent with the disclosure including the prior examples herein. Importantly, patient health accurately correlated with MS DPS patient signal, in that no health events were predicted and no adverse health conditions were observed.

### Example 9

### High Throughput MS Protocol Modification Yields Rapid, High Quality Results

Mass spectrometric analyses were used to generate about 30,000 features per sample by employing a 30 minute liquid chromatography gradient. To increase throughput of the MS analysis pipeline, a portion of the gradient disproportionately responsible for a high density of meaningful data was identified, and an optimized gradient was developed so as to focus on this more information dense region of the gradient. By focusing on this region, the gradient time was reduced from 30 to 10 minutes, and the information content of biomarkers remained greater than 10,000.

The 30 minute and 10 minute gradients are depicted in FIG. 14. Data images resulting from 30 minute (left) and 10 minute (right) protocols are depicted in FIG. 15.

The modified protocol allows a rapid analysis of samples without sacrificing overall sample biomarker quality in some workflows consistent with the disclosure herein.

### Example 10

Biomarker Acquisition and Panel Construction from a Diversity of Data Sources

An ongoing health monitoring protocol is implemented for cohort of 40 patients. Biomarkers are monitored from a wide diversity of sources, as indicated in FIG. 16. Data collected includes physical data, personal data and molecular data, and includes glucose levels, blood pressure, cognitive well-being data, heart rate, and caloric intake, as well as molecular data such as mass spectrometric data obtained from plasma samples obtained as dried blood spots and obtained from captured exudates in breath samples. An example of raw mass spectrometric data generated from captured exudates in breath is given in FIG. 17. Biomarker and other marker data from multiple sources are integrated as part of a multi-source marker regimen, and depicted in FIG. 18.

Data is collected and analyzed in light of patient health reports throughout the health monitoring course. Markers, including in some cases biomarkers obtained from proteomic analysis of DBS samples and breath samples, and including in some cases other marker data such as weight, age and caloric intake, are used to profile participants throughout the course of the monitoring.

Markers are identified that correlate with patient health status changes during the course of the course. The markers are calculated to predict a health status or condition with an AUC of substantially above 0.65.

The markers are assembled into a panel for specific detection of the health status change using antibodies for specific protein biomarker detection, and using non-antibody approaches to obtain non-protein marker information. The resultant detection panel is assembled into a kit that is provided to the public as a specific test for the health condition or status.

### Example 11

Biomarker Acquisition Individual Health Monitoring from a Diversity of Data Sources

An ongoing health monitoring protocol is implemented for an individual.

Biomarkers are monitored from a wide diversity of sources, as indicated again in FIG. 16. Data collected includes physical data, personal data and molecular data, and includes glucose levels, blood pressure, cognitive well-being data, heart rate, and caloric intake, as well as molecular data such as mass spectrometric data obtained from plasma samples obtained as dried blood spots and obtained from captured exudates in breath samples. An example of raw mass spectrometric data generated from captured exudates in breath is given in FIG. 17. Biomarker and other marker data from multiple sources are integrated as part of a multi-source marker regimen, and depicted in FIG. 18.

Data is collected and analyzed over time. It is observed that markers implicated in glucose regulation and glucose levels are found to vary over the course of the protocol. Glucose levels are observed to be successively less regulated, but not at levels that would on their own indicate diabetes. Biomarkers correlating to glucose regulation, and implicated in diabetes, are found to change in levels monitored through the course of the monitoring. It is observed that mental acuity is affected in a manner that correlates with blood glucose levels. It is also observed that the magnitude of these changes scales roughly with an increase in patient weight.

Each of these markers shows some change, but none of these markers individually generates a signal strong enough to lead to a statistically significant signal indicative of progression toward diabetes. Nonetheless, the aggregate signal generated by a multifaceted analysis involving markers from a diversity of sources, including biomarkers from patient dried blood samples, strongly indicates a pattern trending toward the onset of diabetes.

Thus, the ongoing monitoring indicates that the patient is exhibiting early signs of diabetes, and that the severity of the response may scale with increase in patient weight.

A weight control regimen is initiated, and monitoring continues. It is observed that as measurements of caloric intake decrease, exercise increases and weight decreases, the overall marker signal indicating diabetes symptom progression decreases. However, a subset of the markers indicates that the risk of diabetes progression persists even with caloric reduction and exercise.

A medical professional in possession of a report detailing the results concludes the following. The patient is susceptible to diabetes. No diabetes-related damage has occurred because the monitoring regimen detected the health status well ahead of the demonstration of harmful symptoms. Progression of the disease can be checked through exercise, weight control and a regimented diet. However, the potential to develop diabetic symptoms remains.

### Example 12

### Targeted Detection of Proteins of Interest in Mass Spectrometric Results

Samples are obtained and processed as in Example 1. Prior to performing mass spectrometric analysis, the samples are supplemented with a set of labeled peptides or proteins corresponding to FDA-recognized marker proteins of acknowledged diagnostic relevance.

The supplemented samples are subjected to mass spectrometric analysis. At least 10,000 polypeptide fragments are observed, consistent with the results presented herein. At particular positions in the output, one observes labeled marker polypeptides that are distinguished from the native polypeptide signals. For at least some of these labeled marker polypeptides, one observes a native signal displaced from the marker signal by a regular, predicted distance corresponding to the difference between the labeled proteins and their native counterparts.

These signals are associated with native counterparts of the labeled proteins. By focusing on these marker adjacent native proteins, one readily determines levels of the FDA proteins of interest in the sample.

At the same time, in addition to the marker adjacent signals, the remaining at least 10,000 signals are also detected and are available for further analysis.

### Example 13

### Immunological Biomarker Panel Assessment

A panel of protein biomarkers informative of colorectal cancer was developed. The panel includes the proteins AACT, CATD, CEA, CO3, CO9, MIF, PSGL, and SEPR. The proteins are assayed is blood samples from individuals, and levels are assessed through an immunoassay kit involving antibodies to the panel constituents. The assay determines panel protein levels with a high degree of repeatability, such that colorectal cancer assessments are made with a high degree of sensitivity and a high degree of specificity.

### Example 14

### Marker Assisted Mass Spectrometric Biomarker Panel Assessment

The panel of example 13 is used for marker assisted mass spectrometric biomarker panel assessment. Samples are collected again from patient blood.

Heavy-isotope labeled AACT, CATD, CEA, CO3, CO9, MIF, PSGL, and SEPR proteins are added into the samples, and the samples are processed and subject to mas spectrometric analysis. Heavy isotope labeled polypeptide fragments are readily identified in the mass spectrometric results. For each identified heavy isotope labeled polypeptide in the results, a spot is detected exhibiting a displacement from the labeled polypeptide consistent with the spot being the unlabeled equivalent of the labeled polypeptide, present as a part of the sample rather than as a marker. Assisted by the heavy isotope markers, native AACT, CATD, CEA, CO3, CO9, MIF, PSGL, and SEPR levels are readily obtained in the sample.

Spot signal intensities are compared to marker signal intensities to facilitate the accurate determination of native spot signal, and ultimately of concentration of the corresponding protein or other biomarker in the original sample.

In addition, the mass spectrometric data includes information for over 30,000 additional protein and other biomarkers in the sample. The additional mass spectrometric data is available for independent corroboration of the colorectal cancer health analysis and for independent health assessments from the sample.

### Example 15

### Marker Assisted Mass Spectrometric Biomarker Panel Development

Samples are obtained from a number of individuals differing in a disease state. The samples are subject to mass spectrometric analysis, and biomarkers are identified that vary in signal in correlation with disease state. Biomarker identification is complicated by the high density of polypeptide spots on the output, requiring sophisticated data analysis to accurately call markers in mass spectrometric data.

Specific polypeptides that consistently co-vary with disease state are extracted and subjected to polypeptide sequencing, allowing identification of protein of origin for the markers.

Heavy isotope marker proteins are developed for each of the specific polypeptides that consistently co-vary with disease state.

Follow-on samples are obtained from a 10-fold greater number of individuals. Samples are supplemented with heavy labeled polypeptides of the identified biomarkers at known concentrations. The presence of the heavy labeled biomarker labels simplifies native biomarker identification in the mass spectrometric data output, such that more samples are analyzed in a substantially more accurate, high throughput analysis pipeline. Following sample spot identification, the biomarker labels reference spots are used to facilitate native sample spot quantification by comparing the reference spot signal strengths to the signal strengths of the native spots of interest.

The majority of the biomarkers are verified as being informative of the health status in the larger population. The verified biomarkers are selected as targets for a blood-based immunoassay to be provided as a kit for on-site sample collection and assessment.

### Example 16

### High Throughput Multi-Panel Assessment Via Marker Assisted Mass Spectrometric Analysis

Biomarker panels are developed to identify biomarker signatures in circulating blood for a number of disorders, including panels that individually are able to detect a risk of a broad range of early cancers and other asymptomatic pre-conditions. The panels, in combination, involve more than 200 protein biomarkers.

A first blood sample is taken from an individual. The sample is assayed to assess its biomarker panel profile. The panels are assessed using an immunoassay based approach. Measurements are accurately made, but the number of antibodies renders the sheer number of assays cumbersome to implement, resulting in a larger amount of sample being required, and more time spent in implementing the assays.

A second sample is taken from the individual. The sample is subjected to mass spectrometric analysis so as to assay biomarker levels in a single assay. A total polypeptide mass spectrometry profile is generated for the sample. Some biomarkers are identified and accurately quantified. However, the density of polypeptide signals on the total polypeptide mass spectrometry profile complicates the accurate identification and quantification of some of the markers, and some of the panels cannot be accurately assessed due to challenges in the data generation.

For each of the more than 200 protein biomarkers, a heavy isotope labeled marker protein is developed. Each marker protein is developed so as to migrate upon mass spectrometric analysis at a predictable offset from its unlabeled native counterpart in a given sample, and to be readily detected in mass spectrometric output.

A third sample is taken from the individual. Heavy isotope labeled marker proteins are added to the sample at known concentrations for each of the more than 200 protein biomarkers. The sample is subjected to mass spectrometric analysis and the output is analyzed.

Using the marker protein mass spectrometric fragments as guides, mass spectrometric signals corresponding to native biomarkers in the samples are readily identified. Following sample spot identification, the biomarker labels reference spots are used to facilitate native sample spot quantification by comparing the reference spot signal strengths to the signal strengths of the native spots of interest.

As compared to analysis of the first sample and the second sample above, it is observed that the third sample is analyzed more accurately, more quickly, and with substantially less reagent use or benchtop manipulation that the first sample. It is also observed that the third sample is analyzed in a comparable amount of lab bench time to the second sample, but the downstream analysis related to calling of mass spectrometric signals as corresponding to one or another biomarker is substantially faster, easier and more accurate in the biomarker-labeled third sample, and native spot quantification is considerably more accurate.

### Example 17

### Large Scale High Throughput Multi-Panel Assessment Via Marker Assisted Mass Spectrometric Analysis

Over 1,000 labeled biomarker reference standards as described above are introduced into a blood sample at known concentrations prior to subjecting proteins in the sample to mass spectrometric analysis. The biomarker reference standards are heavy isotope labeled so as to migrate in mass spectrometric analysis at a predicted offset from the native protein, and to be easily detected independent of their mass spectrometric labeling, and with a high degree of confidence.

The over 1,000 labeled biomarkers are readily detected in the mass spectrometric analysis. For each labeled biomarker, it is readily identified where the native, unlabeled biomarker corresponding to the labeled biomarker is expected to migrate.

For some biomarkers, a mass spectrometric signal is detected for a distinct spot at the predicted offset from the labeled biomarker. The signal is quantified by comparison to reference spot signal intensity on mass spectrometric visualization, and assigned to be representative of the native biomarker level.

For some biomarkers, a mass spectrometric signal is detected at the predicted offset from the labeled biomarker, but the signal is part of a spot that is not distinctly separated from adjacent spots on the mass spectrometric output. Because the adjacent labeled standard is available as a reference, one can accurately identify where the native biomarker is expected to be in light of the predicted offset between the labeled and unlabeled polypeptides. One can also readily determine the expected size of the spot corresponding to the native biomarker by comparison to the size of the spot of the labeled standard. The portion of the spot expected to correspond to the native protein is quantified and assigned to be representative of the native biomarker level.

For some biomarkers, a mass spectrometric signal is detected corresponding to the labeled biomarker, but no signal is detected at the predicted offset from the labeled biomarker. It is concluded that the native biomarker is not present in the sample subjected to the mass spectrometric analysis.

For some biomarkers, a mass spectrometric signal is detected corresponding to the labeled biomarker. No spot is detected at the predicted offset from the labeled biomarker, but multiple spots are detected very close to the predicted offset location. In the absence of the labeled biomarker standard, one could readily assign any of these spots to be representative of the native biomarker. However, using the labeled biomarker as a reference, one observes that none of the local spots correspond to the offset position predicted for the native biomarker. In the absence of the labeled biomarker, it would be difficult to call any of the spots as either being or not being a spot corresponding to the biomarker of interest. In light of the added accuracy gained by using the labeled biomarker offset, it is concluded that the native biomarker is not present in the sample subjected to the mass spectrometric analysis.

It is observed that the over 1,000 native biomarkers are substantially more accurately assayed when the sample analysis comprises labeled marker polypeptides as guides.

### Example 18

Large Scale High Throughput Multi-Panel Assessment Via Mass Spectrometric Analysis for Database Generation is Complicated by Challenges in Data Acquisition

Over 1,000 biomarkers are identified as relevant for generation of a biomarker database. Blood samples collected from dried blood spots from over 1,000 individuals each having a known disease state for a number of independent conditions. Sample collection is repeated monthly over the course of five years.

Samples are subjected to mass spectrometric analysis to quantify biomarker levels in each sample. It is found that biomarkers are identified and quantified at a level of no greater than 90% confidence. Challenging factors include biomarker spot signals that run into one another, or are otherwise present in dense regions of a mass spectrometric output, and the lack of reference signals of known concentration to use as standards. As a result, accurate quantification and condiment calling of signal absence is difficult. Analysis is facilitated by manual inspection, but the absence of an automated data acquisition pipeline complicates the workflow, and hampers both throughput and overall database accuracy.

### Example 19

### Large Scale High Throughput Multi-Panel Assessment Via Marker Assisted Mass Spectrometric Analysis for Database Generation

Over 1,000 biomarkers are identified as relevant for generation of a biomarker database. Blood samples collected from dried blood spots from over 1,000 individuals each having a known disease state for a number of independent conditions. Sample collection is repeated monthly over the course of five years.

Prior to mass spectrometric analysis, heavy labeled marker proteins for each of the over 1,000 biomarkers are added at known concentrations, such that the heavy labeled proteins will yield polypeptides that migrate at a predictable offset from their native unlabeled counterparts, and such that the labeled polypeptides are readily identified in the sample.

Samples are subjected to mass spectrometric analysis to quantify biomarker levels in each sample. It is found that biomarkers are identified and quantified at a level of greater than 99% confidence. Native polypeptide spots are readily identified by their predicted offset from corresponding labeled marker standards, such that ‘fused’ spots are readily resolved and such that spot predicted locations are readily identified in spot-dense regions, facilitating more accurate absence calls as well as presence calls and measurements. By comparing the native spots to the reference spots of known original concentration, one can readily quantify the native spots to a high degree of accuracy.

The measurement process is readily automated without the requirement for manual assessment, greatly facilitating high-throughput data generation. The accuracy of the offset calculations in data acquisition further improves database overall accuracy.

### Example 20

### Combining Label-Free Proteomics and MRM Techniques in a Single Method

A pooled plasma sample was used as the matrix for evaluating Stable Isotope Standard (SIS) peptide response in both a standard plasma workflow and spotted onto DPS cards. All samples were digested in triplicate with a TFE based trypsin digestion protocol. Each sample was lyophilized after digestion and reconstituted with a panel of 641 SIS peptides comprising 392 proteins associated with colorectal cancer. The peptides were selected by several performance characteristics (i.e. peak abundance, CV's, precision, etc.) during the development of a MRM assay for the biomarker detection of patients with elevated CRC risk. Each sample was analyzed on an Agilent 6550 qTOF instrument with an optimized 32 minute gradient utilizing both MS1 and MS2 spectral acquisition modes.

The 641 SIS peptides (encompassing 392 proteins) used here were originally selected as part of a colorectal cancer panel, though the individual proteins are also associated with other indications (e.g. oncology, inflammation). These peptides were used to demonstrate the capabilities of the HRMS/SIS approach across a range of proteins on two sample formats (plasma, DBS/DPS).

A total of 24-10 uL injections comprising a dilution series of both neat plasma and DPS plasma digests were individually processed on an Agilent 6550 qTOF. From both the neat plasma and the DPS plasma experiments, the molecular features from the HRMS data were extracted and associated across the injections. From this data, the quantitative response of the SIS peptides across the dilution series was evaluated. Approximately 500 of the 641 SIS peptides showed a quantitative change with dilution level. The dynamic performance for each peptide was evaluated in terms of linearity, reproducibility and lower limit of quantitation. For the non-labeled features in the samples, the number of features was used to estimate the total information content in the data. MS2 data acquisition of the selected molecular features was used as further confirmation. Approximately 30,000 molecular features (z=2-4) were found in the samples on average, for both the neat and DPS plasma experiments, highlighting the richness of the data that is accessible through HRMS instrumentation. Further analysis quantifying molecular feature reproducibility, dynamic range, and a comparison between the neat and DPS experiments will also be presented.

Additional 10-10 uL injections of DPS plasma digest reconstituted with the SIS peptide panel at 158 fmol/uL were processed by LCMS. The data was extracted as described above. Median CV's of 15.3% for molecular features and 5.1% for the detected SIS peptides were observed.

Mass spectrometric output for the sample is presented in FIG. 19A. The image depicts both the benefits and the challenges of mass spectrometric analysis. Greater than 10,000 spots are detected.

At FIG. 19B, one sees the same output, but overlaid with the positions of exogenously added heavy labeled markers. The presence of the markers allows one to identify related spots in the mass spectrometric output corresponding to native proteins of particular interest.

This example, illustrated in FIGS. 19A and 19B, demonstrates the ability to quantify 100-1000's of known proteins, with simultaneous measurement of >30,000 molecular features.

### Example 21

### Quantification of SIS Marker Signals in Mass Spectrometric Sample Data

The 641 SIS peptides (641 polypeptides encompassing 392 proteins and 1552 transitions) of Example 20 were introduced at varying concentrations into aliquots of plasma and dried plasma extracted biomarker samples, and subjected to mass spectrometric analysis.

SIS markers were introduced to sample aliquots at 8 concentration levels ranging up to 500 fmol/uL. Each run is measured in triplicate. Each experiment (plasma and dried plasma spot) is run on QTOF and QQQ with the same gradient to facilitate cross-collection method comparisons. QTOF data were subjected to further analysis presented below. Marker spots were subjected to automated identification and putative marker spot signals were quantified. The results for a representative list of markers are presented in FIG. 20. For each polypeptide graph, marker concentration is depicted on the x-axis and spot signal intensity (as area on the instrument response output) is depicted on the y-axis. Spot calls that are likely accurate are depicted as filled circles having black outlines. Putative native sample spots miscalled as marker spots are depicted as light grey spots lacking outlines.

One sees that, for all polypeptide markers depicted in FIG. 20 (and representative of the larger number of polypeptide markers analyzed overall) a clear, strong linear correlation is observed between concentration (fmol/uL, ranging from 0 to 500, as indicated on the x-axis of the bottom-most file of panels) and spot signal strength. These results indicate that marker polypeptides are readily identified, and that their spot signal strength varies linearly with concentration, confirming both the efficacy of the identification process and their utility as markers to assist in quantification of native spots of comparable signal strength.

Occasional spot miscalls, such as seen in peptide 6, second panel of the second row, are informative for a number of reasons. Fist, even pronounced miscalls, as with peptide 6, do not disrupt the overall linear relationship between marker concentration and spot signal. Secondly, pronounced and even modest miscalls (peptides 6 and 3, for example) are readily identified by the impact that they have on the overall correlation between concentration and spot signal response. Thus, correlations between concentration and spot intensity serve as a quality-control check for spot calls. By flagging markers for which a spot miscall may have occurred, they provide a further tool for increasing the overall accuracy of final mass spectrometric results.

Viewing the results in aggregate, one sees that for both the standard plasma and the dried plasma spot samples, 641 SIS marker polypeptides were used. For the standard plasma samples, 634 (99%) of these markers showed observable peaks at least once; 627 (98%) exhibited at least 2 observed peaks; 622 (97%) exhibited at least 3 observable peaks; 605 (94%) exhibited at least 3 consecutive peaks in the range of 50-500 fmol/uL concentration, of which 513 (80%) showed an r-squared value of greater than 0.8, and 490 (76%) showed an r-squared value of at least 0.9).

Comparable numbers for the dried plasma samples are as follows. 625 (98%) of these markers showed observable peaks at least once; 613 (96%) exhibited at least 2 observed peaks; 597 (93%) exhibited at least 3 observable peaks; 579 (90%) exhibited at least 3 consecutive peaks in the range of 50-500 fmol/uL concentration, of which 515 (80%) showed an r-squared value of greater than 0.8, and 498 (78%) showed an r-squared value of at least 0.9).

These results indicate that marker polypeptides are accurately, repeatably identified and quantified in naive sample mass spectrometric outputs. These results are consistent with the use of SIS peptides in quantification of native equivalents of marker polypeptides, and in the quantification of samples overall.

### Example 22

### SIS Marker Development and Details

The polypeptide markers discussed above were assembled as follows. This approach is broadly relevant for development for markers for a broad range of disorders, conditions or other categorizations.

A search of published data (literature and public databases) was performed, from which 431 CRC-related proteins were selected for developing an MRM assay. Following the optimization of liquid chromatography (LC) and mass spectrometry (MS) conditions, the specificity, linearity, precision and dynamic range of the assay was assessed for 8806 transitions from 1006 proteotypic peptides representing the 431 proteins. A review of the feasibility data resulted in further optimization with the final method measuring 1552 top performing transitions (with a minimum 2 transitions per peptide) specific for 641 peptides, representative of 392 of the originally selected 431 CRC-proteins. This final MRM method was subsequently used to evaluate 1045 individual patient plasma samples that were pre-analytically processed by immunodepletion and tryptic digestion.

Using a single multiplexed MRM assay, we evaluated the 392 candidate CRC protein markers in a study with 1045 patient samples. LC gradient optimization was performed on an Agilent 1290 UHPLC-6550 QTOF system using reversed phase separation performed on a C18 column. Collision energy (CE) optimization was performed on two Agilent 1290 UHLPC-6490 QQQ instruments. 6 CE steps were tested for each of 8806 transitions. The optimal CE was selected based on peak AUC abundance and the lowest CV of 3 technical replicates. Analytical performance based on specificity, linearity, precision and dynamic range was assessed for all 8806 transitions using a half-log serial dilution of a stable isotope standards (SIS) peptide mixture. Plasma spiked with the same SIS mixture was used to evaluate matrix interference and confirm transition specificity. Three technical replicates were collected for each experiment condition to assess the assay precision. The transitions for each peptide were automatically ranked based on analytical performance and the top two transitions per peptide were selected for each protein. Following data review, the final MRM method was comprised of 1552 transitions from 641 peptides representing 392 proteins. Transition concurrency was capped at 90 transitions for each 42-second LCMS acquisition window across the 32-minute LC gradient. The final MRM assay was used to quantitate 392 CRC-proteins in 1045 individual patient blood plasma samples. The data generated from this study was used in classifier analysis. Identification of a plasma-based CRC peptide signature is useful to identify individuals of elevated CRC risk, thereby encouraging these patients to undergo recommended colonoscopies.

This example demonstrates how SIS marker polypeptide sets are developed and, consistent with the results above, indicates how they are used for automated, accurate quantification of native biomarkers in patient samples analyzed through mass spectroscopy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of biomarker database generation comprising identifying a biomarker set to include in a database; obtaining reference biomarker molecules that comprise biomarker components that differ in mass spectrometric migration from the protein biomarkers; obtaining at least one sample to assay for inclusion into the database; providing the reference protein biomarker molecules to the sample; subjecting the sample to mass spectrometric analysis to generate a mass spectrometric analysis output; identifying the reference biomarker molecules in the mass spectrometric analysis output; and scoring mass spectrometric spots predictably offset from the reference protein biomarker molecules as spots indicative of reference protein biomarker molecules.

2. The method of claim 1, wherein the reference biomarker molecules are chemically modified.

3. The method of claim 1, wherein the reference biomarker molecules are at least one of oxidized, acetylated, methylated, and phosphorylated.

4. The method of claim 1, wherein the at least one sample comprises a dried plasma sample.

5. The method of claim 1, wherein the at least one sample comprises a collected breath sample.

6. A processor comprising a memory unit configured to store data indicative of health status categorization in a comparison sample, said memory unit comprising: storage capacity configured to receive reference mass spectrometry data for at least 20 mass spectrometric values derived from each of a plurality of analyzed dried samples; storage capacity to correlate said at least 20 mass spectrometric signals derived from each of a plurality of analyzed dried blood samples to non-mass spectrometry data comprising at least one of age of sample source individual, time of collection, geographical region of collection, demographic information, blood glucose levels at time of collection, sleep history at time of collection, and mental alertness at time of collection; a comparison unit configured to receive at least one individual dataset comprising mass spectrometry data for at least 50 mass spectrometric signals derived from each of a plurality of analyzed dried blood samples and non-mass spectrometry data comprising at least one of age of sample source individual, time of collection, geographical region of collection, demographic information, blood glucose levels at time of collection, sleep history at time of collection, and mental alertness at time of collection; and compare said individual dataset to said reference mass spectrometry data, such that an assessment as to whether the individual data set differs significantly from the reference dataset is made.

7. A method of biomarker data accumulation comprising obtaining a dried fluid sample from at least one subject, volatilizing the dried fluid sample, subjecting the sample to mass spectrometric analysis, and identifying at least 20 biomarkers in the mass spectrometric analysis.
